(19) United States
(12) Patent Application Publication
(10) Pub. No.: US 2024/0379193 A1
(43) Pub. Date: Nov. 14, 2024

(54) Process for Practicing Fractal Science using KFE

(71) Applicant: Gregory Marcus Friedlander, Mobile, AL (US)

(72) Inventor: Gregory Marcus Friedlander, Mobile, AL (US)

(21) Appl. No.: 18/196,930

(22) Filed: May 12, 2023

Related U.S. Application Data
(63) Continuation-in-part of application No. PCT/US22/20517, filed on Mar. 16, 2022.
(60) Provisional application No. 63/451,300, filed on Mar. 10, 2023.
(60) Provisional application No. 63/441,856, filed on Jan. 30, 2023.
(60) Provisional application No. 63/426,647, filed on Nov. 18, 2022.
(60) Provisional application No. 63/422,041, filed on Nov. 3, 2022.
(60) Provisional application No. 63/402,977, filed on Sep. 1, 2022.
(60) Provisional application No. 63/399,286, filed on Aug. 19, 2022.
(60) Provisional application No. 63/343,161, filed on May 18, 2022.

(51) Int. Cl. G16C 60/00; G16C 10/00

(52) U.S. Cl. CPC G16C 60/00; G16C 10/00

(57) ABSTRACT

The present invention relates to a method for improving processes in various undertakings by employing Key Fractal Elements (KFE). The method involves utilizing KFE to affect change in CT states and interpret matrices of CT states. By applying KFE elements, categorization, prediction, manipulation, and the design of radiation matrices for electronics, solar, thermal, fusion, and radioactive energy applications are achieved. The invention further encompasses the use of KFE to modify frequency-based systems, enhancing energy generation, transmission, utilization, and storage efficiency. Additionally, base transitions govern matrix composition and interaction, while KFE enables compression, decompression, and dimensional variations in CT states and matrices. The design of molecules, including atomic and molecular matrices, is optimized based on KFE principles. The controlled absorption and spew of CT state exchanges within matrices, as well as the targeting of AuT plasma, further enhance the effectiveness of the invention. By incorporating KFE, improved balance, plasma fulcrums, and efficient CT state interactions are achieved, leading to advancements in various fields. The application of KFE in reaction processes, fuel utilization, and energy management contributes to superior matrix changes and efficient pretime informational exchange. Overall, the present invention provides a comprehensive approach to enhancing processes in diverse domains through the utilization of Key Fractal Elements.

### INTRODUCTION

The concept of fractals has fascinated scientists, mathematicians, and researchers for decades. Their intricate patterns and self-similarity across scales have captivated our imagination. In recent years, a breakthrough has occurred with the discovery of key fractal elements (KFE) and their remarkable potential to revolutionize various industries.

This application covers the use of KFE in fusion, fission, chemistry, polymer chemistry, hydrogen extraction, electronics, batteries, quantum computing, and AI. By incorporating KFE, these fields can experience unprecedented advancements, unlocking new possibilities and shaping the future. KFE is defined by the mathematical framework and its refinement, providing a basis for further development. Application of KFE involves utilizing it to bring about changes in CT states, interpreting and categorizing matrices of CT states.

The process entails employing KFE elements for categorization, prediction, manipulation, designing radiation matrices, and enhancing frequency-based systems.

Base transitions governing matrix composition and interaction, including spatial, energy, atomic, chemical, electrical, biological, and large structures, are considered key fractal elements.

KFE enables the compression or decompression of CT states and the formation of matrices of CT states. KFE incorporates concepts such as CT states, fpix equations, fuse length, MI, defined spirals, overlapping and exponential compression features, pretime change characteristics, spiral alignment, CT state sharing, collisions as information exchange, net compression/decompression, balance and imbalance, changing base numbering, and net pretime change characteristics.

KFE finds application in field modeling, mechanical interactions, and energy conversion systems.

KFE facilitates changes in KFE and the use of KFE energy converters for staged fuels, utilizing fpix, MI, and exponential changes for specific fractal results.

KFE influences the design of matrices, molecular structures, dimensional variations, absorption and spew control, and proximity of states.

KFE allows for control over where absorption and spew occur within CT state matrices, influencing the design of machines and reactions.

Reacting chemicals using KFE enhances matrix changes, maximizes the release of pretime informational change, and optimizes the use of energy states.

AuT methodology enables the focus on leveraging KFE to maximize the amount of pretime change, control pressure, energy, and reactants generated.

KFE encompasses AuT plasma, stepped transitions, categorization, chemistry, biology, and other relevant features.

Balance is a key aspect of KFE, observed in atomic and molecular structure about the lower ct state or plasma fulcrum, folding patterns, magnetic repulsion effects, and high- and low-pressure system interactions.

Examples include:

### Fusion:

By applying KFE to fusion processes, we can harness the inherent power of atomic fusion more efficiently. The precise control of energy release, dimensional variations in matrices, and targeted absorption and spew of CT state exchanges enable us to achieve optimal fusion reactions. This technology promises to provide a clean, sustainable, and abundant energy source for the world.

### Fission:

KFE offers a transformative approach to fission reactions, allowing for enhanced safety, efficiency, and waste management. By utilizing KFE's ability to manipulate dimensional variations, control the release of pretime informational change, and design optimal matrices, we can maximize the energy output while minimizing the risks associated with fission processes.

### Chemistry:

In the realm of chemistry, the application of KFE opens up a realm of possibilities. The ability to compress or decompress CT states, form matrices, and precisely control base transitions within chemical reactions brings unprecedented control and predictability. This paves the way for the development of novel compounds, efficient reaction pathways, and tailored chemical processes.

### Polymer Chemistry:

KFE's influence extends to the realm of polymer chemistry, where dimensional variations play a crucial role. By utilizing KFE's mechanisms, we can introduce multiple dimensional frameworks into polymer layups, enhancing their strength, stability, and functionality. The precise manipulation of matrices and the utilization of fractally relevant shapes lead to the creation of advanced polymers with superior properties.

### Hydrogen Extraction:

The efficient extraction of hydrogen, a vital component of clean energy solutions, can be revolutionized through the application of KFE. By leveraging KFE's ability to control pressure, reactant matrices, and energy release, we can optimize the process of hydrogen extraction, making it more sustainable, cost-effective, and scalable.

### Electronics:

KFE holds immense potential for transforming the field of electronics. By employing KFE's principles, such as dimensional variations, balance and imbalance, and the design of fractally relevant structures, we can enhance the performance, efficiency, and reliability of electronic systems. This leads to the development of advanced devices, improved circuitry, and next-generation technologies.

### Batteries:

The energy storage sector can benefit greatly from the integration of KFE. By utilizing KFE's capacity to modify frequency-based systems, increase energy generation, and optimize storage and transmission, we can overcome the limitations of current battery technologies. This opens avenues for high-capacity, fast-charging, and long-lasting energy storage solutions.

### Quantum Computing:

KFE's unique attributes align seamlessly with the complex realm of quantum computing. By leveraging KFE's ability to manipulate CT states, fractal alignment, and dimensional variations, we can enhance the accuracy, stability, and computational power of quantum systems. This paves the way for groundbreaking advancements in data processing, cryptography, and simulations.

### AI:

Artificial intelligence systems can benefit significantly from the incorporation of KFE. By utilizing KFE's principles, including dimensional variations, balance and imbalance, and pretime change characteristics, we can enhance the efficiency, adaptability, and decision-making capabilities of artificial intelligence systems. KFE enables the design of AI algorithms that can better interpret and categorize complex data matrices, leading to more accurate predictions, improved machine learning models, and advanced problem-solving capabilities. The incorporation of KFE empowers AI to achieve unprecedented levels of performance and sophistication across various domains.

### Overall Integration:

By integrating KFE across these diverse fields, we unlock synergistic effects and pave the way for interdisciplinary breakthroughs. The ability to utilize KFE in a unified manner enhances the overall efficiency, accuracy, and reliability of these applications. Moreover, the transferability of KFE principles allows for seamless collaboration and knowledge sharing among different industries, fostering innovation and driving societal progress.

### IN THE DRAWINGS

FIG. 1 shows a representative view of one form of ct1 solution folding.

FIG. 2 shows a representative view of ct3 to ct4 compression.

FIG. 3 shows a representative view of magnetism from electricity based on fractal modeling and associated magnetic attraction.

FIG. 4 shows a representative view of magnetic repulsion based on fractal modeling.

FIG. 5 shows a representative view of the growing proton count for protons based on fractal modeling.

FIG. 6 shows a representative view of carbon based on fractal modeling.

FIG. 7 shows a representative view of a larger atom neutron count based on fractal modeling.

FIG. 8 shows a representative view of force transitions using fractal modeling.

FIG. 9 shows a method of using force transitions using fractal modeling.

FIG. 10 shows a second method of using force transitions.

FIG. 11 shows an alternative view of the method in FIG. 10.

FIG. 12 shows a representative view of hydrogen separation using fractal modeling.

FIG. 13 shows a representative view of water using fractal modeling.

FIG. 14 shows fusion modeling using fractal modeling.

FIG. 15 shows a side view of the model shown in FIG. 15.

FIG. 16 shows a representative view of a virtual chamber used in FIG. 15.

### SPECIFICATION

In the realm of physics, the concept of time has always been a fundamental and intriguing aspect of our existence. However, beneath its seemingly linear and constant nature lies a deeper understanding of the dimensional changes that underpin its very existence. This essay delves into the concept of pretime, an intricate framework of dimensional transformations that serve as the building blocks for the emergence of time as a form of “stop frame animation.” Through a technical exploration of pretime, we aim to shed light on the profound interplay between dimensions and time.

### Understanding Pretime:

Pretime can is the precursor to time, a realm where dimensional changes occur and set the stage for the unfolding of temporal events. At its core, pretime represents a dynamic state where the fabric of reality undergoes transformations, creating a sequential progression akin to stop motion animation. This concept challenges the conventional notion of time as a linear flow, revealing a deeper connection between dimensions and the perception of time.

### Dimensional Changes and Time Formation:

Within the framework of pretime, dimensional changes serve as the fundamental catalysts for the emergence of time. These changes occur on a subatomic level, shaping the behavior of particles and influencing the flow of events. Through intricate mathematical models and rigorous experimentation, scientists have begun to uncover the intricate relationship between dimensional transformations and the perception of time.

One significant aspect of pretime is the compression and decompression of dimensional states. These transitions, governed by intricate fractal mathematics, give rise to the sequential nature of time. The compression of dimensional states results in the animation-like freezing of events, while their subsequent decompression facilitates the perception of time's flow. This fractal compression and decompression, influenced by factors such as energy, spatial interactions, and atomic structures, form the basis for the stop frame animation-like characteristic of time.

### The Role of Key Fractal Elements (KFE) in Pretime:

Key fractal elements (KFE) play a crucial role in the formation and manipulation of pretime. KFE encompasses a range of mathematical principles, structural patterns, and dimensional features that govern the behavior of particles, systems, and phenomena. These elements include concepts such as fractal spirals, balance and imbalance, dimensional variation, energy exchange, and the intricate interplay between different CT states.

By harnessing KFE, scientists can design and manipulate pretime transitions, exerting control over the compression, decompression, and manipulation of dimensional states. The incorporation of KFE in pretime studies enables a more comprehensive understanding of the underlying principles governing time formation, opening avenues for innovative applications across various scientific disciplines.

### Applications and Implications:

The exploration of pretime and its relationship with dimensions and time has far-reaching implications for multiple scientific domains. By unraveling the intricacies of pretime, researchers can gain new insights into fundamental physical phenomena, refine models for energy generation, optimize chemical reactions, improve computational algorithms, and enhance our understanding of complex systems. In the realm of energy, the manipulation of pretime transitions through the application of KFE holds great promise. It offers the potential for more efficient fusion and fission processes, enabling the development of sustainable and clean energy sources. Moreover, the incorporation of pretime principles in electronics, batteries, quantum computing, and AI can revolutionize these fields, leading to breakthroughs in information processing, energy storage, and intelligent decision-making systems.

Mathematical proof of Utility:

A fractal algorithm-based universe must have base equations which are ubiquitous. For clarity, we begin with fpix, the equation giving rise to the denominator of pi, the ubiquitous phenomenon incorporating the equation is curvature. The details are important, but the nature of Key Fractal Elements (KFE) which give us control over features of the universe using the new modeling, including the precise transitions will be developed over time. The process of using KFE to interpret and control interactions with a universe driven by pretime change can be identified without all of the refinements which will come about. Pretime refers to the dimensional changes which occur which give rise to time as stop frame animation.

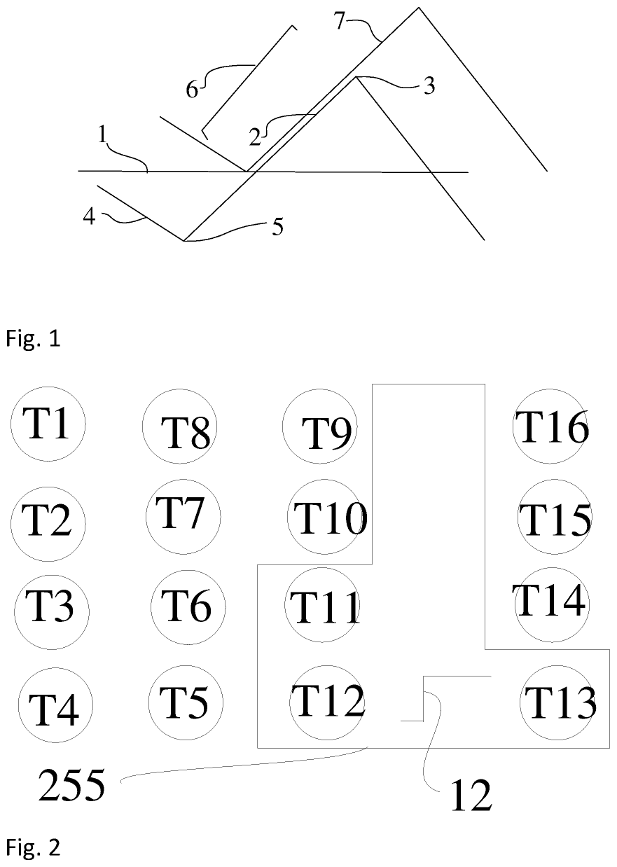

At the levels where we live, there are at least three choices, Fibonacci (1, 2, 3, 5, 8, 13), fpix (1, 3, 5, 7, 9) and the building sum (1, 2, 5, 8, 3, 2, 7, 12) which results from the absolute value of “fused” values of fpix over time as shown in FIG. 1. An analysis of observations shows that all of these solutions to f(x) in the iterated equation CTx=f(x)\hat{ }2\hat{ }x evolve and devolve as is the case for this unique fractal, fpix which must itself evolve from and according to the more base fractal leading to the quantum count.

fₚᵢ(x)=−1^{x}+2x*−1^{x-1}

Pi(n)=Pi(n−1)+[(x/′fpix(n)] where Pi(n−1) for n=1 is the numerator for pi at the level of compression under consideration. So for Pi4, the pi we are used to dealing with, Pi(1)=+4+4/fpix(n) for the first solution and Pi(1)+4/fpix(n) for the second solution. By focusing on a quantum state, fpix; AuT is able to look beyond fractals to the bits which give rise to the universe for the first time.

The modes of compression are not mutually exclusive. Fpix works up to the neutron, and at the neutron there is a visible shift to MI; but both exist together at the atomic level and the analysis and reasons for the transitions need to be refined. Unlike other patents, this series goes back to the moment of dimensional creation, even before the moment of creation, but patents are not required to do so to show utility and features inherent in the basic modeling will be refined.

| Modeling of force/compression | | | | | | | | | |
|---|---|---|---|---|---|---|---|---|---|
| (2f(n)\hat{ }(2\hat{ }n) using MI | | | | | | | | | |
| | | | compression | | | | | | |
| | | | ratio | Modeling using fpix instead of f-series | | | | | |
| N | 2*f(n), f(n) = MI | 2\hat{ }n | 2f(n)\hat{ }2n | n | f(n) | 2\hat{ }n | compressi [symbol] | ct1 states | Observed |
| 1 | 2 | 2 | 4 | 1 | 0 | 2 | 0 | 0 | space 1 |
| 2 | 4 | 4 | 256 | 2 | 1 | 4 | 16 | 16 | space 2 |
| 3 | 6 | 8 | 1.68E+06 | 3 | −3 | 8 | 1679616 | 26873856 | space 3 |
| 4 | 10 | 16 | 1E+16 | 4 | 5 | 16 | 1E+16 | 2.69E+23 | neutron |
| 5 | 16 | 32 | 3.40282E+38 | 5 | −7 | 32 | 4.74E+36 | 1.27E+60 | black hole [symbol] |
| 6 | 26 | 64 | 3.61655E+90 | 6 | 9 | 64 | 2.17E+80 | 2.8E+140 | blackhole [symbol] |

[symbol]

indicates data missing or illegible when filed

On the left, above, is modeling based on F-series, the traditional, but not necessarily favored method, which uses fpix and uses zero for the solution for fpix(n) where n=1, so that you get 2\hat{ }5 for fpix(n)=5 for n=4. In such a case the relative strength of gravity on the right is 2f(n)\hat{ }(2\hat{ }n) for n=2 vs n=5 for the strong force.

FIG. 1 shows not only “folding” but also 2\hat{ }n changes and a positive and negative series. If these values are summed over time based on constant sequential generation the result can be graphed showing folding and exponential compression which is inherent since it reflects adding 2 repeatedly. FIG. 1 reflects the natural folding of fpix solutions but it adds a second line 7, offset slightly for clarity, to refleCT the possible origin of overlap and shared information reflecting the baseline 1 (zero), initial positive results 2, initial negative results 4, initial negative inflection point 5, initial positive inflection points 3 and the shared area 6. So the parts can be seen they are offset, but this is merely representative of one way of approaching a more complex problem. The entire universe is frozen between quantum states and the movement of the system occurs as the quantum count changes, changing fuse lengths for all quantum bits which, when the fuse length of the bit reaches 0 changes the bit from positive to negative and increases the fuse length by L(n)+2 (the prior fuse length plus 2) and begins timing the change rate again.

FIG. 1 is a plot of “fused” solutions to fpix, where the fuse changes according to a quantum count (n=n+1) and the solutions change from their current solution fpix(x) to the next fpix(x+1) after the quantum count equals the fuse length. (e.g. when n=3 for fpix=−3) after which the count begins again for the fuse length which is now, in the example, fpix=5. The relationship between absolute change and dimension is reflected in fuse length which changes for each point for each quantum change; but pretime change is a subset of this which reflects compressive and decompressive changes at different compression states which are perceived as force.

This sum goes 1, −2, −5, −8, −3, 2, 7, 12. One can see secondary dimensional effect, the initial folding leads to transitions on either side of the zero line in this case, a one dimensional line of solutions transitioning to a two dimensional display as collections of information represented by inflection points as well as the distances between these positive and negative peaks and the changing area within these. Moreover, this shows the beginning of dimension without a full collapse. Under the standard model, it was necessary to “invent” the concept of “energy levels,” beyond which electrons cannot go to collapse. In fact, it appears from this to be a geometry based separation, the accordion (shown in this figure) does not collapse because it can only be expressed in this fashion and being fractal, the universe follows this modeling at higher compression states and a type of emptiness which is not space between dimensional solutions is preserved.

The exponential area changes reflect that a circle increasing by 2× radius is exponential. CT1, ct2, and ct3 are designations to reflect compression, the association of fpix solutions “compressed” according to the equation 2f(x)\hat{ }(2\hat{ }x) where f(x) “begins” as the fpix equation (1, −3, 5, −7, 9 for x=1, 2, 3, 4, 5).

Where this gets interesting is between ct3 and ct4 where there are 16 primary sub-steps (2\hat{ }4=16). Measurements indicate that in base 10, 5.4 ct4t12 states form an electron. Some evidence indicates that this a base 14 system where 7 ct4t12 states would form an electron. In base 14, 14 ct4t15 states, form protons and neutrons while in base 10, 10 ct4t15 states would form the protons and neutrons. Historically, base 10 calculations were used, so much of the disclosure treats electrons in this fashion, even though it is likely a base 14 system as it appears that f(x) shifts from fpix to MI (Fibonacci) at the transition between protons and neutrons.

1) There is a quantum count (n=n+1). This happens to be the simplest iterated equation.

2) Fpix is ubiquitous because it yields the denominator of pi and curvature is ubiquitous. Since fpix is made of two separate iterated equations, it is safe to say it evolves from the quantum count. It uses −1 extensively. The quantum count has a counterpart n=n−1.

3) If you create a solution to fpix every time you increase the quantum count and use the quantum count to burn the fuse inherent in each fpix solution and graph the results you get a folded result. The use of the absolute value of the fuse may hide the source of overlap which a negative fuse could create. The nature of fractals (the results of changing iterated equations) is that they create repeating patterns. Fpix makes a convenient computer bit because we see it; and this technology builds on the creation of fuses and alternating positive and negative results, instead of 1 and 0 we have 1 and −1, or more precisely 1, −3, 5, −7, etc.

4) The folding creates compression in the sense that a one-dimensional string of solutions is folded into a two-dimensional string. Compression evolves or devolves. The compression equation 2f(n)\hat{ }(2\hat{ }n) where f(n) is the Fibonacci number (MI) for n works well at least for ct3 to ct4 compression. Fpix changes to MI between the proton and the neutron, so that f(n) in the equation above is fpix up to the proton and then changes to MI for structures larger than the proton. Two Fibonacci (MI) spirals constrained by 2\hat{ }n circles cause the two spirals to overlap with a ratio of 1:2:1. This neutron backbone then provides the framework for Proton-neutron bonding.

5) Inherent in the equations above is the evolution from fpix to 2f(n)\hat{ }(2\hat{ }n) AND the evolution of f(n) from fpix to MI and possibly to something after MI which we do not see because it lives in the heart of black holes.

Time is stop-frame animation based on changes in location pretime information states. Electrons are made up of photons in this scenario and the rotation of these photons (ct4t11 components) around electrons (ct4t12 composites) and even smaller pretime states (ct4t10, 9 etc., ct3 and ct2 states) around those photons gives rise to waveforms (Archimedes wheels show this) when viewed from the standpoint of time. Waves are pretime particles appearing in multiple places based on a post time perspective because we are using time, representing large numbers of pretime changes in a matrix, instead of quantum change common to the whole universe. Relativity doesn't exist for quantum change. Speed or gravity reflects externalization of these pretime quantum changes slowing the net change in the matrix, time dilation. At CT1, there is neither speed, time, nor gravity although the folding that results from folding ct1 into ct2 gives rise to quantum gravity. Life is the ability to use pretime change in a post time change compression state environment to effect pretime changes where they are useful.

The natural folding of FPIX solutions generates positive and negative peaks. These increasingly separate transitions mean that the universe goes from positive to negative constantly but over increasingly long-time scales. The big bang is nothing more than the last net compression changing at a single inflection point to net expansion. Each smaller unit (or matrix) of the universe does the same thing according to its own increasingly longer clock or fuse starting with each solution to fpix within that matrix.

The spinning of the ct4t11 pretime states around higher ct4t12 states is what gives wavelike features when viewed from the perspective of time.

Iterated equations are the results of prior solutions. A corollary is that more compressed iterated equations build from lower or less compressed iterated equations and hence you need these building blocks in sufficient concentrations called compression to get to the next higher compression state. Because less compressed fractals build from and therefore require lower compression states, clouds of lower states exist around more compressed states. Exceptions represent where the absorption and spew of the higher state is limited because of limited amounts of pretime change over an observed period. Vacuum depressurization reflects the inability to provide balance and the consequent spewing of information without a balancing intake.

Absorption and spew of information gives repulsion (and attraction) to magnets and creates positive and negative charge. It exists most critically at the transition between space and those things that we see in space like photons which also mark the transition between pretime and post time changes in information.

AuT targets things absent from the prior art: 1) time is a form of stop frame animation; 2) the difference between fpix (denominator of pi) and pi, 3) the absolute prominence of fractal math, 4) single base and multiple base math (for example base 10 and base 18) operating together simultaneously with transitions being transitions of state; and 5) the place where fpix transitions to MI. Others exist, such as a changing numerator for pi reflecting the building of dimensions from 1 to 4, the 4th not being immediately visible; but located at high levels of compression within large moons and planets as well as other ct5 (think black hole) oriented structures.

FIG. 2 shows scaling ct3 to ct4 transitions. T1 represents 10 (base 10) or 14 (base 14) ct3 states marking the first step in the 10-step transition to ct4 states, there represented by t16. T2 would have 10 T1 states (base 10) or 14 t1 states (base 14). The electron 12 is somewhere between T12 and T13 although it would have a cloud of lower states around it which is the case for each CT state. Time 255 arises between ct4t11 (T11) and the electron based on stop frame animation effects of all of the lower states as perceived from the standpoint of a time-based electromagnetic system of the type we experience.

The scales involved are comprehendible as fractal multiples. The transitions build according to an exponential increase whether 2\hat{ }n or a transition between 2\hat{ }n and x\hat{ }n the exponential result for x as a whole number greater than 1 means that there are exponential transitions between CT states. Continuity past ct4 is ensured since ct5 states, black holes are an observed example. Modeling indicates ct5 states are not just the large structures securing galactic spirals, but that they likely exist within, or at least existed within, spherical moon and planet bodies giving those their 3-dimensional features.

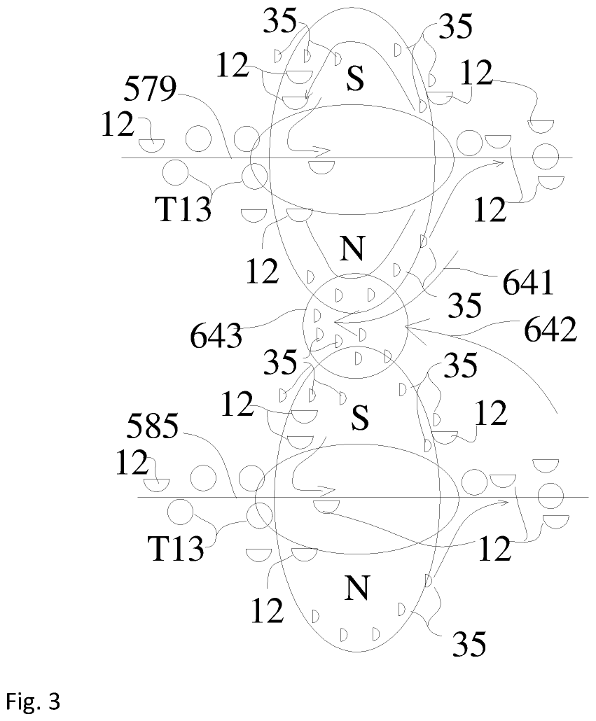

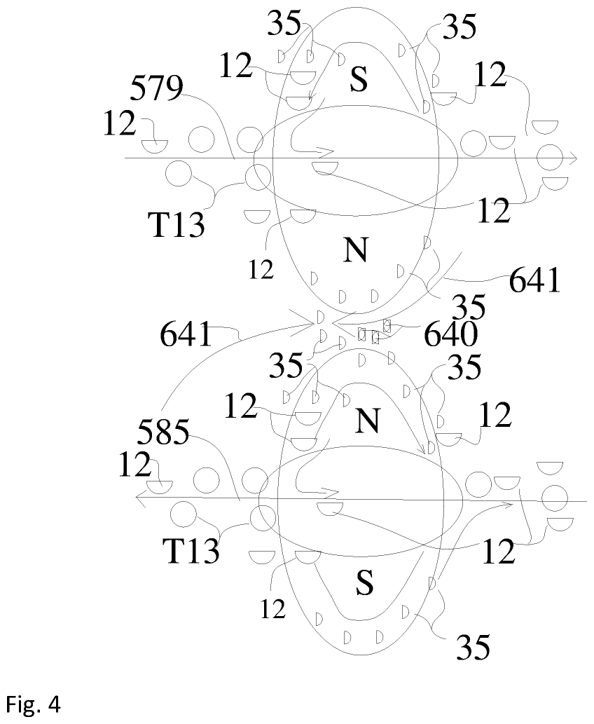

Hydrogen is not “an element” because it does not have a neutron backbone. Heavy versions of hydrogen do not have a neutron backbone, even though neutrons are present. A neutron backbone requires two neutrons share information, be balanced and stabilized by surrounding information states, namely protons and electrons. Modeling suggests that protons are ct4t15 states and a special type of positron which is one half of a ct4t13 state, the other half being the electron.¹ ¹ This eliminates energy, space and time in favor of changes to fpix and compression states related to fpix. Going back to the discussion of SE and Bohr, we are “quantizing” the wave form of electrons as 5.4 ct4t12 states (See FIG. 3 shows how electromagnetic fractal fields appear frozen between quantum changes and how magnetic attraction works. FIG. 4 is the same as FIG. 3 except it shows how magnetic repulsion works. We “see” pretime states as magnetic effects and space.

Time and pretime change are “frozen” in these figures. From a time-based perspective, the electrons pairs shown as t13 states would appear largely fixed about top wire 579 and bottom wire 585. Due to current through the wire, the movement to the left or right of electrons 12, a wire and being pretime these mechanical changes at ct4t11 gives rise to magnetic effects, the ct4t12 states are the electrical component. Being pretime, we only see the effects of the “magnetic” portion of the energy solution.

In both figures electrons are shown as t13 states T13, which are made up of t12 states as electrons 12. Circulating around the electrons 12 are M+ states 35 which are the magnetic ct4t11 equivalent of electrons which M+ states are shown rotated at 90 degrees and half size reflecting their exponentially information content (approximately 1/10^{th} that of an election) and their offset orientation which leads to the elliptical appearance of the cloud of information surrounding the top wire 579 and bottom wire 585. The amount of separation from the wire to the circulating M+ states magnetic field reflects the extent of the magnetic field portion of the current.

The M+ states rotate around the T12 states pretime and their positions are seen as waves as a result. If we take time out of the mix we can see that the two cooperating circulations in FIG. 3 make it relatively easy for the M+ states from the bottom wire 585 and top wire 579 to mix, exchange and the result, comparable to high and low pressure systems at scale is to draw them together. This is reflected in the area of sharing 643 In FIG. 4, since the path of rotation is opposite for the two flows of M+states between the top and bottom wires there are collisions 640 which lead to a buildup of these states which, from a post time perspective is seen as magnetic repulsion. The pretime change inherent in the M+ states are what powers the ct4t12 states as they not only circulate but exchange information. Because the ct4t11 states are pretime we only see the net effects as repulsive. Our ability to get post time “work” from pretime “change” is the energy effect.

Several factors affect the wave pattern, the distance from the wires of the flow of information which may be increased and disturbed by coiling the wires as is known in the art of generating magnetic fields. Another factor is the amount of pretime change in the M+ states which can be thought of as the number of revolutions about the t12 states over any period of time. Increasing the speed of rotation, the movement pretime, results in a shorter wavelength. The more the wheel changes within a given distance, the shorter the wavelength; hence the more “pretime” changes, the shorter the wavelength and the more energy in a ct4t11-12 matrix.

The figure shows frequency generation from a particle moving along a pretime length creating a wave effect when viewed from the standpoint of time and where “energy” is the ability to use this pretime change in a post time change environment to do work and to provide, from the perspective of time “increased space” or “increased pretime change” viewed as energy within the framework on the left expanding the apparent space occupied by smaller particles when viewed from the standpoint of time. 5.4/7; 5.4/14 base) where the 0.4 reflects the ct4t11 (and lower) CT states in the electron matrix. Varying the number of these pretime CT states (each ct4t11 is a quantum photon element, although the accumulation of ct4t11 states into a photon likely tracks the number in an electron with a corresponding 0.4 cloud of ct4t10 states) in an electron matrix of CT states, we vary the energy of the “average” electron. This is complicated because of varying net rates of change for electrons, but these differences are homogenized for most observations since even a single amp involves the measurement of 6.24×10\hat{ }18 electrons.

Energy is pretime change; frequency is energy reflected as the number of pretime changes in ct4t11 rotation about ct4t12 seen from the viewpoint of time; Planck's constant is the size of the ct4t11 states which are subsequently viewed as energy from the perspective of time in rough terms. Plasma and plasma control can be defined by the mix of fractal information of a particular type within a defined matrix much like an airplane can be buffeted from a time-based perspective by changing winds. Knowing these circulating structures exist they can be used to move and empower transitions in higher CT states.

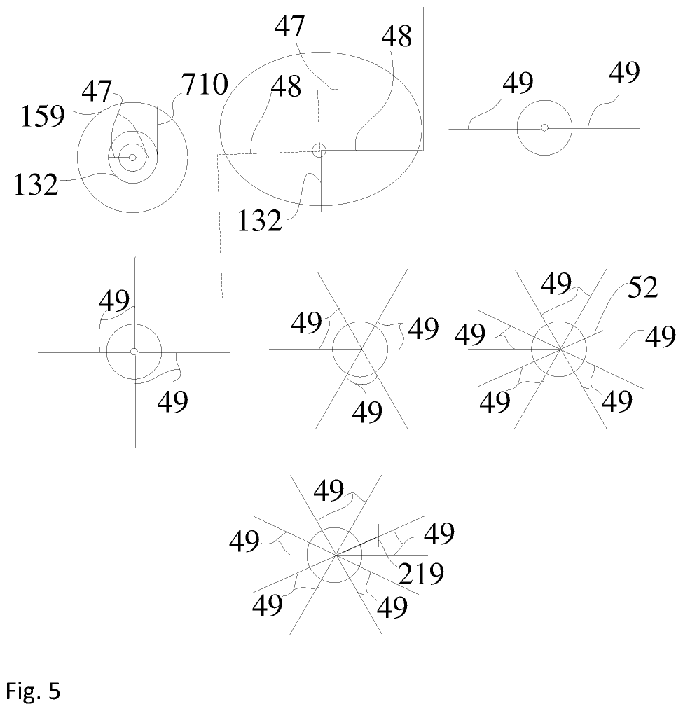

FIG. 5 shows proton charting with balance based on fpix and balanced solutions. The fpix solutions are 1, −3, 5, −7, 9, −11. These are the only solutions applicable. The 2 inner arms each have a value of 1, then there is the negative value of −3, and for the next arm and so on.

The Fractal origin of Proton and Electron Counts shown graphically based on 2f(x) where f(x)=fpix reflecting the balancing (the factor of 2) critical to the structures defined; 2*1=2, 2*−3=−6, 2*5=10, etc. The “even” results give rise to the noble gases, the “odd” results define the semi-conductors.

Referring to FIG. 5, there are the two balancing hydrogens 47 for a helium atom 132 here identified by the effective radius of the proton core. Also shown are the 3-length negative value arms 710. There are two of these waiting to be filled to give Carbon which area is identified by the potential carbon core radius 159. Next is Neon which has 5-unit balancing arms 48. Items 47 and 132 are shown for reference. Next is Argon where the scale has been changed. The negative value arms −7 are not shown, but the sum (14) would yield the next semiconductor, Silicone. For Argon there are two balanced arms 49 giving the 18 proton total observed. After Argong, there are 4 9 unit arms 49 for Krypton, 6-9 unit arms 49 for Xenon and for Radon there are 9, 9-unit arms plus a broken arm which is 5 unit unbalanced arm 52. While shown this way for clarity, balance suggests that the broken arm and one of the 9 unit arms 49 would each be 7-units and balanced, although different from the 9 unit arms. Each of these 7-unit arms (not shown) might come off of one of the 9 unit arms, by way of example. The repeating “9's” indicates that the repeating feature of the neutron backbone is the that shown for Argon (2×11=22) as opposed to the balanced “5's” of Neon although radon indicates it might be the balanced 5's with a bridge of 2×1=2 between the two neutron backbones. Also shown as an example of larger proton balancing is Uranium which is shown with 10 9-unit arms 49 and at the place where the unbalanced arm 52 would have ended there is a helium proton pair 219. The relevance of this modeling will be discussed in more detail.

The broken Radon may correspond fractally to the electron-proton interface.

### Electron Charting

Electron “full orbitals” are 2 times the absolute value of fpix (1, −3, 5, −7, 9). for fpix values of n=1 to 4. This is two times 1, 3, 5, and 7. The electron count (full shell) matches 2×fpix solutions 2*fpix gives full electron orbitals (2, 6, 10, 14) just like the Proton count for noble gases reflect 2×fpix solutions, skipping the negatives.

The proton counts for noble gases reflect 2 times the positive fpix solutions (namely, 1, 5, 9 for Helium, Neon, and Argon. The next noble gases proton counts are multiples of 9 for Krypton (4×9), Xenon (6 times 9), and Radon (10 times 9, but with 4 less protons showing the importance of the neutron backbone at these scales). These transitions reflect the positive fpix values up to Argon and then build from the negative fpix values. The initial “noble” change is 1 (2 since there are two spirals); then 5 (10); then 9 (18) skipping every other result in the rush towards noble relationships for the proton count. The 3(6) and 7(14) remain relevant as semi-conductors, shells filled to give stabilized repeatable, connectable with efficient electron state exchanges; the 3 in particular as the 6 unit/carbon-like ring structure.

In fractal modeling, there are no electron orbitals as such to fill. Electrons are associated with and balance the protons in an atom. Balanced layouts and the other fractal features may be targeted for fusion, fission, chemistry, electromagnetics. Dissolving this balance between the AuT position and electron gives rise to ionization. Electron orbitals are discrete due to discrete ct4t12 or ct4t11 information exchange. Lower states being too small to show up being 14\hat{ }100th smaller than the forces we use.

Fpix modeling with N=ct4: If we use f(x) for ct4 is 7, then we have a base 14 compression equation. To get an electron of consequence we can use the ratio of ct4t12:t16 which is 2.6×10\hat{ }−5 meaning the electron is 20.89 ct4t12 states which is robust electron compared to model using 5.4 ct4t12 states (if it were in t13 states it would be 1.5 t13 states). Transitions are base 14 transitions between ct4t12 and ct4t13 and there is no clean transition between 20ct412 states and ct4t13; you already have 1.49 t13 states within the ct4t12 states. Such a “two times” 20.89 ct4t12 unit; ct4t13: 41.78/14=2.98 ct4t13 states.

This may be a balanced 1.48:1:48 product compared to a 1.005:1.005. Much of the information is “massless” because it is in the form of energy equivalent pretime states.

The pair would absorb ct4t11 (or ct4t12) from a source which would give it a charge type exchange (positive or negative).

The ct2:ct5 ratio for gravity to the strong force works well in this scenario. (1296:1.47×10\hat{ }40 =1×10\hat{ }37). If the initial ratio is used, it is 14 or 196:1.47×10\hat{ }40, the 196 number working well). We are working in base 7 or if balance is included, base 14. There are 1.49 T13 states and, multiplying this by 14, there are 20.9 ct4t12 states in base 14 fpix mathematics, 3.84 times as many ct4t12 states as seen with base 10 math.

A pair of electrons under this fpix analysis would be 41.8 ct4t12 states or 3 ct4t13 states less 0.015 lower information states. One might alternatively call it 2 ct4t13 states plus a cloud of almost 1 ct4t13 state's worth of ct4t12 and lower information states. Keeping in mind that this is a base 14 system, this lower information represents 7.03 ct4t12 states.

If we look at what happens if we ignore the 0.4, the shadow of lower CT states around the 5 ct4t12 states, instead of 5.44×10\hat{ }−4², it is 5×10\hat{ }−4 and the resulting measurement (0.0005×14) is 0.007. When you average these two results you get 7.30716×10\hat{ }−3 as the electromagnetic effect, an average between the pure ct4t12 and ct4t12 plus lower state effects. This is 99.8657% the same result using the non-fractal morass of equations used to calculate the fine structure constant (FSC).

The problem with shifting base numbering is that we have to derive it from observations consistent with the modeling. Full ct4t12 pairing gets a ct4t13 in base 10 may be cleaner than a pair of t13 states, but we also have the evidence of Helium equivalence to paired t13 states. Full shells appear as t13 pairs in the 1.45:1.45 analysis. The evidence includes the resulting similarity of the electron to the fine structure constant and fpix pairing numbers. This is the process of using the fractal design, categorization and use of features which we have.

The electron viewed as paired T13 states with clouds of ct4t12 (and lower) can find a fractal counterpart in paired neutrons in helium. This is not definitive, but there is support in the modeling.

Neutron MI spiral folding reflects the overlap where fpix folding gives way to MI overlapping spiral folding yielding solutions in the “calculated” column below. The Fractal origin of Neutron count as a transition to a base 10 system with folding restricted by fpix 2\hat{ }n circles shown below by measuring the inflection points (lengths measured between 2\hat{ }n and spiral arms intersections or spiral arm direction changes).

| | calculated | | | | |
|---|---|---|---|---|---|
| observed | running | observed | | | |
| neutrons | total | protons | Element | fpix | |
| 2 | 2 | 2 | He | 1 | Overlapping spiral inflection points |
| 6 | 5.499093 | 6 | C | 3 | (intersection with 2\hat{ }n cricles or change in |
| | | | | | fpix) yield the resulting atomic/post atomic |
| 10 | 9.824209 | 10 | Ne | 5 | MI structures and reflect the transition from |
| 14 | 14.12121 | 14 | Si | 7 | fpix related structures and MI related |
| 22 | 22.79091 | 18 | Ar | 11 | structures. The initial overlap is 1:2:1 |
| | | | | | allowing the remaining numbers to be |
| | | | | | calculated using simple trigonometry. |

If you measure the length of the spirals which have been confined by the Fpix defined circles, the lengths yield the neutron count for the first 3 lines of the PTE. Multiple other features of atoms are apparent, and the F-series ratio of protons to neutrons established are maintained for all subsequent atoms. The initial “noble” change is 1 (2 since there are two spirals); then 5 (10); then 9 (18) skipping every other result in the rush towards noble relationships for the proton count. The 3(6) and 7(14) are relevant reflecting semiconductors; the 3(5) as the 6 unit/carbon-like ring structure. ² This is actually 5.438806×10\hat{ }−4 in the calculation and is the measured mass of electron divided by the measured mass of the neutron which is complex because much of the information stabilizing the electrons is considered without mass.

Visible transition from Fpix to MI in the equation 2f(n)\hat{ }(2\hat{ }x) occurs at the proton to neutron fusion location and is visible in atomic and post atomic structures.

The MI overlapping spirals are reflected in the appearance of MI in large structures from atoms to galaxies as shown with the example above right. The model shows shifting base numbering complicating an already complex compression and decompression (folding and unfolding) mechanism. Fractals give rise to curvature (accepted science, although not with this breakdown of elements) and fractals give giving rise to the electron shells as well as proton and neutron counts.

### Summary List of Proof

There is no set number of coincidences before models transition from theory (or in this case math model) to fact. While never a certainty, modeling support for using fpix and exponential compression is easy to document:
1) The proton count (at least for noble gases and semi-conductors).
2) The electron count for full electron shells.
3) The neutron Count and the 3:5 (example) ratio reconciling the count of P and N.
4) Relative strength of gravity to the strong force based on the compression equation (2f(n)\hat{ }(2\hat{ }n) for n=2 and n=5). This follows the idea that forces arise from folding and unfolding of CT states.
5) Overlapping MI spirals and 2\hat{ }n compression appear in post atomic structures.
6) Generation of the fine structure constant without relativistic or non-fractal elements. This is a complex discussion, but you can approximate the result by multiplying the electron/neutron mass ratio (5.44×10\hat{ }−4) times 14 (14 ct4t11 states per ct4t12 electron). FSC reflects ct4t12 fractals folding.³
7) Fpix gives rise to pi. While this is the origin of our examination of fpix, we cannot take curvature for granted. The numerator is also suggestive, reflecting an n=n+1 equal to 4 showing a neutron level curvature based on n=4. Curvature does not need to be based on fpix, it could be anything, even a non-iterated equation, it could be based on a fixed number (like the approximation 22/7), but it is not.
8) The math can be applied and seen in action at all levels of dimensional change.

Theory also supports the model. AuT (the theory) provides an explanation for:
9) Why neutrons dissolve when separated from the atom (because there is insufficient balancing (absorption material) the neutron spews out its existence as information which would otherwise circulate in the system. The proton appears stable when isolated, but it is not isolated from other base 14/fpix states. Neutrons must be stabilized by MI (base 10) states, hence the reason for neutron pairing in stable atoms. Heavy water likely results from Neutrons with low pretime change rates at MI, although they can still have fpix state transitions.

³Results may be coincidental, but quantity and in some cases quality of results justifies this investigation. No other math model moves from physics through post atomic structures using just ubiquitous fpix. Further, base transitions involve exponential (x\hat{ }n) results, 2\hat{ }n in the case of the transition from ct1 to ct2 (base 1 to base 2).
10) A definition of quantum gravity (folding of ct1 states into ct2 states).
11) Where folding of information originates, FIG. 1.
12) Electron pairing as overlapping spiral and electron-positron bonding showing protons and electrons are necessary for stability (sharing absorptions and spew keeps them from dissolving like the neutron);
13) A definition of time as stop frame animation based on pretime (pre ct4t12) dimensional change.⁴
14) Wave Particle duality defined, pre-electron particles (ct4t12 and smaller states) change in a pretime environment, literally in multiple places at once from the standpoint of time.
15) Energy: post-time life taking advantage of pretime changes to do work. work in post-time is life taking advantage of pretime changes to do work for the perspective of time.
16) A definition of space at the pre-energy dimensional states.
17) A definition of electromagnetism and magnetic repulsion as high- and low-pressure type interactions based on the net appearance of pretime states (ct4t11) circulating around ct4t12 electrons seen from the post time perspective.
18) A definition of dark energy based on the net decompression of the universe which will eventually be replaced with net compression.
19) Dark matter—net spiral compression.
20) The current expanding universe (based on net decompression and to some extent the addition of new bits of information) and its eventual partial compression before another round of decompression (net).
21) An origin for dimension. Fpix naturally folds (reflected in curvature) from 1 to 2 dimensions shown in the “All the math in the universe” slide with the folding 1 picture.
22) There are additional problems with the standard model which do not exist in fractal mathematics (eliminating “free parameters” and “force carriers”, for example).⁵
23) The appearance of the overlapping spirals repeatedly in post atomic structures. A new model, verified visually, for the appearance of overlapping spiral configurations reflecting compression which can be seen at atomic and post atomic scales along with large scale 2\hat{ }n exponential circles (galactic ng2775 for example).

These are all based on accepted measurements. Theoretical results also support the model.
24) Spew without absorptions explains why neutrons dissolve when separated from the atom (because there is insufficient balancing, absorption material, the neutron spews out its existence.
25) Why protons and electrons are necessary for stability (sharing absorptions and spew keeps them from dissolving like the neutron) and the exchange or ability to exchange ct4t12 sates gives rives to charge.
26) The answer to Zeno's paradox at the subatomic level.
27) A grab bag of problems with the standard model that do not exist in fractal mathematics ⁴This definition of time also explains the apparent instantaneousness of “net” gravity, all of the folding creating gravity and unfolding (anti-gravity) occurring pretime like magnetic repulsion, but more so.⁵The Standard Model is incomplete. Quantum field theory is an effective theory, but many believe something fundamental lurks beneath. The Standard Model does not incorporate gravity. The Standard Model doesn't explain the three generations of fermions and has as many as 26 free parameters, the values of which are not predicted by the theory, arguably a clumsy framework of different symmetries. AuT has a single evolving symmetry (fpix) giving rise to folding and exponential results and all force reflects folding (or unfolding) of information states eliminating both “force carriers” and whatever they are supposed to carry.

### Compression Matrix

Simple Compression according to Fpix

| | | | | ct1 | Observed |
|---|---|---|---|---|---|
| n | f(n) | 2\hat{ }n | compression | states | part |
| 1 | 1 | 2 | 4 | 4 | Pre-dimensional |
| 2 | −3 | 4 | 1296 | 1296 | space 2 |
| 3 | 5 | 8 | 1E+08 | 1.3E+11 | space 3 |
| 4 | −7 | 16 | 2.18E+18 | 2.82E+29 | Magnetism-neutron |
| 5 | 9 | 32 | 1.47E+40 | 4.16E+69 | Molecular-black hole |

There is substantial evidence that compression before the neutron does not involve the Fibonacci series (hereinafter “MI” referring to the Indian Method) which otherwise looks like this:

| | | | ct2 states | |
|---|---|---|---|---|
| | | | changing | 2f(n)\hat{ }2n |
| N | 2*f(n) | 2\hat{ }n | ratio | compression |
| 1 | 2 | 2 | 4 | |
| 2 | 4 | 4 | 256 | |
| 3 | 6 | 8 | 1679616 | 1679616 |
| 4 | 10 | 16 | 1.68E+22 | 1E+16 |
| 5 | 16 | 32 | 5.72E+60 | 3.4E+38 |
| 6 | 26 | 64 | 2.1E+151 | 3.62E+90 |

Because fpix naturally progresses from one dimensional results to two dimensional fold and because this involves a base change, 2\hat{ }n is a potential part of the transition as the exponent in f(n)\hat{ }(2\hat{ }n) and/or an intermediary state in fpix evolution, possibly leading to the result of 1,2, 3, 5, 7, 9; partially mimicking MI and leading to this series.

| n | f(n) | 2f(n) | 2\hat{ }n | compression |
|---|---|---|---|---|
| 1 | 1 | 2 | 2 | 4 |
| 2 | 2 | 4 | 4 | 256 |
| 3 | −3 | -6 | 8 | 1679616 |
| 4 | 5 | 10 | 16 | 1E+16 |
| 5 | −7 | −14 | 32 | 4.74348E+36 |
| 6 | 9 | 18 | 64 | 2.17491E+80 |
| 7 | 11 | 22 | 128 | 6.7624E+171 |

If force reflects net folding, then the force of gravity can be reconciled with the strong force by compression moving from n=2 to n=3 according to fpix compression and then transitioning at the proton:neutron interface to the MI compression equation.

### Base Transitions

Fractal modeling can be used to model the complex and intricate structures of particles and to predict the behavior of charged particles. Fractal models are useful for modeling systems that are difficult to describe using conventional mathematical techniques because they can reduce complex and irregular shapes and structures found in these systems to fractal components.

Base transitions involve exponential (x\hat{ }n) results, 2\hat{ }n in the case of the transition from ct1 to ct2 (base 1 to base 2). Likely 2\hat{ }n arises from this base number change reflected in the 2 unit difference between solutions to fpix. This raises the possibility that the exponential 2f(n)\hat{ }(2\hat{ }n) equation might look more like 2f(n)\hat{ }x where x varies and the possibility that various exponential 2\hat{ }n, 3\hat{ }n, 5\hat{ }n, etc. might come into play in the mathematical compression equation where n is equal to a number or an equation (like 2\hat{ }n or 3\hat{ }n or some variation of those, including something like (5\hat{ }n3)\hat{ }(3\hat{ }n2)\hat{ }(2\hat{ }n1)).

The transitions observed between electrons, protons and neutrons and energy appear as 2\hat{ }n, at least at energy from matter transitions.

Coincidence is often viewed as a random event, something that occurs without any “math,” other than probability which is inapplicable at a quantum level. Everything that happens is a result of a cause-and-effect chain, originating dimensionally in a series of solutions to the equation fpix changing based on n=n+1. The idea of coincidence is a human construct, an attempt to explain hidden patterns. AuT sets out the patterns, patterns not hidden, their significance overlooked.

Pre-ct4t11 changes are the pretime, stop frame animation changes that create the dimensional effect we call time. Note that acceleration does not create relativistic effects at the quantum level because (1) there is no time and (2) all changes occur in ct1 states at the same rate, although staggered by fuse length.

### A Look at “Rays”

A broad view of fusion based on collapsing balanced Helium atoms, getting a balanced atom with proximities is the combination of collapsing or reorganizing protons into neutrons and then balancing them so the net energy release is maximized.

Alpha, beta, and gamma rays are three types of ionizing radiation. Alpha rays are particles that are made up of two protons and two neutrons, which are tightly bound together, the extracted pair of protons from the w extra protons modeled from the 4-sets of 9 plus 2 proton core fractal model of 78 protons required by AuT modeling (FIG. 5). They are positively charged and are relatively heavy. Because of their large size, alpha rays are not penetrating, and can be stopped by a sheet of paper. Beta rays are high-speed electrons that are emitted by certain radioactive isotopes. They are much smaller and more energetic than alpha particles and can penetrate deeper into matter. Gamma rays are high-energy photons that are produced by the decay of atomic nuclei. They are the most energetic and most penetrating of the three types of radiation. Gamma rays are not called particles, but rather electromagnetic radiation, similar to light and X-rays. What makes Alpha and Beta into “rays” and not just their constituent parts are the increased “field” effects, what AuT would call the amount of pretime change incorporated into the matrix of CT states defining them. While Alpha and Beta can be thought of as collections of ct4t12-ct4t15 states; Gamma rays can be thought of as a matrix of ct4t11 states.

AuT allows for all states, energy and matter and transitions between them and even space to be viewed as frozen moments.

### Plasma

Math modeling is essentially programming and reducing plasma interactions to mathematical changes allow Fractal modeling to be used to address plasma and plasma controlling devices. Plasma is relatively easy to model as ct4t12 states dissolved in a fluid of ct4t11 states which allows for the separation of electrons which are otherwise bound by protons viewed as 10 ct4t15 states, one of which has a ct4t12 “AuT positron” (A-positron) extending from the ct4t15 which A-positron overlaps with a ct412 electron to form a stable proton-electron pair (ct4t13). Balanced fractal layouts and information exchange may be targeted for fusion, fission, chemistry, and to study plasma.

### Key Fractal Elements (KFE)

Broadly speaking KFE is just the math set out and the ability to refine it. Application of KFE is using KFE to affect change in CT states, interpret or categorize matrices of CT states. The process is applying KFE elements for categorization, prediction and manipulation and to design radiation matrices for electronics, solar, thermal, fusion and radioactive energy use, capture and dispersal; KFE used to modify frequency-based systems increasing the efficiency of those systems for generation of energy, transmission of energy, use of energy and storage of energy as well as increasing the overall accuracy of results.

Base transitions governing matrix composition and interaction including spatial, energy, atomic, chemical, electrical, biological and large structures are also key fractal elements as observed not only for protons and neutrons, but also in lower and higher CT state matrices. Base transitions are those base numbering systems resulting from AuT fractal math being applied to increasing and decreasing compression.

KFE are used to compress these or decompress CT states and to form matrices of CT states. An example is the transition from M+ct11 states, ct12 or electrons in the case of energy. The application to energy is the ability to channel pretime change where it can be used; targeting different dimension and base numbering and information exchange of different CT states based on changing base numbering and resulting dimensions.

KFE includes CT states, fpix as an equation, fuse length for individual solutions to fpix as well as for matrices of solutions and compression states, MI, MI defined spirals, overlapping and exponential compression features, particularly as functions of fpix and MI, changing vs stable pretime features, the nature of pretime change vs time-based change, the relative amount of pretime change vs non change in a matrix, changes in the quantum count, KFE spiral alignment, pretime change CT state changes, CT state sharing and the related concept of collisions as information exchange, net compression/decompression of CT states and matrices and the related force or energy features, balance and imbalance, changing base numbering reflected in CT states and dimensions, and net pretime change characteristics or a combination of those features. KFE can be used in field modeling and mechanical interactions.

KFE includes changes in KFE and KFE energy converters can be used for staged fuels with fpix, MI and exponential changes such as 1:1; 1:2: 1:3:1:5 for burn times, heat, pressures, mixtures, steps and reactant matrices beginning and ending for any reaction to target specific fractal results.

KFE includes the fractal design of matrices using time as CT state dimensional change, energy as CT state change, the Neutron backbone, the structure of resulting neutron backbones, proton cores and electron clouds as pathways to chemistry, to increase and control the release of energy, redirection or absorption of the energy. This includes fractally relevant shapes such as six or 14 sided structures representing atomic inflection points and pre-atomic, proton inflection points respectively.

KFE includes designing, based on KFE concepts, of molecules, the dimensionally expansive or contractive features and the orientation of atomic or molecular matrices created, the order of creation, and orientation based on KFE in matrices at all stages of reactions as CT state matrix change.

KFE includes and enables dimensional variations in a matrix and the benefits that come with work with dimensional variation.

KFE includes control of where absorption and spew are generated within matrices of CT states and where it is discharged into matrices which includes design of machines, reactions using KFE.

KFE “engines” focus on absorption or spew of CT state exchanges at focused locations along with the use of this focused exchange.

KFE includes designing, based on KFE, the proximity of states and the nature of the intervening matrices between reactant matrices, rate of injection and alignment by shaping of the reaction chamber parts (cylinder, piston, injectors); the number, location, and series of injector jets and exhaust and by controlling the mixtures of different CT states used to maximize desired matrix results, for example to maximize the heat energy going to expansion, contraction or other desired post information exchange matrix.

Reacting chemicals, including fuels, using KFE can get more efficient matrix changes and maximize the release of pretime informational change includes spiral/exponential/centrally destabilized pathways; maximizing mining ct4t11 trapped in a matrix including stabilizing the resulting matrix. One example is using absorption and spew designs to flush high pretime change from a matrix with low pretime change ct4t11 to balance the release of the high pretime change ct4t11.

KFE can be used to change the contents of the fuel during the burning process, the timing of the burning process, volumes, shapes and the use of the energy states based on KFE.

AuT allows for time to be taken out of the equation and for focus on how to use KFE to maximize the amount of pretime change to maximize the type and location of pretime states separating and combining with atoms and molecules to control pressure, energy and reactants generated.

KFE includes targeting AuT plasma acting as fulcrums and associated stepped transitions, categorization, chemistry, biology, and other features.

Balance is shown where a plasma fulcrum is the central most neutron-to-neutron plasma fulcrum of shared information between those central most neutrons, everything else balanced and folding around the fulcrum; balanced by absorption and spew between the elements. Added or inherent to these, is the fractal form in balance (the linear spirals), plasma centers between CT states, and net absorption leading to the folding. A fulcrum of lower states about which higher states balance, fractal alignment on either side of the fulcrum occurs because higher CT states form within shells of folding lower states. Balance and folding around the fulcrum are by absorption and spew between the neutrons and then between the protons and neutrons and then between the electrons and protons. This fulcrum of lower CT states continues, including the effects of magnetic repulsion and their corresponding effects of high- and low-pressure system interactions.

### Grouping of Fractal Effects of Fulcrums and Balance

### Key Fractal Elements Observed:

CT states and their manipulation and interpretation.
Base transitions governing matrix composition and interaction in various domains.
Compression and decompression of CT states and formation of matrices.
Fractal features such as spirals, overlapping, and exponential compression.
Pretime change and its relationship to dimensional variation.
Spiral alignment and its impact on CT state changes.
CT state sharing and collisions as information exchange mechanisms.
Net compression and decompression of CT states and matrices.
Balance and imbalance within fractal structures.
Changing base numbering and resulting dimensions.
Pretime change characteristics and their effects on matrices.
KFE's role in field modeling and mechanical interactions.
Energy converters based on KFE principles.
Designing matrices and structures using KFE concepts.
Control of absorption and spew within matrices of CT states.
Focused exchange and its utilization in KFE engines.
Control over fuel composition and timing using KFE.
Maximizing energy release through fractal pathways and shapes.
Designing molecules and atomic matrices based on KFE principles.
Utilizing dimensional variations for improved matrix outcomes.
KFE's role in proximity and intervening matrices in reactions.
KFE's Impact on reaction chamber design and performance.
Maximizing efficiency and results in fusion, fission, and chemistry.
Fractal design of matrices for electronics, batteries, and energy systems.
KFE's influence on quantum computing and AI algorithms.

These elements represent key observations and principles related to fractal phenomena and their application in various domains, as described in the disclosed information. It is helpful to think of KFE groups.

### Group 1: Fractal Elements of CT State Transitions

Stepped AuT fractal transitions are defined as fractal transitions governing CT state changes. MI overlapping spirals of CT states in and out of alignment and related absorption and spew between matrices.

Pairing of higher compression CT states along f-series linear spirals, curved spirals and shared lower compression CT states.

Folding to get compression and unfolding to get decompression along fractal linear spirals of CT states about at least one AuT fulcrum of “shared” lower CT states, shared meaning that over time relevant quantum changes CT states from the fulcrum are exchanged with the higher CT state matrices on either side.

Compression as lowering lower compression CT states between higher compression CT states and decompression as numerically increasing lower compression CT states between higher compression CT states.

Fulcrums as AuT plasma centers between CT states defined as the areas between CT states where lower CT states are shared by higher CT states along KFE rules of compression.

Net compression or decompression as force when observed from the standpoint of time.

CT states defined as stepped fractal dimensional states from iterated equations defined by fractal compression or decompression due to compression of lower CT states along KFE rules of compression. Force defined as the result of net winding or unwinding of CT states as viewed from post-time CT state perspectives including ct4t11 pretime changes viewed as energy.

Time is defined as stop-frame animation resulting from changes in pretime CT states defined as CT states at and below the level generating electromagnetic effects.

Relativistic effects as the difference between pretime and time-based change

Curvature defined by a solution to fpix for pi with definitive limitations generating the sequential amounts of dimension and curvature in response to net CT state compression.

### Group 2: Elements Related to AuT Matrices.

Base state changes are inherent in CT state folding.

Fractal balance, defined as alignment of at least two higher compression CT states about at least one

AuT fulcrum comprised of lower compression CT states by way of sharing the fulcrum.

AuT fulcrums are defined as lower compression CT states at the overlap of compression of at least two higher CT states; where a ct4 state neutron backbone is part of the higher CT states to form atoms. Absorption can increase or decrease compression of a matrix to higher CT states. It depends on the net compression of the matrix at the two points of observation. The same is true about spew of lower states from within a higher CT state compression matrix.

Using KFE to define “base logic,” mathematical “base” term use, to categorize at least one AuT Matrix.

Features include: net compression as the net compression or decompression within an AuT matrix; shifting between higher and lower compression of CT states within the AuT fulcrum; Absorption and spew between at least the first AuT matrix and at least one second AuT matrix which is a little different where the second matrix includes the first within it; CT state exchange between at least two AuT

### Matrices in Place of Collision or Field Modeling

AuT matrix categorization using 1) CT state content, 2) amount of CT states within the content; 3) relative dimensional size to at least one second matrix, 3) locational area from the perspective of time of generated by pretime change of the at least one matrix; 4) AuT plasmas; 5) fulcrum locations; and the amount of pretime change of the CT states, compression and decompression tendency within a matrix.

Basing thermodynamic effects of at least one AuT matrix based on categorized CT states within the at least one AuT matrix.

### Group 3: Absorption and Spew of CT States

### Treating Exchange of Lower CT States Between at Least Two Higher CT State AuT Matrices as the Source of Interaction

Collisions as the exchange of information between at least two AuT matrices and post-collision effects reflecting the net change of CT state and pretime change in each matrix of the at least two matrices. F-series spirals of CT states in and out of alignment for absorption and spew

### Group 4: CT States and Fractal Elements

CT states are defined as stepped fractal dimensional states from common iterated equations defined by fractal compression or decompression due to compression of lower CT states along f-series fractal lines. Force defined as the result of net winding or unwinding of CT states as viewed from post time CT state perspectives including ct4t11 changes viewed as energy.

Time defined as stop frame animation resulting from changes in pretime CT states defined as CT states below the level generating electromagnetic effects; relativistic effects as the difference between pretime and time-based change.

Fuse length as a fractal element of CT state transition from compression to decompression. Curvature defined by a solution to fpix for pi with definitive limitations generating the sequential amounts of dimension and curvature in response to net CT state compression.

Fulcrums as shared CT states between higher CT states.

Quantum fractal dimensional change in place of divisions of time which is more fluid since it is an Effect of quantum changes at a relatively high level of compression.

Group 5: AuT Matrices and their Categorization

Using AuT as “base logic” of at least one AuT Matrix.

Net compression as the net compression or decompression along a fractal pathway mathematically derived from fpix, quantum changes in solutions to fpix, and ultimately the components of fpix within an AuT matrix.

Shifting between higher and lower compression of CT states within the AuT fulcrum

Absorption and spew between at least the first AuT matrix and at least one second AuT matrix

AuT matrix categorization using 1) CT state content, 2) organization and type of CT states leading to compressive features within the matrix; 3) locational area from the perspective of time; 4) energy level generated by pretime change of the at least one matrix; and 5) fulcrum locations and categorization. This includes basing related thermodynamic properties of a first AuT matrix with at least one second AuT matrix based on the categorized matrices and intervening CT state matrices.

### CT State Exchange Between at Least Two AuT Matrices in Place of Collision or Field Modeling

Visible at the Proton-Neutron interface is balance of f-series linear spirals, net absorption leading to the folding or closing in of the spirals around the areas of overlap represented by fulcrums which incrementally increases the compression.

Imbalance using lower information states. The direction and form of winding and to maximize CT state exchanges to transfer information and maximize work.

### Electromagnetics: Category Specific Science—

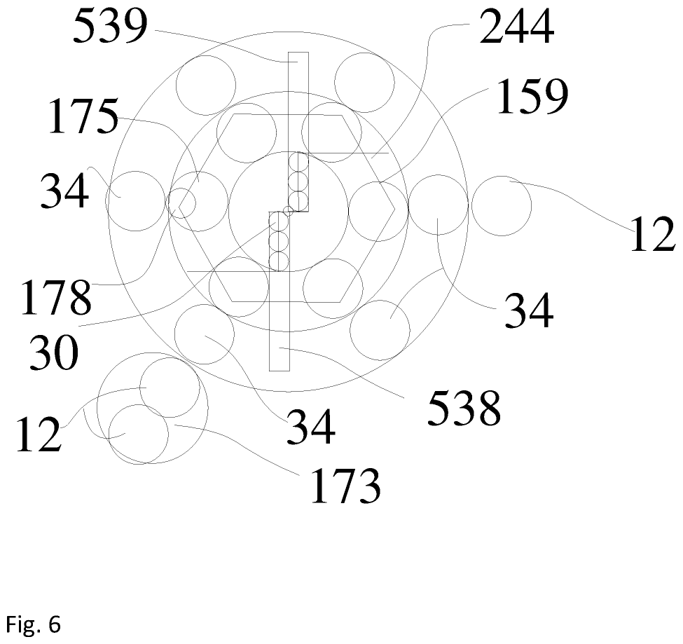

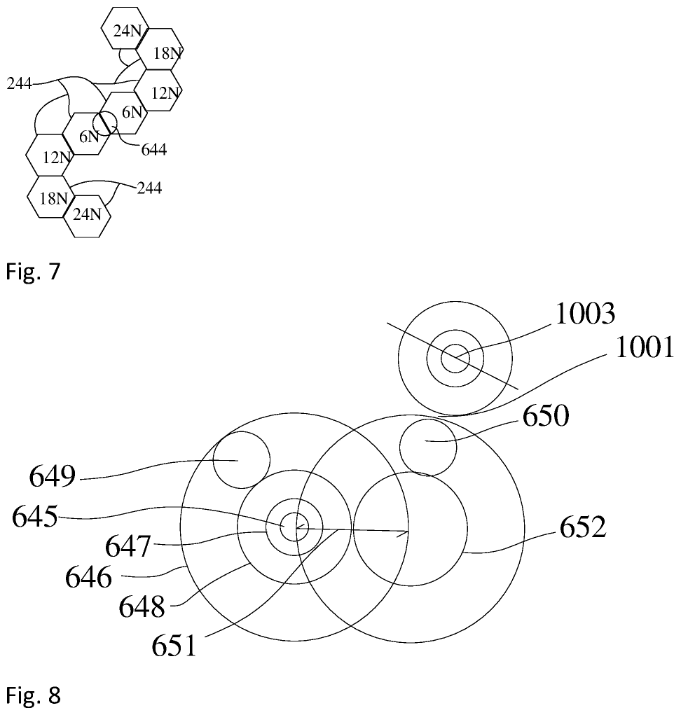

FIG. 6 shows how atomic structure allows for exposure of outer “electron proton pairs” allowing the electron-positron bonds of an atom to be extended along fractal lines through electron pairing which provides stabilizing fulcrums on either side with positrons on either side of the protons paired for circulation of CT states and balance. As shown in FIG. 6 there is a copy engine 539 and bottom engine 538 each of which contain 3 neutrons 30. The spirals that define the location of the six neutrons and this carbon equivalent are not numbered for clarity as this information is covered in previous patents.

Outside of the circle defining the location of the neutrons is a second circle which defines the six sided structure 244 of the balancing protons 175 within each of the protons is an unbalanced T15 state 178 which allows the sharing of information with a positron 34. Each proton 175 has a corresponding positron 34 the positrons 34 are balanced with either an electron 12 or by an electron pair represented by a T13 state 173.

This shows eight 6-sided structures 244 about a common neutron fulcrum 644. This forms a total of 48 neutrons identifying it as the neutron core of the primary isotope of Krypton. This shows how the spiral model defining the neutron core has begun to collapse showing curved instead of linear spiral form but based on the underlying 6-sided fractal form.

One looks at Xe with 77 Neutrons and the backbone model is called into question. In order to understand why this is not a problem, we look to the measured overlapping spirals as set forth in the table above which lays out the math for overlapping spirals which yields for carbon, the six sided structures shown with FIG. 6, being 5.5. When one uses this math result and divides the 77 observed neutron one gets a balanced 14 six sided structures, the 8 six sided structures for Krypton plus the fractally significant 6 more. This shows that the math, as opposed to the visual manifestations, is controlling and continues to provide repeatable, fractal results.

Radon presents a special case. While the neutron count of 135 is a close approximation of the average between 5.5 and 6 (138 Neutron average); Radon also fails to follow the property proton count. However, fractal math suggests a combination. The 4 less protons may be relevant. When the neutron count has 4 removed, it represents a balanced 22 (136−4=132) 6 unit states. The suggestion is that the extra 4 neutrons are balanced by being trapped in the neutron backbone and there remains a balanced 22 six-neutron unit atom with the requisite number of protons (86) to balance it. While hardly dispositive, this matches also with the combined 8 six-neutron unit Krypton plus the 14 5.5-neutron unit Xenon (8+14=22). Unlike the other noble gases, radon is unstable and yields radioactive isotopes if elements including plonium, lead and bismuth, it may be possible to form a stable Radon atom using the modeling to yield the stable/hybrid 22 5.5-6 unit neutron core.

Folding about a core traps information, overlap and information sharing (in chemistry this is paired electrons along fractal spiral overlays) between pair halves explains how bonding works, energy is pretime change.

Folding and unfolding effects may be maximized in terms of folding levels of compression to maximize magnetic, non-magnetic, energy storage, energy generation or other effects related to changing matrices.

As with iron exposed to a shaping field of ct4t11 states, the layout of individual atoms may be mechanically shaped and aligned to hold the desired effect. Any goals of alignment and the means to maximize one or more shapes in matrices lie in the designs and ability to affect them with KFE. Different protons and electrons can be targeted with KFE, although those most exposed to reaction at the ends of atoms have the more exposed electrons and energy applied to center of an atom would tend to flow out spontaneously due to balance to the ends or break the atom up in extreme cases, radioactive decay being an example, where the neutron core is imbalanced enough to be separated.

While more closes associated with electromagnetics, using pretime modeling of electromagnetic signals to be captured and dispersed (as for radar invisibility or energy generation or heat diffusion) is a part of the fractal interaction of CT state exchanges modeled not only about shaping (six sided, for example); but also about the 2\hat{ } and fpix-MI relationships in terms of how the absorbing materials are stacked (e.g. using 2\hat{ }n differentiations in radar absorbing materials as part of structural fractal combinations to increase invisibility or energy absorption in solar as two examples.

Structural changes matching signal shapes of the various ct states can encourage absorption and dispersion or concentration using next higher states.

Broadly, Electrical applications including Magnetic design involves the idea of using lower CT state design for modeling.

One of the major proofs of the model is the ability to describe in detail the process of magnetic attraction and repulsion which, while understood in terms of scale and post atomic effect, was not understood. As with chemical reactions, a better understanding of electro-magnetism will allow for improvements in electronics. Crucial to this are things which are primarily covered in subsequent patents having be developed by applying the modeling to successive processes, including electron pairing resulting in ct4t13 states. Early calculations suggested that electrons were half ct4t13 states (5.4 ct4t12 states by observed weight). The overlapping spiral model when applied shows how electrons paired. Likewise, when the fractal building of the ct4t12 states from ct4t11 states provides information on how electrical transmission works and how ct4t11 states with higher pretime change can be concentrated and used to power electrons or lower their energy. While traditionally this is viewed in terms of amplitude and wavelength, AuT shows this to be pretime locations of particles. From microprocessor to grid designs applying this modeling to those problems will lead to improvements.

### QC-Pretime Transistors

A (pretime transistor) PTT is, essentially, a triode; substituting pretime change features, ct4t12 and lower for electromagnetic output. Like a traditional “vacuum triode,” the control of information into the grid and the resulting readings from drops between the cathode and anode (plate) and control of the heater are all critical to its operation. Pretime locations are used for computing in a post-time environment.

Multiple PTT(s) can be reconciled to determine pretime changes of qubits, and to take these pretime changes and arrange them in such a way as to get interpretive results.

In a matrix, you can get comparison of wavelengths, whether the same or by comparing different length wavelength at the same time. Individual CT states within what post time would be considered wave structures can be analyzed for changes to be used for computing, multiple CT states can be used to compare pretime changes so that the changes pretime can be organized to interpret in a post-time environment.

### Fractal Maximizing Mass/Energy Transitions

FIGS. 8-11 deal with energy matter interface, specifically the exponential interface shown by large scale phenomena. Regardless of the base states involved, at large scales 2\hat{ }n exponential compression is observed.

shows heating based on fractal transitions maximizing interface. The double outer area reflects the more diffused heat; targeting where to have transitions between areas can be maximized. With heat flow biasing it is possible to use one side with a physical or effective valve to allow energy or molecules carrying the energy to move and this can target the cool wall farthest from the heat source and shown as those at the end of the energy transition or the hot wall shown at the beginning of the energy transition or those within the transition space depending on the desired effect.

The exact arrangement of where and when the exchanges occur using AuT fractal design can be varied for different purposes. This requires looking at different base transitions where decompression is based on base number transitions and the applicable exponential transitions. Targeting those at mechanical, molecular or atomic levels allows for different energy transitions to have maximized results.

Part of this process is identifying the “Core locations” possible, those where the central energy is generated which may be identified in reverse by finding the outside wall of one of the exponential transitions and working back using exponential decompression of the defining spheres or circles.

### Explosive Force

The Explosive force can direct it to an edge or a central point at which it may be further dealt with by other reactive armor features, secondary explosions or thicker armor, or free from critical locations in the target. Energy release in whatever form represents an exponential change in dimension.

FIG. 8 shows the 2\hat{ }n targeted compression. N1circle 645 is the inner circle representing the initial area where energy transitions occur, where pretime change is freed where it is shared. This would be the neutron breaking apart in an atom. A less precise example would be the edge of a radioactive element or the center of explosive discharge of ct4t12 and lower states from a chemical reaction. This can be determined by finding the impact edge, here n4circle 646 of the explosive force. In between items 645 and 646 lie the other 2\hat{ }n transitions, n2circle 647 (radius 2 times the radius of the n1circle) and n3circle 648 (radius 2 times the radius of the n2circle). While this is a theoretical model of explosive force, it is visible in NG2775, and the so-called BOAT gamma-ray burst.

Fractal mathematics being what it is, the n2circle is equal to the area between the n3circle and the n4circle as shown by the n2circle1 649 and the n2circle2 650.

This relationship can be targeted in either direction to target forces for reactive armor, explosives, energy capture, energy release or chemical reactions.

To implement this theoretical model of explosive force in practical applications, one would need to first identify the critical locations in the target that need to be protected or destroyed. Then, the explosive force would be directed towards either an edge or a central point of the target, depending on the desired outcome.

If the goal is to protect critical locations, the explosive force would be directed towards an edge of the target, where it can be further dealt with by other reactive armor features, secondary explosions, or thicker armor. This would require precise calculations of the impact edge, as shown by n4circle 646 in FIG. 8.

If the goal is to destroy critical locations, the explosive force would be directed towards a central point of the target, where it can cause maximum damage. This would require identifying the inner circle where energy transitions occur, as represented by n1circle 645 in FIG. 8.

In either case, the energy release from the explosive force would represent an exponential change in dimension, as described in the original statement. The other 2\hat{ }n transitions between the inner and outer circles, as represented by n2circle 647 and n3circle 648 in FIG. 8, would also need to be taken into account for precise targeting.

Fractal mathematics could be used to further refine the targeting of the explosive force, by identifying the relationships between the different circles and targeting them accordingly. This could be applied to a wide range of applications, including reactive armor, explosives, energy capture, energy release, or chemical reactions.

### Armor

Regions can be targeted to get maximum effects both to do damage and to prevent it.

The invention relates to a system of fractal overlays and fills, particularly for use in siding or armor, which provides absorption and collapses in a fractal manner. The system can also be reversed to control explosive features or impact locations between explosives and fractal generating layers.

Targeting exponential change, repeatedly and in either direction, the transition from exponential (e.g. 2\hat{ }n), f(x)-identified as fpix to MI or MI to fpix based change to absorb or repel force, force being the more fpix oriented dimensional feature as shown at centering location 1003 where the desired point of the energy, here the 3^{rd} edge from the center of the energy transition above is targeted here where it matches at the point of connection 1001, a transition as with a corresponding fractal energy release response such as an explosive charge 650 triggering n3circle1 652 which in this case is offset by the overlap equal to the diameter of item 646 to allow it to be controlled by redirection, diffusion, absorption, etc.

The materials used in the system can be varied to produce the same fractal transitional effect based on the fractal differences in strength due to material composition and thickness to mimic fractal effects, such as a water layer which is the same size as a gas layer but with fractally relevant differences (e.g. 2\hat{ }x) in compression.

As has been shown in other figures, the 6 sided “Honeycomb” can be used in connection with circular patterns to combine neutron interactions with fpix (circular) and 2\hat{ }n (circular) effects.

The system of fractal overlays and fills described in this invention can be used in siding or armor to provide absorption and collapse in a fractal manner. This system can also be reversed to control explosive features or impact locations between explosives and fractal generating layers.

To target exponential change, the system uses a transition from exponential (e.g., 2\hat{ }n) to MI or MI to fpix based change to absorb or repel force. Force is the more fpix oriented dimensional feature, which is shown at centering location 1003. The desired point of energy, which is the 3rd edge from the center of the energy transition above, is targeted at the point of connection 1001. This transition corresponds with a fractal energy release response, such as an explosive charge 650 triggering n3circle1 652. This circle is offset by the overlap equal to the diameter of item 646 to allow it to be controlled by redirection, diffusion, absorption, etc.

The materials used in the system can be varied to produce the same fractal transitional effect based on the fractal differences in strength due to material composition and thickness to mimic fractal effects. For example, a water layer can be the same size as a gas layer but with fractally relevant differences (e.g., 2\hat{ }x) in compression.

The system can also use the 6 sided “Honeycomb” in connection with circular patterns to combine neutron interactions with fpix (circular) and 2\hat{ }n (circular) effects. This allows for maximum effects to both do damage and prevent it by targeting specific regions.

The defensive compression can be extended to multiple nested exponential compression states, or the system can be designed with offensive capabilities that increase explosive power within the maximum expansion levels described for the transitions.

The proposed invention, incorporating fractal overlays and fills, can be used in various applications to harness energy and momentum exchanges. This includes the ability to absorb energy from explosives, radiation, and other sources, as well as control explosive forces by manipulating fractal transitions. The system of fractal overlays and fills described in this invention can also be used to absorb the maximum amount of energy for heating water for a boiler and powering a piston of a given fractal size and shape.

Overall, this system provides a new and innovative way to design armor and siding that can absorb and collapse in a fractal manner, while also allowing for precise targeting of explosive force and impact locations.

Overall, the system of fractal overlays and fills described in this invention can be used in a wide range of applications, including absorbing energy for heating water for a boiler and powering a piston of a given fractal size and shape. The fractal transitional effect allows for maximum absorption of energy and transfer to the desired location, making it a highly efficient and innovative technology.

The invention incorporates the use of multiple nested KFE, such as exponential compression states, or “initial scale” spheres, to control the distribution or absorption of energy secondary spheres can continue the transfer. The use of different sphere sizes, overlapping spirals, and a combination of shapes can be used to concentrate or disperse energy, collapse energy into inner circles, add “spring” to the circles, and increase energy transfer. Overlapping spirals, separated by surfaces represented by the line walls which can have varying thickness to increase or decrease the effects.

Different materials, varying the composition, shapes and thickness to achieve the desired fractal transition effect.

The materials used in the system can be varied to produce the same fractal transitional effect based on the fractal differences in strength due to material composition and thickness. The system can also be designed using “honeycomb” and fractal construction, such as six-sided or three-dimensional “Fullerene” modeling in place of circles and spheres, or by using nested circles or half circles. FIG. 8 shows Fullerenes or the proton level (e.g., 14-sided) equivalent can be used in conjunction with other fractal features, as shown above in place of spheres or circles. These are AuT fractals because of underlying MI 6-, 10-, 14- and 18-sided balancing framework at neutron and corresponding stepped fpix balancing at proton levels, for those examples 6, 10 and 14 as shown for carbon and the higher end fractals for silicon modeling.

Modeling allows for explosive exchanges based on frozen in time features as well as these transitions and sub-transitions. These can be organized to defleCT to a center or an edge where smaller charges or exits can make the shielding more effective; targeting smaller elements with the force down to quantum exchanges

Incorporating these in sheets and having fractal designed overlaps and strengthening (such as a six-sided wall within a curved wall or having M diameter circles reinforcing the higher circles as shown above.

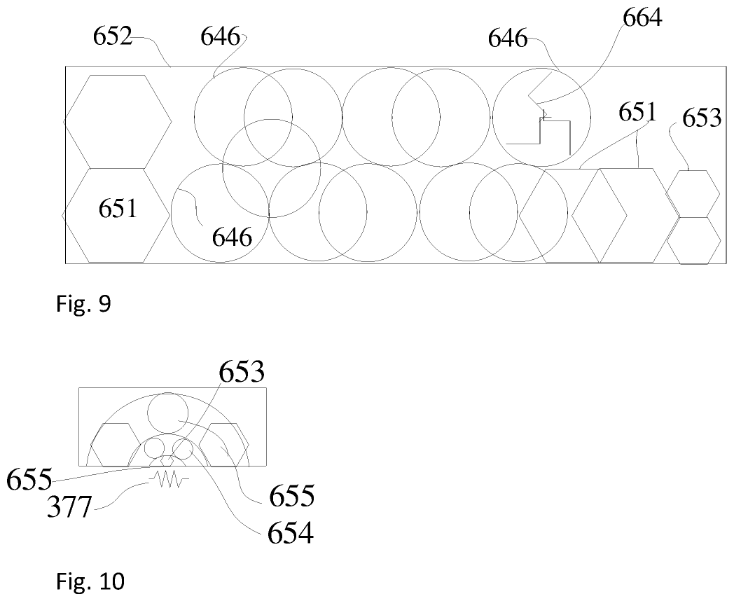

FIG. 9 shows embedding which can be used to maximize Explosive shielding. On the left, fractal Overlays allow the collapse of one into the other but along 2\hat{ }n fractal lines. The openings may contain fillers with fractally relevant fillers to provide absorption nested to have the same fractal collapse features. The exact arrangement can be varied to maximize absorption or release of information using KFE fractal features can be, for example, used as fillers without armor or warheads.

Six-sided or three-dimensional “Fullerene” modeling is shown in FIG. 9 in conjunction with circles or spheres or and with nested half circles to ensures that impacts along relevant lengths are maximized or to take advantage of the fractally relevant shapes which are KFE. For purposes of an example, FIG. 9 shows a sheet of two rows, some of which are populated with hexagonal shells 651, some with circles, here n4circles 646 are shown and some have these overlap to get the benefits of both. As with the circular or spherical structures 646, the hexagons can have fractally relevant fractal smaller hexagons 653 shown here. One additional item is reinforcement using “solid” conversions, here with a bracing spiderweb spirals 664 within one of the n4circles 654, although the number and location can be varied. Here there are three overlapping spirals, the bracing mimicking the other conversion of mass features above the neutron level, the idea being to show options to transfer force using fpix or MI of 2\hat{ }n features according to the modeling and needs for a particular application.

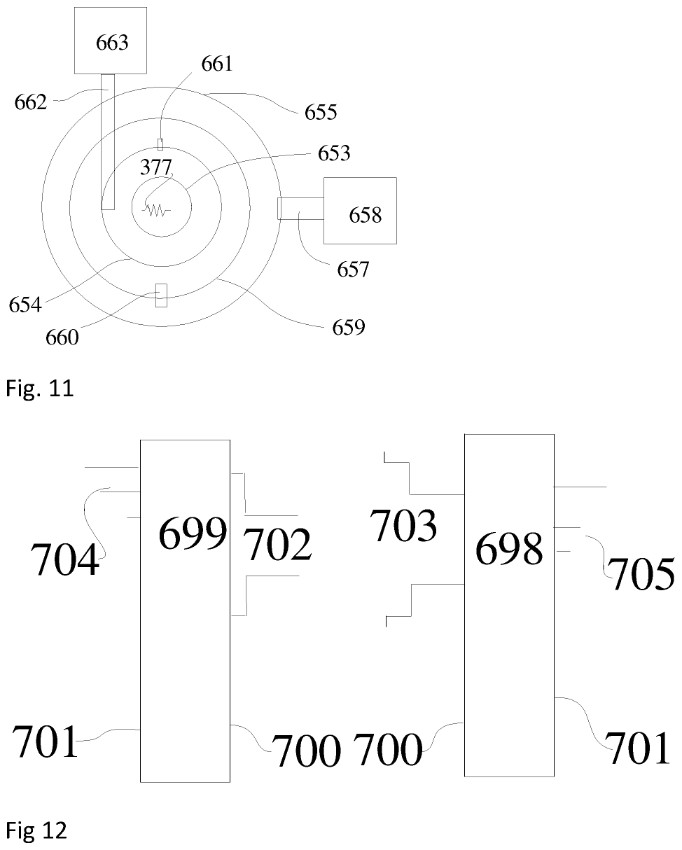

FIGS. 10 and 11 show this design being used in conjunction with a heater means 377 which could be a traditional heat source including radioactive rods. The fractal smaller hexagon 653 in this case corresponds to the 2\hat{ }1 surface area of the heater means 377. The flat surface of the hexagon 655 represents the interface between the heater means 377 and the hexagon 653. The 2\hat{ }2 pipe 654 and

2 4 pipe 655 can be round or hexagonal or both as shown.

To heat water for a boiler, exhaust or other purposes, fractal overlays and fills can be designed to absorb and collapse in a fractal manner to maximize the amount of energy transferred to the water. The transition from exponential (e.g., 2\hat{ }n) to MI or MI to fpix based change can be used to absorb the energy and transfer it to the water. The materials used in the system can be varied to produce the same fractal transitional effect based on the fractal differences in strength due to material composition and thickness. This allows for maximum absorption of energy and transfer to the water.

FIG. 11 shows reservoir 658 which connects to the area between interior wall 659 and 2\hat{ }4 pipe 655 at the n3 circle 646 (not shown as it would duplicate the wall location) by way of valve 657. Interior wall 659 is positions at the halfway point of the area between item 2\hat{ }4 pipe 655 and 2\hat{ }2 pipe 654 (at the n2 not shown as it would duplicate the wall location) circle which is an area of reduced energy based on fractal compression. Continuing inward second valve 660 is fractally smaller (½ the size in this case) in terms of flow to first valve 657 and allows the flow into the area between items 659 and 654. The third valve 661 allows the flow into the final heat exchange area between items 654 and 645. The heat source 377 in this case has a reaction area at n1circle 645 and practical considerations might mean that a wall at n1circle 645 would be impractical (because of the heat). Here there is exit valve 662 which allows the flow of hot water or other liquid to work area 663 which in a boiler would connect back to the reservoir 658.

There is another wall not used at the n4 location which could be added as well as additional equivalents to the interior wall 659.

To power a piston of a given fractal size and shape, the fractal overlays and fills can be designed to absorb and collapse in a fractal manner to maximize the amount of energy transferred to the piston. The transition from exponential (e.g., 2\hat{ }n) to MI or MI to fpix based change can be used to absorb the energy and transfer it to the piston. The materials used in the system can be varied to produce the same fractal transitional effect based on the fractal differences in strength due to material composition and thickness. This allows for maximum absorption of energy and transfer to the piston, which can then be used to power machinery.

The layout of “solid” states (neutron level and greater) and those with more circular applications (protons to energy) for transitions is inherent in this; but also acknowledging where they both apply at the atomic level in different degrees according to whether the proton and neutron counts are the same (1-10) and where they begin to transition as well as those isotopes where transitions to greater or lesser than balanced are emphasized.

On the right, heaving dimensional spiderwebbed spirals, having a flexible hexagonal core instead of overlays but to give a similar elastic effect and also showing a spiral arm replaced with a hexagonal arm (one or more) to show variations.

Different “initial scale” spheres allow targeting different scale transitions used in connection with the resulting 2\hat{ }n, fpix, and MI compression/decompression states or even combinations as with neutron overlapping spirals within 2\hat{ }n spheres to maximize strength; e.g. multiple “overlays offset from one another” or “spiderwebs” of overlapping spirals to concentrate or disperse energy, to collapse energy into the inner “2\hat{ }n circles,” to add “spring” to the circles; to “hold the circles together” to concentrate explosive force or to protect from the inside out.

Round and/or hexagon (could be hexagon with hexagon) alternatives shown with overlapping spirals of different sizes to take advantage of different circle/spherical diameters.

KFE fractally relevant numbers of these nested systems, spacing, overlapping with each other or spirals, the share of the ultimate layer (as in a flat sheet, curved sheet, etc.), half spirals forms and the like are envisioned.

The overlapping spirals are shown as lines, but as shown above they can have any thickness to increase or decrease the effects.

Energy may be tapped at different energy levels and resulting frequencies. If you have a circle with a radius of n, a comparable circle with a radius of n/2 reflects 2\hat{ }(½) exponential compression.

The proposed armor incorporates a unique fractal technique for its defense mechanism. In the event of an outer wall breach, an explosive with a specified range is triggered immediately behind the breached wall. This system allows for multiple outer walls and multiple explosives with varying ranges, resulting in a proportional defense explosion that matches the intensity of the attack.

To maximize the heat capture, the exchange medium may be positioned to provide an interior maximum expansion, which contributes to the overall exterior maximum expansion. Similarly, contractions may be optimized in a complementary fashion. The goal is to achieve sequential expansions and contractions along lines identified by KFE, and to identify where one expansion or contraction is maximized within the desired matrix. The heat absorbed may be monitored, and once the exchange medium reaches its maximum capacity, the next expansion will commence.

For weapons, KFE governs the shape of charge, along with the location and shape of shell casing. Multiple outer spheres of fractally relevant sizes to begin at the maximum force (center or offset from center) of other force exchanges and the effect can be mimicked with explosives with different ranges the defensive explosion ends up matching proportionately the explosion to which it responds in this fashion potentially leaving portions of it on triggered by taking advantage of the AuT defined exponential scales and exchanges.

For defensive charges It may be centered for each maximum expansion So an interior maximum expansion May be part of a net exterior maximum expansion for contractions the opposite process could be done picking up at the middle of another defined area of exchange in this example. The designs can be used to achieve relatively fine transitions in the direction in which force is applied which can help in any reaction where CT state change in used in place of the less precise forces used with explosives which can help with setting up offsets and balance in the very small fusion transition spaces. The ability to get one expansion at the right place to enforce the next is a target as is the ability to get 1 contraction after another for the opposite effect and to use KFE to model where expansion is maximized in terms of a specific exchange of specific CT states.

### Construction

The same designs can be used to get strength for construction materials because static exchanges of information are identified. Reinforcing a series of tubes, including interlocking tubes, using these layouts would allow additional strength. While curvature is widely used, this is using KFE to design embedded tubing targeting the transitions where the maximum force can be targeted for different exchanges.

### Energy-Matter Transitions

This is shown above with nested half circles or pipes, fractal increasing to maximize heat transfer. KFE Fractally modeled overlays and fillers can maximize absorption or reflection or release of different CT states.
1. Unlike other models, this one allows transitions to be considered free of time and of traditional energy concepts, instead focusing on the quantum nature of change and the effects of force reflecting net folding and unfolding along fractal lines between matrices.
2. Creating an effective central generating area, the inner circle of the 2\hat{ }n compression circles, by fractal design of the fission core to match the areas of concentration represented by the compression circles.
3. Absorbing the energy with corresponding fractal absorption techniques and layouts where the most heat can be extracted most efficiently.
4. Apply fractal math to reconcile existing data with common fractal features to enhance its usefulness. Reorganize and potentially reimagine existing modeling and empirical data with fractal plasma algorithms.

This incorporates both old and new views of fusion, fission and chemical reactions considering MI and Fpix observed modeling for subatomic-based phenomena, exponential compression, 2f(n) spiral balancing along with exponential, 2\hat{ }n compression and decompression.

Fractal compression chambers can be designed using nested half circles and spiral conduction to maximize absorption of energy from a thermal source. The holes in the fractal compression chambers can be viewed as pipes seen in cross-section with the bigger pipes gradually getting smaller or the small pipes gradually getting bigger as the propellant or other coolant or heat transfer gas or liquid moves back and forth to maximize energy as pretime change in CT states is transferred using KFE fractal compression and decompression elements.

### Fractal Overlays and Combinations

1. Overlap may be as a fractal ratio such as 5:6/5:12/1:2 and with hexagons instead of circles.
2. Materials can be varied to give the same fractal transitional effect based on fractal differences in strength due to material composition and thickness.
3. This can be used to absorb energy at any level, macro explosives or absorbing radiation.
4. Design fractal overlays: Fractal overlays can be used to collapse one layer into another along 2\hat{ }n fractal lines. The openings in the fractal overlays can contain fillers.
5. fractally relevant fillers that can provide absorption and nesting to have the same fractal collapse features.

### Fractal Overlay and Fill System:

### A. Fractal Transition:

1. Targeting transition from fpix to MI or MI to fpix for absorption or repelling force
2. Force fpix oriented dimensional feature
3. Transition between states
4. Exponential change
   a. F(x)=fpix or F(x)=MI overlapping spirals
5. Capture at concentration points
   a. At circle at edge of force where inner circle touches the explosion/release
   b. Using shapes (hexagon in particular) to focus at the edges.
   c. areas where energy transitions are highest.
   d. Reverse process to get fusion type effects.
   e. Different levels for different types of energy
   f. Determine range of force within areas
   g. Heavier reinforcement at places where walls based on exponential change energy is highest.

### B. Design Variations:

1. Design using “honeycomb” and fractal construction.
2. Six-sided or three-dimensional “Fullerene” modeling (in place of)/(with) circles and spheres
3. nested (half circles)
4. Round combined with hexagon (or hexagon with hexagon) alternatives,
   a. spiderweb of overlapping spirals of different sizes, to take advantage of different circle diameters.

### C. Material Variations:

Materials can be varied to produce the same fractal transitional effect based on fractal differences in strength due to material composition and thickness.

### D. Thickness:

6. Overlapping spirals can have any thickness to increase or decrease the effects.
7. N, N/2: width reflect exponential change.

### III. Applications:

1. Defensive compression can be extended to multiple nested exponential compression states
2. Defensive decompression
3. Offensive capabilities to increase explosive power within the maximum expansion levels described for the transitions

### A. Energy Absorption:

Can be used as energy absorbers to absorb heat, light, or other forms of radiation, including radioactivity.
Absorbed energy can then be transferred or utilized for other purposes, such as heating water to turn turbines B. Fission Reactors:
Absorb heat generated by the fission reactions within the reactor.
Provide shielding for alpha and gamma rays to protect the environment.

### IV. Potential: A. Potential for Energy and Momentum Management

### I. Introduction

Overview of explosive force and its application to reactive armor
Importance of force as 2\hat{ }n compression/decompression (folding/unfolding at FPIX, overlap at
MI) energy release and energy transitions in armor and airplane skins.

### II. Fractal Modeling and Energy Transitions

MI and fpix transitions and their exponential compression and decompression
Incorporation of fractal, 2\hat{ }n, and f(n) times x reinforcing spirals in rocket bodies and other parts
Overlapping spirals and 2\hat{ }n components for controlling and manipulating force
Collapse of solid structural components following overlapping spiral form
Fullerenes b/c of underlying 6-sided framework at neutron and proton levels
amount of pretime change in individual atoms or matrix.
Centrifugal Forces vs straight line and fractal math

### II. Discussion of Explosive Force and Reactive Armor A. Explosive Force and Thermodynamics

Energy release represents an exponential change in dimension.
Collapse of solid structural components following overlapping spiral form
Exponential separation and MI hardness and spiral forms to absorb shock.
Collapse around fractal lines to deflect energy to edges as well as absorb it.
Maximize energy and momentum exchange at inflection points, fractal circles to increase fusion bomb and fission trigger.
Distribute, redirect, absorb, push and disperse energy.

### Fractal Overlays

Explanation of how fractal overlays allow for collapse along 2\hat{ }n fractal lines.
fillers for siding or armor, along with fractally relevant fillers for absorption
arrangement of fractal overlays can be varied to maximize absorption or release of information. strength and transitional effects and why they are the same.

### III. Spiderwebbed Spirals

Modeling suggests that spiderwebbed spirals can be used for energy dispersal along with a flexible hexagonal or circular core. Spirals are the underlying transition, circles form from the fpix spiral math just as MI elements form from the MI spiral math. The amount of overlap in fpix is unclear although the suggested modeling is shown in FIG. 1. This shows how the fractally relevant circles generated match the offset MI spiral forms which are expected at higher compression when the fpix shifts to MI. The relationship of exponential circles or spheres of force is matched, and atomic structures are mimicked at post atomic scales whether observed based on Fpix or Mi.

Since circles can be replaced with, for example, hexagons, spiral arms can be replaced with arms made up of hexagons or circles for effect.

Multiple nested exponential compression of different sizes impact different transitions which can be modeled fractally with this modeling.

This modeling can be used to control electronic component moves, not just electrons, but also allowing the charge components of AuT positrons to migrate.

### IV. Energy Absorption and Utilization

The forms can be used for energy exchange as with ball bearings carrying energy away at different locations, the degree being maximized by using this modeling.

### Electromagnetism and Repulsion and Using it to Target Fusion:

Force in the broadest terms is change in the quantum count. At the level where it becomes useful in its various forms as different forces can be viewed as pretime change. Electricity and magnetism are the net directional movement of ct4t11, t12 and t13 states.

The ct4t11 magnetic portion rolls the ct4t12 electric portion, giving pretime change and direction to the lower pretime change ct4t12 elements. The smaller “electric current” ellipse is where the positrons are broken with excess t12 states into 5-unit ct4t12 electrons (e−). This ellipse is not as visible, but it is a fractal equivalent of the observed magnetic elliptical “field” which is shown to be a collection of t12 states broken into M+5-unit ct4t11 state magnetic features.

Spinning a magnet to get electricity or spinning electricity to get a magnet is well known, targeting the movement of the ct4t11 and 12 states is not known as the concept of using these could not have predated knowing they exist and their relative interaction as the less compressed states coming off and coming back to the more compressed as absorption and spew.

One can see how this “field” can energize a free wire that is put in a position to pull absorb a portion of the M+ states (as well as the t12 states) which disrupt the t13 states (in the wire this would largely be positrons sticking out from proton cores and electrons) energizing the resulting free t12 and e-states so they move down the wire.

Based on this modeling, the more you can concentrate magnetic fields on electrical flow at the right location, where the maximum ct4t11 states and particularly the maximum M+ are pushed onto the conductors, the more energy extractable at the location. Likewise, the more e− that is present in the wire, the more effective the M+ mix is.

The effect of a wire pushed into a stream of magnetic or electric effects. The wire has it's t12 states (ct4t12) pushed along and energized by the t11 states and composite M+ states (3-5 T11 states) circulating within the field.

The electric part of this is almost certainly primary and effectively perpendicular to the magnetic portion.

Repulsion creating “space” and attraction absorbing “space” in the form of the pretime and therefore invisible CT states revolving (and in pretime generating wave and magnetic features) about the electrons.

One view is that space is created by unfolding. It can also be created by lower CT states being concentrated between higher (post-space, ct4t11 and higher) CT states, as way “energy” yields work.

We can use this modeling to deflect radar and absorb radar (or other energy), to do computing by moving pretime change around and to reduce the amount of intervening information (space) or create more compression to bring together neutrons for fusion reactions or separate them for fission as well as at higher scale for chemistry.

Another view is that this building of space is reflected by having “uncombined” offsetting pressures, the fractal equivalent to atmospheric phenomena and the High and Low cells can have the concentrations necessary to have the effects shown created by the dispersal and recompression of the fields in the competing magnets.

Repulsive magnetic fields are when the buildup of free M+ increase relative to T12 states and attractive is when there is a buildup of T12 states relative to the M+/T12 mix. These T12 and T11 states have relatively and on average high pretime change and hence give movement to the e−. On the left you have the collapse of M+(5 units of t11) collapsing from both ends into ct4t12. Repulsion doesn't create space, but it changes how much room it takes up.

### Electromagnetic Dimensional Offset

The post time elliptical occupation of M+(the theoretical 5ct4t11 magnetic equivalent of the 5 ct4t12 electron) is shown relative to the electron elliptical area and this pretime movement is what gives it the energy and force we use, the M+ being less dimension occupies more places than the electron at any point in time but is exponentially smaller as shown.

Look at how changing magnetic field represents moving M+ states against e−, especially moving a nonmagnetic copper plate through the middle of a horseshoe magnet (p on one side of plate, n on the other).

This “field” can energize a free wire that is put in a position to pull absorb a portion of the M+states (as well as the t12 states) which disrupt the t13 states (in the wire this would largely be positrons sticking out from proton cores and electrons) energizing the resulting free t12 and e− states so they move down the wire. Presumably from this drawing, they do not move back readily because of the larger t12 states entering the wire from the other end.

As can be seen if information is injected from both sides, there is repulsion, but if it is injected in one side and folded in the other there is attraction. The other function of repulsion is to unfold even exponent states into odd exponent states. Any way that the net space is increased will result in repulsion as the energy of freed lower CT states expands the matrix.

### Claimed Uses

Manipulation includes using changes in pretime (pre ct4t11) CT states for determining pretime change for at least one CT state and basing communication or computing results on those pretime changes. By knowing the amount of pretime change on both ends in the information provided, communications can be made in a time free environment and the smaller the informational CT state accessed, the less time dependent it is although the overall amount of information decreases. Using lower pretime states means that more information can be sent, for example, if information is sent in ct4t11 states within a single ct4t12 states there are 10 bits of ct4t11 for each one of the ct4t12. If electrons are used, there are 5 times 10 ct4t11 states for each electron (approximately) in a pair so that there are 50 states doing the same computing and/or communications as the single electron.

The process of isolating Grid components, breaking them into their fractal components, and showing how these operate and fail within the larger matrix of the grid. This can be expanded to apply fractal elements to improve the grid and the components. The matrix can be a device (cell phone, generator); a self-contained unit (boat, ship, house), a city or a planet, fractal mathematics embodied in AuT applies from pre-field elements through the entire grid and the grid's environment.

Following AuT design with KFE using spiral layout, relative size (2\hat{ }n, spiral or both); separation of submatrix within the battery matrix (shown here with submatrices of (1) the cathode, (2) the anode, (3) an intervening layer between the two, and the other methods suggested by KFE can all be used. Spiral changes inward or outward towards the anode or cathode along preferably spiral pathways like spiral 214 towards the anode. Stepped charges at different locations is envisioned toward or away from area where buildup of ions is desired using ct4t11 states based on the amount of pretime change, size of delivery (branch or main line with or without breaks to spread out waves) which work to dissolve and solidify depending on the target, the outer electrons of Lithium shown by the number, size and spiral layouts of the carbon antennae including a dispersal array used to dissolved those atoms which would otherwise be forming dendrites.

Spiral charging and discharging (movement of the location where ct4t11 are put in or changing the movement of ct4t11 once it is inserted) can also be used to keep electrolytes circulating, disperse and limit dendrite formation or encourage dendrite dissolution.

Balancing can be used on either side and on top and bottom of cathode/anode.

FIGS. 3 and 4 show how the interchange of energizing CT states can be used to move other CT states since the circulations and exchanges and impacts are dynamic.

Battery-move concentrations of pretime change/release pretime change as focus. The designs can push charging ct4t 11-13 states to concentrations just like piling water into a dam. It can also push discharge to break down dendrites, for example. As the ion size changes and the liquidity change, the features modified and how they are modified changes, and with our modeling we can change the nature of the electrolyte as ions or charge move through the separator-carburetor so in this case, electricity would have a different pathway (wire, for example) than the OH radical which might require a more fluid environment.

NI—Fe example:

Specific objectives are to target the suspension (prevent discharge), use and movement of OH-(hydroxyl radicals) (increase specific energy) in a Ni—Fe battery to increase efficiency.

Discharge: All batteries are fractal equivalents of capacitors but at different CT levels (electrons are 5ct4t12 states, protons and neutrons are 10ct4t15 states). By applying fractal mathematics, the capacitance (stable charge) of charged Nickle cathodes can be increased. In the casing shown, negative information exchange is used to stabilize charged (OH− ion) Nickle. At least one of two sides of capacitor is embedded in the casing walls or circulatory openings in the carburetor which are active except when the battery is discharging to reduce standing discharge.

Specific energy: Energy release from a system is the release of low CT states with high amounts of pretime change, particularly at the ct4t11-ct413 level. Iron oxidation is the equivalent of carbon oxidation. Therefore, a properly operated carburetor can maximize the efficiency of the reaction, in theory, between the Fe fuel and hydroxyl radical oxidizer. The focus is on interaction between iron and OH− radicals beginning at large scales but targeting ctet12 release. The capacitors have a double function of staging the transition when sufficient information (charge) is built up to maximize the efficiency of the reaction.

These atomic structures and manifestations of electrical and magnetic interactions are discussed in more detail in the slide show and more information can be found in the Neutron Periodic Table of the Elements published by the PI. The half-cell reaction at the positive plate from black Nickel(Ill) oxide-hydroxide NiO(OH) to green Nickel(II) hydroxide Ni(OH)2:Fe=26; Ni=28: 2 NiO(OH)+2 H2O+2 e−↔2 Ni(OH)2+2 OH−. The emphasis is on the Neutron backbone and restructured proton core and at the negative plate: Fe+2 OH−↔Fe(OH)2+2 e− (Discharging is read left to right, charging is from right to left.)[17]. Together, AuT modeling shows the simplified cycle of the battery for 1) maximizing OH− radical concentrations using capacitance, 2) improving OH− radical flow using charged valves and 3) maximizing structural transitions between Anodes and Cathodes using fractal bonding and charge concepts at the atomic/molecular level particularly focused on how the OH− radicals are moved and contact the Fe Anode. This involves maximizing the chemistry and “carriers” for the charge, both liquid phosphorous/Lithium carriers and Organic semi-solid carriers and doped semiconductor solid-state carriers.

Iron oxides are trapped adjacent to the anode and the atoms left over circulate back to the cathode cavity for recycling later. The locations foils and charging wires in the carburetor provide breaks in linearity so that the current can move along carburetor wall sections to pull or push OH radicals.

Li ions migrate to either side of a metal plate (Li), and are small, highly charged atoms, and have features when dissolved (AuT modeling) that mimic Helium which makes Li easier to guide. These features can be maximized for negative ions (OH−) for the same effect, here by creating a moderate positive charge in a thinly insulated or aluminum type (tightly oxidized) metal plate used to attract OH− radicals which are then removed with separately charged separator in proximity to the Fe anode; and using Tesla one way valves to bias flow towards iron plates and retrieve uncharged flow or OH− radicals on recharging as the process is reversed.

It is possible to push the magnetic center together or pull it apart, then freeze it in place by removing change along fractal lines (with a hardening electrolyte-effective or actual) then unfreeze it to allow it to fall back into place.

### Ion Batteries Modification of the Film to Incorporate New Atomic Modeling.

Steering for resins including solvents and electrolytes between battery elements; or ions or other states can be done with electromagnetism decompressive when the CT state combinations “create space.” This application is in part this new definition of electromagnetic repulsion, reflecting anti-alignment, vs absorbing space when the “fields” (accurately here the CT states) are opposite, unaligned CT states. When matched correctly the CT states pair and are compressive; but if matched incorrectly as they move closer more space formed from the combinations of CT states.

Modeling argues for t12 and t11 as a twostep process, repulsion at one stage where to states decompress when together, and compression at a second level at the positron. They combine to release “dimensional space.”

With AuT a battery designer has all the fractal elements below ct4t12 to work with, along with definitions of energy and atomic structure fractal design. Features which are at best poorly understood in terms of fields have AuT particle counterparts with pretime change. When concepts like magnetism and fields are considered with this additional detail, magnetism takes on all of the features of waves although one fractal step less compressed and can be used to affect even lower states, just as electricity can be used to manipulate magnetism. Broadly, the process is using AuT features to design foil atomics to increase t12 fluid and ion concentration particularly along lines where it is to be moved as battery ion accelerators. AuT processes target t12 fluid, electrolytes, the typical solvents or semi-solvents between components and semiconductors, particularly at the interface between the anode or cathode and the electrolyte, semiconductor or other “t12 stabilizer” defined as a matrix which can stabilize the exchange of t12 states at the surface, in this case the lithium film surface; changing amounts of relative current, changing the direction of the electromagnetic push or pull involved with opposing magnets; changing the angle relative to the position of the ion film, changing the makeup or spacing of layers of material (cathode, anode, separator, semiconductor, electrolyte) including the thickness of those to vary the ions and t12 fluid to be manipulated; concentrating magnetic effects of information with curves, the thickness and separation of coils or the number of coils.

AuT suggests that opposing magnets generate space with repulsion and there is attraction increase juxtaposed to the point of repulsion as information is pulled in to replace the information going out.

Design includes the number, type and layout, of loops/curves of conductors generating “cooperating” magnet lines of force adjacent to anodes or anode films or cathodes or cathode films.

Relative charges of the conductors; for example, gradual change towards the anode or cathode, edge or center, top or bottom of corrugated foils;

Changing relative charges relative to the ion path, especially the path defined by a charged anode foil towards an anode or cathode foil to a cathode; including focusing the movement of the ions near the surfaces of the foil.

Alignment of fields (angles, surface changes (hollow, solid, alternating the metallic qualities along wires or with alloys of a single material wires to alternate fields on target ions which may form ionic rivers defined by atomic or electromagnetic features.

Timing of charges between magnetic and electric power of the different elements, particularly the conductors and the anode foil and the relative location of the conductors to alternate fields to target ionic paths, including forming “rivers” defined by the electromagnetic features of the ions and the t12 carrier streams which may be concentrated in hollow interiors which may be formed in the wires or may be by corrugating the films to encourage the collection of mutually repulsive t12 states (in this case Li ions) at bottoms and/or tops of the two sides of the film at different locations along the corrugation length, height or width as by varying the atomic structure as by varying the copper concentrations in lithium foils;

Switches, controlled by capacitors or otherwise, to turn current on and off and to change the shape of the coils or magnetic features.

Insertion and organization of AuT antennae to absorb waves of energy or magnetic fields and to disrupt electromagnetic currents at different places, whether in the lithium film (lithium foil) or in the separators or other layers within the matrix of solvents (or semiconductors) between the primary anode and cathode components.

Existing batteries with bracketed top (tm) and bottom (bm) electro-magnets in pairs (p1, p2, p3 for example) on either side of lithium film chargeable separately so that the top of one pair may interact with the bottom of another pair in creating fields.

Aligned exterior magnets moving to center different areas and folds (layers) of folded lithium film to increase potential. Copper-Lithium films a to encourage flow at locations to get the ions at a point where they can be manipulated. The charging of particular magnetic elements can be shifted so that the fields are created not just with opposite magnets, but with magnets that are offset, and the order of charging and neutralizing magnets can be varied to get desired effects including fast charging and potential dendrite dissolution or dispersal. Corrugations may be offset to improve alignment or timing of the electromagnetic effects resulting.

KFE features can be used to improve battery design. The idea here is that the primary elements of Li battery charging are dissolution of electrons to create a Li-ion which is more easily moved, and efficient stacking of the LI-Ion at the “negatively charged” ANODE during charging by non-random deposition which can both be accomplished with streams of ct4t11 inserted at strengths and locations determined to be fractally relevant by AuT Antennae strategically placed for this purpose within the battery matrix.

Charging is done with direct current, one concept is to provide current through wires 585 on either side of the AuT antennae biased with KFE to supply primarily ct4t11 drawn into the solution (not shown) of the battery through carbon antennae 578 opposite ion wires 587, doped with potassium, for example, within the semiconductor matrix 586 of the battery to pull the ct4t11 states through the carbon antennae.

An AC circuit could be used to pull in ct4t11-12 states sequentially into different areas of the battery matrix to maintain the dissolution and encourage the desired circulation of the Lithium ions.

LI-Ion batteries: using fractal structures in wires and lithium to define preferred migration paths.

Battery: target the oxidation and reduction of iron batteries using the fractal steps of the iron-oxygen-anode-cathode-chemical and liquid that allows the transition as well as coatings. Shifting the interface between fractal numbering (e.g., 3 and 6) or from overlapping six sided to aligned six sided to control the oxidation and reduction.

Graphite separation (intercalation) buffers whether by dimensional or feature (fpix base numbering, MI base numbering, particularly in connection with concentration, thickness, separation, shape, etc.) are the unbalanced squares of silicone and the hexagon of carbon builds to increase efficiency. Another way is to introduce 3 dimensional or 2-dimensional exchange places, the 3 dimensional being more accommodating to neutron interactions, the 2 dimensional being more conducive to two dimensional exchanges since the number of dimensions increase between neutrons and protons. Targeting this relationship with chemical structure or with physical structures allows the biasing of interactions along with the other fractal features.

Spinning of magnets adds pretime change in the form of movement to the ct4t11 states which revolve around the iron electrons of the magnet which can be transferred through contact with similar states impacted with the field and these impacts may be targeted using the methods indicated to maximize the exchange.

Based on this modeling, the more you can concentrate magnetic fields on electrical flow at the right location, where the maximum ct4t11 states and particularly the maximum M+ are pushed onto the conductors, the more energy extractable at the location.

The more free or fractally aligned to match the M+ or e− that is present in the wire, the more effective the M+ mix is.

The features at work here are unfolding, unalignment and folding and alignment which are all definable generally as compression and decompression. In the magnetic repulsion model, for example, two halves of 5 M+(possibly 3m+ because of the transition between ct3 and ct4) can be aligned so they combine forming a m+ pair or they can be unaligned in which case there are more of them and the push the magnets apart and push electrons to the extent they can be made to move and supply motive force to the extent their pretime change is transferred to movable electrons (otherwise possibly as heat vibration) and where they bond, it is just like electron bonding where they come together and hold things together where they were otherwise repulsive. This is a very different model of electrons is important that bound electrons provide balancing of proton positrons on either side.

### Details or Batteries and Electric Component Carburetors

### Electro-Mechanical Case/Separator

1) Flow using one-way valve means (tesla type valves are shows) with or without chemical enhanced or electromagnetic pathways.
2) generation of OH within the valve means so the OH flows to the iron and then flows out.
3) bias flows with charge instead of barriers which can be used to reverse direction of barriers using charge between two.
4) Capacitance to stabilize charge.
5) Shaped surfaces to maximize elliptical forces.

Capacitors: looking at this as a large capacitor it is a system where efficient exchange of low CT states as abs. and spew occur between charge and discharge and goal is to redesign it this way to deal with one of the two problems

The other electro-chemical features of the battery remain largely the same. The shape and ordering of the casing and racks is subject to some alteration. Likewise, while the layout of the one-way valves is shown as alternating, this may be staked or otherwise as the precise effects are modeled.

Figure: see Flat Carburetor/capacitor design one version of separator with AuT Tesla Valves above.

PASSAGES/VALVES: The separator has passages through it for fluid movement,

Tesla Valves: done at separator and perpendicular or angled because that is easy area to work from, might also be at edges or inside of spaces.

Separator honeycombed with openings allowing the flow of electrolyte from one side to the other. This may be done on a surface, or it may be used in honeycombed (as the openings) between plates.
between polls may be coils which do push/pull action in direction sought
Limit distance, maximize flow in range of size and charge of oh−
METALIC/SEMICONDUCTOR control: thin metal plating and wires.

In the separator there are also passages for wiring and a flow system that would be familiar to anyone with a background in unit operations. Again, I have a picture of this, done with a cad program, but not the kind of drawing needed to make something.

ROUNDED VERSION: The plates are rounded to allow the OH radicals to be channeled for subsequent release in the presence of iron and during the charging phase the process of capture and release is repeated with the transition from side to side reversed. Concentric arc (semi-circular) designs combine features of both.

Function: Concentrating or dispersing pattern (laid out or as it operates) especially overlapping spirals or exponential from outside in or inside out depending on operation.

In the case of discharge, the movement and reaction are driven by oxidation reaction at the iron anode and while charging it is driven in reverse by oxidation at the Ni cathode.

Movable alignment with stored magnetism and electricity. Arranging metal alignment . . . pushing apart or pulling together. Imprinting charge difference across the metals. Focus on the electron and atomic line up. Moving edges, may be at a level of Li 3 vs 4 protons. The need is to force it into movement. This can be AC movement or it can lock CT states in place around anode/cathode; opening just the positron so that it pulls electrons to it, may be with perpendicular fields-align unaligned magnetically ions have to move to a balanced layout, how to get an unbalanced regional layout with other freed dissolved metals.

Because Ni—Fe batteries spontaneously discharge, the discharge can be used to plug the openings with negative potential after a charge and before use. In this case, the plates of Ni and Fe or other metal or alloy plates can be held as close to the honeycombed separators as possible to allow circulation of the OH− or other ion or ion groups necessary to expedite the transitions of electrons as the OH− atoms bond on one side or the other.

(Cold welding) positive charged ions and moving electrons; oxygen puts a protective oxide layer to keep them from joining, when oxide is worn away the electrons flow from one to the other.

Energy always flows from concentration to dispersion, except when they don't. This latter phrase is the truth about compression and the problem with thermodynamic interactions below the scale of mass and a primary target of processes under AuT.

AuT modeling can design a pathway for exchange of OH radicals if liquids are eliminated or as here limited in area as with a non-reactive but nevertheless conductive metal.

Instead of dealing with transporting something bulky, AuT allows the larger CT states to be left it at one place and transferring its important features to another which is done with AC circuits accidentally and without the efficiency of using KFE in the process. KFE Pathways and biasing, biasing especially for this one thing is the method used. It is possible to keep large CT states centered and allow backwash of uncharged elements, outside of that pathway in these two ways or reversible systems, you must move something to carry an information releasing and energy capturing aspect both ways. Entropy in this closed system moves in both directions even while external to the closed system or matrix, entropy nets in one direction. movement or pretime change is introduced or released by combinations which trap it or release it and the keys to this are reversibility, ease of capture, ease of release along with those inherent in the purpose, which is the ability to capture and use the release, the speed of capture, etc.

Iron atoms possibly at bottom of corrugated foil could provide half of magnetic pair charged by current in the foil or layers of electrolytes or other semiconductor materials aligned using KFE chemistry and positioning and density.

This can be targeted for different effects, such as ion movement over small areas or parts, especially AuT positron charge movement, transitioning between ion size, externalizing or internalizing carrier ions and their component CT states. Reversable elements like iron-ni interaction yield bonding energy which can be modeled using AuT and the same is true of oxygen iron bonding, the released or captured ct4t12 and therefore activating ct4t11 states make this happen. Pre AuT science basically stops at electricity and magnetism, AuT looks past magnetism to tap into the underlying source of work, quantum change and fuse length.

“Non-existent collision vs actual information exchange is where we find answers at subatomic scales matching those which are recognized, electron exchange, to make batteries function, so that we can make better information exchange and in this example the iron-oxygen to iron Nickle exchange necessary to store information, release it and send it to where it can be used.

With batteries this allows designs to store the energy at a higher level than Li which in AuT can be done with larger metals. In fact, this is regularly done with thermocouples and in space this same effect can be used to fuse metals together. To understand how this would be undertaken requires a better understanding of AuT and its effects on EM.

The ability to build, destroy or push apart barriers and isolate based on concentrations of pretime change means that energy release can be targeted and places where energy bleeds away can be minimized.

Iron “pellets” which are in molecules or allows which allow them to be expanded or shrunk. Shrink metals and push/pull them into semiconductor pockets to be held and then pull them out so they can expand. Use flat and perpendicular layers/fins to accomplish the movement.

Freeze them into salt grids when compressed, dissolve them to let them out.

Electro chemical energy may be held with solidifying electrolyte. HP can be decomposed to hydroxyl radicals through UV, transition metal, carbon-catalysts,

### EM and Solar Shaped Interactions

The shape of leaves can mimic circuits which can be used to maximize solar interactions. While the hexagon shaped structures holding chlorophyll can be seen as a nod to the underlying chemical structure, the ellipse can be seen as a nod to the interaction of electromagnetic forces.

Leaf designs reflect elliptical field interactions which can be targeted using AuT to mimic and improve these interactive elements.

While other biological imperatives likely have a part to play and while the framework of chlorophyll appears to be a nod to the underlying 6 sided fractal structure of carbon and/or carbon building blocks, many leaves have an elliptical shape which could be to mimic this energy layout for better absorption and targeting the KFE for energy transfers is one element important to all electrical interactions and this would include a focus on the draw from (often antennae like) points at the end of these leaves down toward a common stem.

KFE can be used to maximize effects such as alternating wires directionally and as electron donors or receivers (zinc/copper for example) within magnetic field to assist in drawing off or channeling information. In addition to absorbing energy producing fields, this can be used to absorb and disperse information to create radar invisibility.

The lower the compression state the more two dimensional the result so that the t11 states are moving in a more confined, more two-dimensional river. Shaping includes electrically transitioning (the observed spiraling of wires in transformers) or mechanically moving from this two-dimensional stream into the more 3 dimensional and compressively offset (90 degrees typically envisioned) allows for the type of information exchange needed to derive t12 level energy from magnetic fields.

Using the “leaf” form to capture and transformer type spiraling together can enhance the efficiency of the transitions.

This is using KFE based pretime change in specific CT states as to use, store energy or release it, using batteries, solar or different CT state effects (typically electricity and magnetism) as different features of the same pretime change effect.

### Molecular Pumps

This positron coming off an otherwise collapsed neutron is the reason that the absorption of the proton (the source of positive charge) is the same as the spew (unfolding) of the electron which is otherwise 1/1800th the size of the entire proton.

This positron provides a unique qubit because it is positively charged and locked in place on the proton. Focus on securing the place of the electron in qubit applications is not as significant where focus is on the positron which is necessarily fixed to a much more easily managed proton, and which moves already has secured ionic forms of Li in Li-ion batteries.

This relationship is easy to manipulate, relatively speaking, as electromagnetic:

### Fusion

Fusion can be defined by removing non-compressive information to get fusion, fractal-based alignment and balance. Overlapping Fibonacci spirals can be utilized to achieve fractal balance and alignment, leading to improved performance and greater stability in plasma systems. Additionally, the models can be applied to the development of state stabilization techniques, which rely on the transition between exponential transitions, fpix transitions, MI and overlapping MI spiral compressions and the transitions between them. This understanding can enable the development of innovative plasma technologies that are more precise, efficient, and stable, with broad applications in fields such as electronics and materials science.

Fractal modeling looks at balance, fractal structure and, where the states are properly defined in terms of net CT state fuse and positive or negative result, tendencies toward compression as a low-cost alternative to past fusion models which focuses on forcing neutron heavy, impossibly rare isotopes. Hydrogen and using the B-F-C method, get neutrons and balanced atoms. We get to neutrons by either 1) compression A-positrons and electrons or 2) substituting an unbroken t15 state for a broken one or otherwise targeting fractal proton-neutron interactions.

The stream of ct4t11 states is also filled with ct4t10 states and combinations of those. These can combine into low- and high-pressure system equivalents as shown below to create the repulsive effects and attractive effects of electromagnetism. This can be used to create “vacuum” to the extent it changes the concentration of the affected ct4t11 states. The high- and low-pressure system modeling can be used on the center between neutrons and outside of a neutron pair matrix to steer fusion alignment along with electromagnetics in the alternate base modeling to steer the protons and electrons for Fusion.

### Large Atom Fusion

Fusion of larger, less stable atoms, boron being an example, into a more stable form, neon in this case would have benefits since more CT states can be targeted and the need to use rare fuels or to collapse Hydrogen atoms is eliminated. The process steps are the same, except that portion tied to the collapse of hydrogen is eliminated. The goal is that the neutron backbone of the larger atom, here boron, would need to be separated from the stabilizing proton core, the core being dissolved using plasma, a solution of shared information states between the core and the backbone, the two backbones joined by eliminating space between them and balanced about the very small amount of shared information between neutrons, and then the resulting 10 unit backbone stabilized by a new core which, in turn is stabilized with electrons.

Targeting bigger atoms with more stable forms includes looking at Boron which is an less stable form of Carbon with the same backbone or Fluorine which is the unstable backbone equivalent of Neon or aluminum as the unstable equivalent of Silicon and others like Potassium vs. Argon where the proton irregularities (at least as compared with lighter elements) can be targeted more easily allowing neutrons to be added and ending up with more balanced spirals.

Absorption and spew, engines (areas of increased absorption or spew), and shared information fulcrums all would be targeted using the same techniques that are set out for smaller atoms. The electron and the proton cores are cages. To have fusion, you must build a new cage or expand or penetrate the proton cage to insert neutrons within the cage. You must get the neutrons close enough to share information, then get the proton core close enough to stabilize the neutron backbone formed by the neutrons sharing information.

The equipment that could be utilized for each step:

### 1. Neutron Backbone Separation:

Plasma Generator: Employ a specialized plasma generator capable of producing a controlled plasma environment. This generator should allow for precise manipulation of plasma properties, such as density, temperature, and electromagnetic fields.

Containment Chamber: Provide a chamber where the larger atoms (e.g., boron) can be introduced and exposed to the plasma environment. The chamber should ensure a stable and controlled environment for the separation process.

Plasma Manipulation Systems: Use electromagnetic fields, high-energy particles, or laser-induced plasma interactions to selectively dissolve the proton core of the larger atom while preserving the neutron backbone.

### 2. Backbone Joining:

Plasma Control Systems: Employ advanced plasma control systems capable of precisely manipulating the plasma environment. These systems should enable the alignment and merging of the separated neutron backbones.

Plasma Stability Monitoring: Utilize sensors and monitoring devices to observe the plasma characteristics, ensuring optimal conditions for the joining process.

Magnetic Confinement: Apply magnetic fields to confine and guide the plasma to facilitate the joining of the neutron backbones.

### 3. Balance and Stability:

Plasma Adjustment Systems: Utilize plasma adjustment systems to fine-tune the plasma conditions, including density, temperature, and electromagnetic fields. These adjustments aim to achieve the desired balance around the shared information between neutrons in the joined backbone.

Feedback Control Mechanisms: Implement feedback control mechanisms based on real-time monitoring of the plasma properties to maintain stability and optimize the balance of the backbone structure.

### 4. Core Stabilization:

Core Introduction Apparatus: Develop a specialized apparatus for introducing a new stabilizing core structure into the joined backbone. This apparatus should allow for controlled deposition or injection of suitable atomic components to form the stabilizing core.

Precursor Material Handling: Provide equipment capable of handling and processing precursor materials required for the core stabilization. This may include advanced deposition techniques, such as molecular beam epitaxy or chemical vapor deposition.

### 5. Electron Stabilization:

Electron Injection System: Design an electron injection system capable of introducing electrons into the stabilized core structure. This system should enable controlled electron configurations to match the requirements of the core and promote stability.

Electron Beam or Source: Utilize an electron beam generator or electron source to produce and direct the electrons into the core structure.

Throughout the process, advanced monitoring and diagnostic systems should be implemented to gather real-time data on plasma properties, stability, and fusion reactions. High-performance computing systems can aid in simulation, data analysis, and optimization of the fusion process.

It's important to note that the above equipment description is a conceptual outline, and the specific design and implementation details would require further research, development, and engineering based on the scale and practicality of the fusion system.

Fusion is the transition from ct4 transitional states to ct4 states (neutrons) and early ct5 transitional states (atoms) with the release of lower information states having more pretime change which at certain levels is viewed as energy.

Applying the better proton model, neutron backbone, balance of absorption and spew and the resulting structures and stepwise building are inventive steps towards fusion and understanding the mysteries of the proton-neutron interaction.

The model shows that the atom has a neutron backbone supported by a proton core which is supported by electrons. In a sentence, existing reactors all attempt to hold plasma within a magnetic containment vessel, AuT shows this method to be contraindicated. In other words, no one is trying to do fusion the right way.

### Fusion Focusing on Hydrogen and CT States

Given the modeling, fusion can be targeted at any fractal transition beginning with the proton-neutron transition along with dual fractal (fpix and MI in the case of ct4 fusion) balancing. The application of forces (changes in CT state matrix) should transition from targeting arrangements using fpix when dealing with the proton elements to MI based arrangements when dealing with neutrons.

One step is using Plasma and/or M+ to separate the 10-ct4t15 so a complete t15 may be substituted for a broken one with a positron or collapse the positron-electron pair so that a neutron can form in a balancing matrix of at least two neutrons, balancing proton cores and electrons to balance the neutrons and possibly providing intermediate balanced states while the neutrons are brought together.

One alternative is a flow of plasma being impacted or impacting a stream of hydrogen in the 5; 3; 2; 1 or 5, 3, 1 pattern in terms of concentration or compression or removal or adding of information in the form of different CT states to get a continuous fusion at the point of hydrogen entry instead of focusing on the plasma generation.

The use of magnetics and winding/unwinding creation of space allows targeting the creation or elimination of space to guide or this combination. It is desirable to consider designing at least one wall of chamber as a focusing lens magnet or part of a magnet within magnet pair (inverter magnet) with this lens magnet being adjacent to the reactants. This could occur with multiple walls so that it is stage as one half of inverter magnet or focusing feature targeting fractal features seen in a finished neutron and neutron pair or to fuse neutrons on either side of a larger atom which is balanced or unbalanced in a way to encourage this bonding with the release of pretime information or absorption of it depending on the desired effect.

A good way to focus energy is to use a reactor chamber shaped to mimic the end result desire at a higher scale, including having virtual layers within it as one of the magnets or half of inverter magnets, designing the shape, magnetic and nonmagnetic materials, and layers to provide not just the disrupting microwaves but also the magnetic pushes and spatial manipulation (elimination and creation) in locations encouraging the fractal forms for fusion.

This is a combination of temporary plasma generation combined with electric and magnetic effects, fractal alignment and dimensional change, dimensional timing (including pretime “timing” of change); used to manipulate “space.”

Targeting fractal features to design and build real and “virtual” magnetic chambers with the help of the identified elements along with a solid reaction chamber and dense, perhaps liquid hydrogen.

Balancing means are critical as a KFE to do fusion.

The spark is the generation of offset neutrons or collapsible H+ neutron “hybrids” along with compression-oriented electron/positron pairs.

Space can be reduced by closing off the dimensions and concentrating the space with magnets and inverter magnets operating at 90 degrees to the electrical channeling charges for the protons and neutrons.

Desired CT states can be drawn through the chamber in various directions together or to sequentially create flows to target combinations that are compressive or decompressive for different CT levels; particularly proton collapse which requires collapsed t15 states and neutron pairing and balancing.

### Virtual or Low CT State Chambers

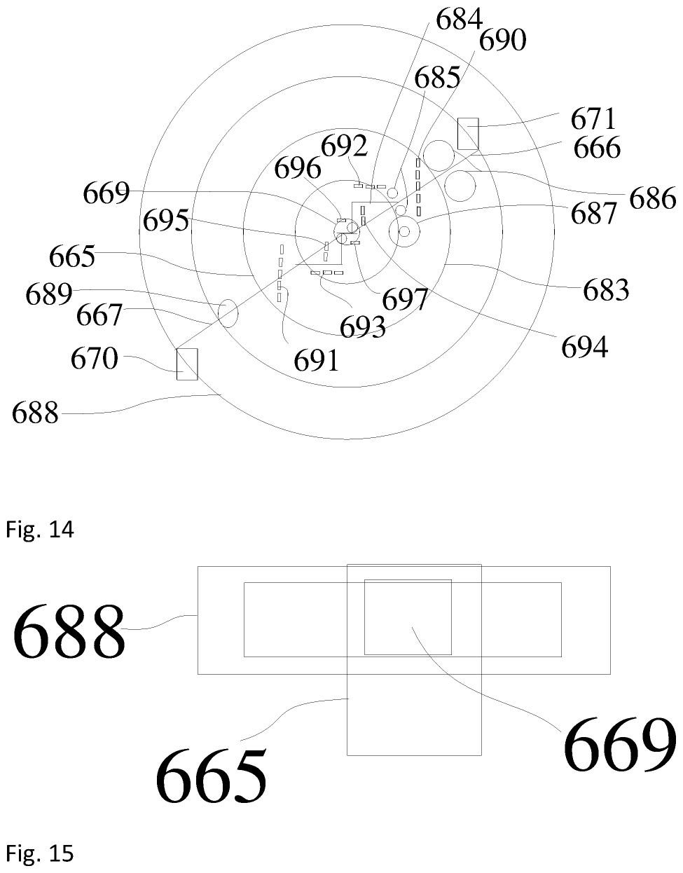

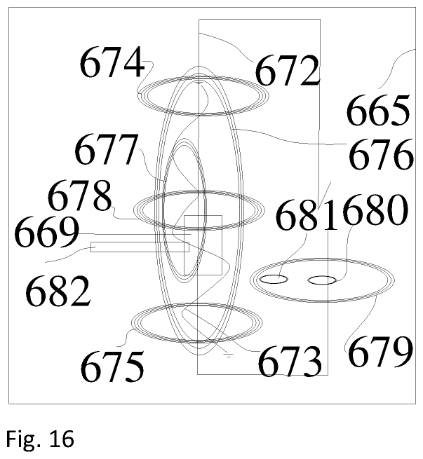

Along with a real chamber, a “virtual” chamber is shown in FIG. 16 and is relevant to understanding the reactor in FIGS. 14 and 15, the virtual chamber being adjustable around a reaction chamber 669 in FIG. 14. The reaction chamber 669 would hold a quantity of dense hydrogen for proton to neutron fusion or a neutron donor as set forth above.

The location of the fields and their crucial overlaps can be controlled by electricity used to activate the coils, the wave length and volume, that generate the fields from Magnets top magnet 674, bottom magnet 675, outer side magnet 676, center magnet 678 and inner side magnet 677; all shown as coiled wires which form electromagnets which can be controlled by the direction and extent of charge moving through the coils. Together these are used to limit the information in and out of reaction chamber 669.

Exterior magnet 679 contains inverter magnet 680 and second inverter magnet 679 can further channel the more detailed control of ct4t11 states into reaction chamber 669.

To activate the reaction shown below at least one Microwave generator 682 for creating plasma temporarily to be compressed, designed to maximize the targeted breaks in conjunction with the manipulating fields while minimizing unwanted impurities and maximizing any that might assist in the reaction desired and set or offset to assist in creating and utilizing the targeted locations. Here inverter magnets are those magnets creating inverter magnet fields; but also changing field features to make the reaction more efficient.

Another benefit of a virtual chamber is that by changing the “field generators” which are active, the chamber can focus alternately on the fpix or Mi characteristics of the stage of the fusion reaction and the CT states involved, particularly going from MI based for neutron arrangement encouragement (e.g. shaping along Mi based designs and balancing) to that for fpix for the design and balance of the proton core and electrons.

In addition to the M+ from the magnetic fields there is a first current carrier 672 and second current carrier 673 to facilitate the movement of electrons into the reaction chamber.

The reaction targeted is one or both of the following: 1) breaking up the paired ct4t15 states that make up protons and recombining those which are more compressive in nature, particularly those collapsed without an AuT positron (defined as a positron coming off one or more t15 states) 2) collapsing the paired ct4t12 states that make up the electron and AuT positron to collapse the proton-electron pair into a neutron. In either event, along with this is the need for balancing them as they are formed with protons balanced with electrons.

The process of breaking and recombining is shown although without the overlapping spiral form envisioned for ct4t16 protons, which above are just represented as broken ct4t15 states which are recombined as neutrons and then balanced with protons and electrons at the bottom; the two t16N neutrons and the method of balancing them is similar to the balancing discussed for protons with electrons. While only sample elements are shown, there would be large numbers of each element.

Since presumably protons are made up of two overlapping spirals of 5ct4t15 states, these can broke into the two spirals by targeting the fulcrum balancing the two together, the individual ct4t15 states can be broken out, particularly targeting those which have electrons coming off of them in an environment of ct4t15 states which do not have electrons coming off so that recombination can yield neutrons balanced around fulcrums instead of protons.

The various coils spin M+ states as e− information states travel through the wires charging them with pretime information in the form of the increased movement and resulting lower state transitions.

The variation of CT states to form virtual chambers allows for isolation of smaller CT states, ct4t11 at least, acting as a barrier to encroaching or leaking ct4t11 states or make sure a fluctuating mix has the right ct4t11 elements and inevitably having a more direct effect on ct4t10 states and hence lower states to control distances otherwise defined as vacuum.

The coils show the use of magnets within magnets and the ability to reposition those by charging sequential coils. The coils can be changed by changing the scale and direction of charge.

The small, adjustably aligned, single or Multiple alternate or combined internal coils to larger external coils or aligned with adjustably sized coils can be used to create the necessary CT environment, control and/or block CT state “leakage” in or out, locate points of CT state transition to create the fpix, MI, exponential or related spaces needed to get the desired results in reactions and fusion at the M+ state as well as at the electron state.

The focus in all transactions in this science is identifying and controlling the places where information exchange occurs. It is difficult to directly affect CT states below ct11, but they can be indirectly determined and ordered in such a way as to improve information exchange and, ultimately, moving up or down the chain of compression.

Another arrangement of coils and magnetic fields (F1 and F2 where the overlaps can be targeted and to move and eject atoms or (as shown here) ct4t11 states ct4t11H with a metal path as carl and without it on the right.

Field overlaps such as those at F1 and F2 focus on CT state interactions. An iron path connects CT state features, primarily here ct4t11 and ct4t12 features (electrons and magnetic features) used for fusion to bias compression.

Lowering pretime change, reducing spatial CT state concentrations “area” where exchange is occurring, increasing folded CT states for unfolded CT states.

Net compression or decompression of information in a system occurs at multiple levels, not just energy levels ct4t11-13. Movement of current and the magnetic driver (M+) is only one aspect of expansion in a system of pre-ct4t11 information states.

Every electric field includes magnetic components, the more dimensional circulation (as with transformers) the more you push out and accelerate the ct4t11 field. A transformer circuit is to create a series of ct4t11 “hurricanes” (ct4t11-H) which carry their pretime change to the other transformer which can be more efficiently placed at multiple locations around the stepped-up portion to capture the maximum number of these spun off CT states.

Iron cores do more than traditional alignment enhancement of the “fields,” in this case targeting the iron (or Ni or other magnetic carrier) with providing a surface for the travel of these ct4t11-H in the direction where they are most easily transferred to the secondary coil if the primary coil is the one from which they are generated.

FIG. 14 shows a top view and FIG. 15 a side view of a reaction mechanism incorporating the features to control positioning and collapse of Hydrogen. Spinning can help with the ability to offset CT states along with the use of multiple CT state generators, spinning being the most common form of repositioning about a reaction chamber discussed here, gravity as ct1 to ct2 transitions can be harnessed by using rotation and centrifugal motion about a fixed fulcrum and targeting these types of interactions are required to manipulate smaller CT states with larger phenomena within a matrix.

Virtual chamber 683 isolates the magnetic flow into and out of the containment as discussed in reference to FIG. 16, controlling the type of CT states in containment. The process is to replace traditional vacuum with gradually more compressed CT states. The charging mechanisms for the wires and electromagnets are not shown as they are known in the art.

One feature of the method is targeting the neutrons using MI spiral alignment and the protons and electrons using fpix type concentration and alignment successively or by layer and hence there is an overlapping MI spiral framework 684 which serves as a template for the neutrons to be guided into the appropriate MI alignment within reaction chamber 669. To bring the protons into position to stabilize neutrons present or formed there is a top proton canon 671 providing proton along a means to guide them into place, here the top channel 666 which may have a negative current flow to hold and carry the proton in plasma form potentially to one side of the reaction chamber MI spiral and a bottom proton canon 670 with a bottom channel 667 to bring another stream of protons in alignment to the other side of the overlapping portion of the overlapping spiral framework.

Drawing information directly from the twin spiral engines can collapse the core of ct4t11 states by the interaction of outer magnet pair 686, electric field generator 687 and Inner magnet pair 685 which can provide, alternatively and together with similar sets present within the reactor area 688 to provide the alignment in conjunction with or alternative to a virtual chamber of the type taught with reference to FIG. 16.

There is a proton accelerator means 689 and as shown above this can be like the outer magnetic pair 686 which can push the charged particles along the top and bottom channels 666 and 667.

While the exterior field generator is shown offset, it might be centered, and the number and positioning would vary depending on how to isolate the CT states and generate the desired matrices.

Plasma control means, here 5× power plasma generator top 690, 5×power plasma generator bottom 691, 3×plasma generator top 692 and 3×plasma generator bottom 693, 2×plasma generator top 694 and 2× plasma generator bottom 695 and 1×plasma generator top 696 within the reaction chamber partially and 1×plasma generator bottom 697 can be offset fractally as shown in conjunction with the template formed by item 684 to force concentration of the ions of the reaction and keep them increasingly focused while not maintaining them in a form which discourages compression but instead removes the space between them in the form of lower pretime states. The elements are used together at the concentration, force or dimensional level at successive concentrations for the different components of fusion (M+, electrons, protons, ct4t15 states, positrons, neutrons) at levels of 1, 3, 5, 7 or 7, 5, 3, 1 for fpix layouts to give pre-neutron alignment effects or levels of 1,2, 3, 5, 8 or 8, 5, 3, 2, 1 sequences to give neutron level effects along with template means guiding the elements into their appropriate places.

The process is to shape, virtually and otherwise, the reaction chamber to sequentially push highly compressed hydrogen into a more compressed state and provide a neutron backbone with proton stabilization in conjunction with fields to get the protons balanced with electrons.

The example of positioning here are magnetic pumps utilizing inverter magnets based on the particulate nature of M+ effects and electrical currents aligned with these or perpendicular (or other angle) relative to these for e− effects are envisioned for isolating the magnetic effects.

These effects can be enhanced by adding fractal shapes as with iron filings which can be manipulated in the field, neutron absorbing atoms like lead, neutron reactant atoms such as the boron example above, to hold bond neutrons during the operation of the virtual chambers.

Multiple electromagnetics are sequentially activated to move and hold matrices together or remove undesired CT states through folding, unfolding and CT state absorption and spew and control alignment of CT states for fusion or other processes along the fractal defined pathways.

FIG. 15 shows a side view of the largest components of FIG. 14.

As an example, a single inverter magnet is shown at the top, but the number and arrangement may be varied as may the overlapping spiral template for the goals of chemical reaction or fusion or fission. Different field magnets, such as the external field generator or inverter 2 (or combinations of these or at least these units) can be used taking advantage of position, relative mass and charge characteristics to get the alignment necessary or the clouds of desired CT states.

Spinning elements of the chamber and CT state generators (electric, magnetic, those generating larger particles) can be used to get additional closed field effects. We can see this working with spinning of centrifugal force of a wheel about a fulcrum fixed against the pull of gravity holding the wheel perpendicular to the normal alignment flat to the gravitational field.

One set of magnetics or electronics could be used to hold a particle in place while another set could be used to shrink the separation involved, electronics being capable of holding a particle, for example, while the magnetics were used to change lower CT state concentrations.

To do the sequential changes necessary for fusion, charges can be reversed to go back and forth in direction, back and forth in compression and decompression sequentially, stepwise in terms of fractal time (the quantum count) and distance and orientation (about the template spirals and fulcrums) and based on the number of the magnets, size, distance between positive and negative poles, spacing and relative strength these magnetic or electric fields to obtain the desired orientation.

Plasma plays a limited role. The concept is to break apart CT states to get the parts needed for subsequent compression, push these parts into the proper matrix and, for fusion, stabilizing using protons and electrons. To encourage this fractally positioned plasma generators can sequentially compress the fusion atoms, H2 (not shown) to maintain the spiral template at both the top and bottom simultaneously. At the very center the plasma generators may do two things, getting maximum compression at the point where the reaction is to occur in the reaction chamber and providing the stabilizing protons and electrons. The order of compression shown can be reversed although the concentration of states suggests the modeling above since there is less room for plasma generators and the accompanying decompression as you deal with more purified, more confined compressed features. The nature of the metals used to generate plasma can be changed to increase the compressive or decompressive features sought and this may be true within a group. Curvature of the group can also serve to lens the reaction to a closed location and to allow removal of the plasma heath that otherwise separates the neutron, proton and electron elements.

The arrangements of magnets, magnetic effects, plasma generators and plasma effects are to mimic and encourage fractal changes to function as a radial fractal engine, staging the different reactions, control the inverter magnet overlap and plasma generator extents and locations. The choices, both of which may be appropriate in different settings are the f-series (1,2, 3, 5, 8) and fpix series (1, −3, 5, −7) and the composites as well as exponential increases (2\hat{ }n) in force and number as well as giving curvature by scaling outer to inner multi-unit plasma generators to get the features of (1) collapse, (2) alignment and (3) balance required for fusion.

1, −3, 5, −7 changes suggest that using different charges from inside to outside (minimum to maximum compression) or to give curvature (e.g. from outer to inner or inner to outer the odd numbering ensuring there is a centering or fulcrum focus) in order to get fusion or to make it efficient. There are different levels of curvature, inverted and un-inverted (curved in or out) that are possible in this way using, for example, increased energy outside compared to inside, to create a curvature effect without changing alignment.

In addition to the fpix and f-series, there is also the possibility of varying the charge and arrangement to get the 2\hat{ }n type compression results in either the magnetic or plasma effects which might mean, in one example, a fairly weak burst of plasma generation in hydrogen liquid of 3 generators, followed by a fairly high burst twice as high at 5 generators, followed by one that is 4 times as high at 7 generators, possibly with the direction (compression/decompression of the burst reversed each time to give the positive and negative effects; again modeled around KFE for the steps required:

These work with or in place of the similarly designed magnets and/or inverter magnets to concentrate the elements and remove space between hydrogen atoms between the plasmag×1 above the spiral template top and the plasmag×1 below the spiral template bottom. A third and fourth set of plasma generators offset at 90 degrees with inverters offset, in the example at 90 degrees, to add to the compression by eliminating or reducing the dispersal of the fields by pretime states. Fifth and sixth sets of field generators offset at other angles can be used to further compress the elements.

Considerations include that magnetic effects are largely pretime meaning that when you use magnets to reduce or add space at various locations, the reductions are effective but largely offset by information moving into the system so you need to set this up so that you use exterior magnets to the inverter reaction space above to limit the amount of information that can come from the outside. The outer inverter shell would contain this arrangement, requiring the entire arrangement be within a shell of overlapping magnetic fields of the type discussed which is seen at large scale at intersecting magnetosphere/solar plasma between the earth and the sun, but taught as applicable to all intersecting fields, here used to isolate magnetic effects.

### Neutron Fusion

Helium fusion or stable proton to neutron fusion is a staged quantum process. The broad method is just to bring the neutrons into proximity to share information necessary to form the backbone, then protons must be brought in to form a core to stabilize the neutrons and electrons have to be brought in to stabilize the protons although the protons and electrons might be able to be brought in together.

Catalyst surfaces, for example Iron atom “gas” or “powder” to focus electromagnetic effects sufficiently close to neutrons to eliminate space (iron/ni/etc.) can be used. Using high energy (microwave level) photons to create paired mini pockets of plasma at the iron may be helpful in building fulcrum around which the neutrons can balance much like a spark in a spark plug except the size of the spark is exceedingly small. The level of energy while envisioned as plasma, might be substantially less, perhaps just small pockets or clouds of the targeted levels of CT states which are shared by fused neutrons. In spiral format it is an example of having a spiral format of application (1,2, 3, 5; 5, 3, 2, 1 pattern or shaped in spiral/overlapping spiral forms) to encourage fusion.

Because M+CT states are small, “environments” for fusion are the focus and not actual changes to individual neutrons or protons. The overlap of two fields or two different types of fields (plasma and magnetic fields are a good example) forms an energy exchange area. These fields should be modeled on (1) larger observable fractals where possible and (2) on CT state changes, that is the fundamental CT states and their overlap and the intervening smaller CT states to focus the space removal or energy exchange in the CT states (ct4t15 and ct4t12 states for collapsing protons into neutrons and ct4 neutrons to bring the neutrons together along with the lower information states acting as fulcrums and balancing features to assist in collapse and recombination.

Shaping and concentration at scales where we can work can be used to position states along a path of reaction defined by, for example, iron filings having widths or separations consistent with the desired transition sizes but at large scale to encourage the desired combinations, such as fulcrum of electromagnetic states on either side of neutron concentration at the sizes where direct impacts are difficult.

### Use of Fractal Models to Apply Large and Small Results

### Proton to Neutron Fusion

To get fusion the positron has to be balanced with a collapsed electron cloud and then pushed into the t15 mix along with balancing with other proton-electron pairs; the “subduction” of the t12 state of an electron layer into the proton layer at the positron 34 which would shift with the location of subduction.

The earthquake model suggests the subduction location is fixed, at least at the moment of subduction. While it can shift to location 45 but the slower shifts in the earth crust indicate this is not as common as it might otherwise appear. The model suggests that the mantle equates with the open area between the electron cloud of ct4t12 states 172 (primarily) and the inner shell of ct4t15 states 175.

Stabilizing plasma is contraindicated which lowers the complexity. Instead of containing fusion, the process is to focus on (1) compression substitutions to combine states that tend to stay compressed or (2) collapsing proton-positron-electron pairs and (3) providing an environment for alignment and combinations using the f-series pairs shown and confining and balancing these with the fpix series 1:1 (2), 3:3 (6) and 9:9 (18) pairing of protons and (4) fractal alignment on either side of a fulcrum of lower information states and (5) collapse around the fulcrum of the Neutrons with (6) balancing absorption and spew.

The elements balance about at least one AuT plasma fulcrum, a fulcrum at least partially of lower CT states about which higher CT states balance, involving fractal overlap and information sharing, folding in larger atoms adding stability.

Neutron backbones form a foundation for proton cores which stabilize the neutrons by providing information necessary for the neutron to continually “absorb and spew” information.

Proton to Neutron fusion requires ct4t15/ct4t12 folding around a stable ct15 backbone with supporting CT states along overlapping fractal lines absorbing and spewing to maintain the collapse of the ct4t12 states in pairs within paired ct4t15 states. Having “pre-paired” protons and neutrons (essentially hydrogen) ready to build balancing neutron backbone is easy to envision as a step in “cooling neutrons” separated by plasma generation and recombined with electromagnetics pulling the plasma away leaving the neutron backbone halves of whatever size more exposed to one another.

The process includes using exchange of CT state features between at least two matrices to remove non-compressive CT states, increasing compressive absorption in CT states, balancing ct4 states within the neutron backbone with ct4 transitional states, overlapping core ct4t15, balancing absorption and spew at each stage as well as the net release of excess spew in the form of decompression pretime information.

The CT state matrix can be imbued with compression tending KFE features, crudely referred to as being sequentially energized into plasma and de-energized (collapsed) about neutrons accelerated by lasers, magnets, mechanical compression, or other compression means preferably along KFE mandated fractal lines.

An example of generating plasma around neutrons is with microwaves through a conductor, contacting at the points of the conductor where balancing plasma is desired around an overlapping neutron core; pretime state changes excite the separation of protons and electrons from metals; but this is a process properly tied to information exchange and not non-fractal approximations using terms like energy or electricity even though those terms are measurable.

Projected fusion is where the elements are pushed together, shaped fusion is where the elements are in a cage which encourages stabilized alignment of the elements. These can be used together. Opposing like magnetic fields when viewed through this lens is more like the forces on windmills suspended on strings tied to their fulcrums within opposing “air masses” pushed by fans. One can see the difficulty of getting the fans in alignment, but it can also be plotted. The magnetic fans can have added lasers of higher ct4t12 states to provide virtual walls about which proton-electron balancing pairs can be held expanded or added to balance the end result.

Multiple and successive streams of CT states of a certain compression state can stabilize one another. Electrons can be concentrated and mixed with protons and then a central neutron core is added as the charged particles are concentrated and accelerated out to stabilize them. The protons are central to the electrons but having them start on the outside might be beneficial as they would pass through the ct4t11 clouds pulling everything together. Likewise, having a negative charge to the neutron tube especially towards the end might help to pull protons towards the neutrons which can be accelerated by having them pulled along by a strong electromagnetic accelerator and the separately accelerated protons (opposite charge) due to the shared absorption from the protons as the EM field is reduced.

Protons (P) can also be provided in the form of metallic generated plasma from microwave level energies through conducting wires making contact around a source of neutrons.

Mechanical (shaped as by spiral funneling, like what you see with water through a drain or with a chamber with screw pathways in the center or at the walls or both, possibly offset (oppositely spiraled and expanded to increase mixing), using electromechanical and sub-electromechanical (magnetic ct4t11) means to mimic these forms to concentrate the balanced matrix and organize multiple AuT plasmas at different levels. Facing plasma streams can replace the physical and virtual chambers in part with an offset to encourage rotational balance at the point where the two streams of information meet, with different plasma streams supplying the CT states for at least some of the “six steps of fusion” including stabilizing CT states or states to balance at each stage the process or encourage the continuation to the next stage.

The number and arrangement of the canon can be determined both by the CT states and the order in which they need to be added, the need to maintain the neutron backbone and plasma between neutrons in place and the fractal shape of the resulting atoms in addition to other KFE which includes the relative energies necessary to bring together the CT states to be fused.

Directed energy” or “plasma ignition” is analogous to concentrating states with high pretime change rates (tending towards gamma rays) from those with less pretime change (tending towards infrared).

KFE includes Neutron donors within the plasma distributed to encourage overlapping neutrons with surrounding protons and electrons on either side of stabilizing AuT plasma fulcrums.

### Fractal Fission,

The new view of fission is based on a neutron backbone supported by information exchanges with a balancing proton core which in turn is balanced by electrons. The energy comes from the dissolution of the unbalanced neutrons freed from the backbone by the separation of Potassium and Barium typically.

AuT modeling of Radon indicates a 5:4 break in one of the arms, suggesting the 2:10:18 relationship as each proton core is built and also where they can be targeted to break down.

Reaction chambers to maximize recovery have already been covered with FIGS. 8-11 showing how to target energy exchanges for Fission efficiency. To get fusion, you need to reverse this process, along with balanced folding and the two processes become relatively dissimilar quickly. Fission changes match those set out in the section on fractal maximizing mass/energy transfers, incorporating fractal overlays and fills, can have a specific application in fission reactors to manage the energy produced by the reactor. The invention can be used to (1) absorb heat generated by the reactor and use it to heat water to turn turbines, and (2) provide shielding for alpha and gamma rays to protect the environment.

In the first application, the fractal overlays and fills can be used to absorb heat generated by the fission reactions within the reactor. This absorbed heat can then be transferred to water, which can be used to turn turbines.

By using the fractal design, the fuel rods information exchange is optimized to enhance the fission reactions and increase energy production.

Fuel rod design heat exchanger (fpix (E) or MI (S)) overlapping or linear may transition between fpix and MI targeting (1) where energy release occurs, (2) the transition from MI to fpix modeling and collecting energy based on this dimensional exchange which is stepped transitions from one to the next.

To maximize absorption of energy from a thermal source the holes may be viewed as pipes seen in a cross section with the bigger pipes gradually getting smaller or the small pipes gradually getting bigger as the water or other coolant or heat transfer gas or liquid otherwise moves back and forth to maximize the heat transfer using fractal compression.

Note the use of 6 sized, 14 sided and rounded pipes to target multiple fractal features and reinforce absorption, in this case, possibly by having at the heat interface the flat six sided within the sphere inside of it. See AuT-fractal modeling with electromagnetic effects above)

Half circles can be curved at the cut end to maximize contact with a curved inner face or flattened if the inner face is squared. “Honeycomb” and fractal construction, especially six sided, or “Fullerene” modeling can be used in place of circles and spheres. Six-sided geometry originates because of the design of the proton core around a neutron backbone.

The extra “2” protons of Uranium (U235) (in addition to the 10-9-unit arms) are drawn “extracted” as a line with the number 2 labeling for clarity. Since we want to use fractal modeling to target the specifics of the energy release, this means the neutrons which ultimately dissolve to yield energy from the fission reaction, come from rebalancing the core from U235 to the Potassium/Barium pair.

The “energy” of the reaction comes from the breakup of the neutron backbone and possibly dissolving two freed neutrons which are no longer stabilized. The Helium, viewed as ejected as an Alpha wave according to the prior art, is probably too stable to be easily broken down. This means the neutrons which release the energy and can be targeted come from the larger balanced structure. While this type of fine-tuned targeting might seem out of reach, fractal math allows us to target the invisible at scale because transitions express themselves at scale meaning we can reduce radioactive waste and increase energy recovery using this type of modeling.

### A Prior Art Look at “Rays”

The extra “2” protons of Uranium (in addition to the 10-9-unit arms) are drawn “extracted” from being centered for clarity. One can think of these two protons as the protons which would otherwise stabilize the two dissolved neutrons as those two neutrons, soon to be dissolved, are pulled out from the proton core in the drawing of the “balanced” uranium proton core above. Fractal math suggests that the Helium (ejected as an Alpha wave according to the prior art) is too stable to be easily broken down and the resulting atoms, shown below, indicate the dissolved neutron come from the larger resulting atoms. The suggestion is that the extracted 2 protons and their associated neutron remain stable. The neutrons which ultimately dissolve to yield energy from the fission reaction, come from rebalancing the core from U235 to the Potassium/Barium pairing. Those soon to be dissolved neutrons, mentioned earlier, are a bundle of ct4t15 states. They decompress into a collection of ct4t12 and lower states which are viewed as energy based on their pretime change content.

### Chemistry

### Claimed Uses

Fractal models can be used to describe the development of plasma-based systems, including the fulcrums of lower state information between higher compression CT states, which can be utilized to drive chemical reactions and catalysis processes. The models can be applied to the development of materials such as atomic, molecular, alloy, and salt systems, and polymers, which can be optimized based on CT state transitions.

A subset of chemistry methods is the process of fractal categorization at the atomic level; categorized according to what could be called the “quantum fractal” method of this patent, which is a fractal method which uses fpix as a true quantum base, a bit.

The process is to redraw atoms and then molecules as interacting CT states and how these interact including how they interact when other CT states (typically referred to in the prior art as energy) are added.

### Catalyst

Catalyst action is a subset of chemistry, and the same process allows for maximizing interactions modeling transitions within a first matrix of pretime change giving rise to CT state sharing effects at higher compression states relative to at least one second matrix which is targeted to be affected by having these temporary CT state exchange locations.

This can be done by creating and Targeting shifting AuT plasma centers targeting the migration of information between plasma centers within a matrix and expansion or contraction of the plasma centers with a matrix to increase or decrease the ease of migration of information treating the plasma as a fractal hinge.

Shifting plasma cores within CT states; move/expanding/contracting (change relative size) core 623 to do work then core and where it is a Catalyst reaction plasma shift and using heat to expand plasma to allow shift to ease moving in and out of balance.

New designs of rare earths (RE) show that they are modeled around an expanded Neutron to Neutron plasma fulcrum. One improvement is to target this new design, as with other chemistry, to get better catalytic effects, not just of RE but also in all catalytic chemistry.

AuT redefined the atom, showing that atoms are bound by absorption of information by the neutron backbone which is stabilized by the proton core around it which is, in turn stabilized by electrons which are halved forming pairs along the same overlapping spiral either with positrons extending from the protons or by associated, bound atoms. Catalysts can be formed and their performance maximized by focusing on the expansion and contraction of at least AuT fulcrums within the catalyst so that as one is expanded, the information is drawn from the other contracting it and when balance is returned, the fulcrums, having performed their work in the expanded or contracted form (or both) has been returned to a balanced state. The discussion on Rare Earth elements shows how the AuT fulcrums are expanded in those multi-arm atomic states.

### Hydrogen Generation

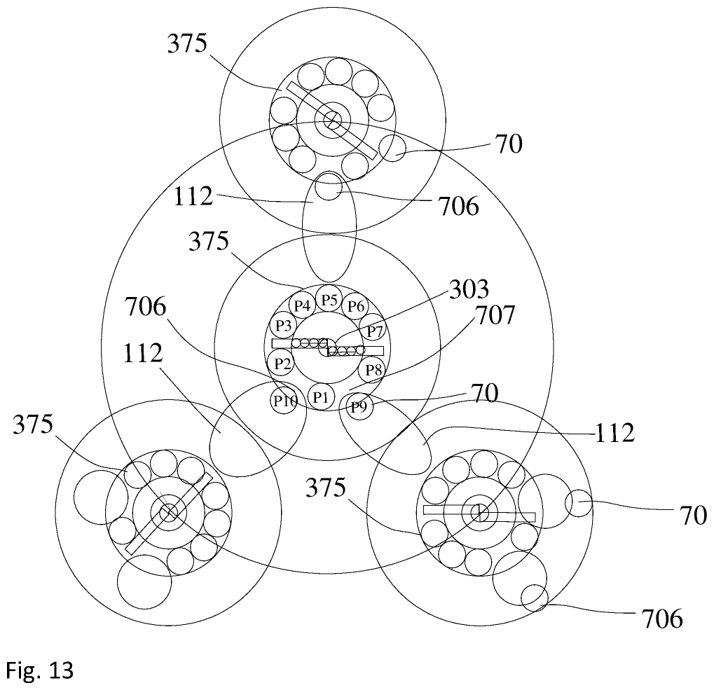

FIG. 13 covers how this modeling can improve the generation of hydrogen from water. In this figure the hydrogen-oxygen bonding is shown with the fractal form. What this shows is that water has a neutron backbone 303 with 8 protons numbered for the center Oxygen as P1 through P8. Between P1 and P2 is backbone-electron information sharing area 706. Between P1 and P8 is the second backbone-electron information sharing area 707 which gives water its unique shape. Above P1 and at P5 is electron-proton sharing between the oxygen-proton core 375 and a free hydrogen 70 which is weak since the extent of information sharing is limited between the proton 70 and the electron orbit 112 on either side with the area of the oxygen atoms designated as adjacent to the P5 proton of oxygen.

The bottom free hydrogen protons are designated as P9 and P10 because they can share information more directly with the neutron backbone through areas 706 and 707.

The expansion of water, for energy generation or in pistons or for propulsion can be viewed in terms of changing the bonds between the oxygen and the hydrogen at different energies interpreted as different amounts of pretime change, ct4t11 states and the resulting changes in the electron orbitals 112.

Electron sharing is viewed in terms of overlap as well as shared ct4t12 states as well as their associated ct4t11 composite magnetic effects (M+ to distinguish them from e−, the electron).

Because the model is fractal, fractal patterns exist. The specifics allow CT state targeting to strengthen one bond hydrogen type over the other. The bonds and electron orbitals are defined by fractal connections and can be targeted as fractals in order to get efficient changes. This can reflect the changing fractal numbers reflecting bonds, the changing size, ultimately the type and amount of CT states in the matrices. These fractal features change along fractal lines according to the fractal equations of AuT.

Fractal modeling removes the focus from charge to CT state sharing and the disruption of CT states to target separation of hydrogen bonds (remote) and proton sharing (close). This also means creating an environment conducive to O—O sharing and H—H sharing while also disrupting the alternative bonds as a single step or as sequential steps.

The location of where the charges are directed as shown can be based on the MI, fpix and 2\hat{ }n separation to mimic/replace/disrupt the different bonds based on their separation, the information shared, the base numbering and the other KFE.

H2O separation layouts are modeled with 0.75 of radius moves from outer to middle, 1.25 of radius moves from inner to 1rs inside.

This shows AuT or normal tesla valves with the length and angles defined by lines or curves defined by fpix or mi spirals or the length being defined by the 2\hat{ }n exchange areas.

The layout of (left) anodes and cathodes and (right) a cathode broken out in the same fashion.

The process can be reversed at some level and with some timing to encourage H2 formation from the dissolved water.

The two features are exponential change, balance, 2f(n), especially based on fpix for the Hydrogens and bonds, but moderated by the MI for the Oxygen.

FIG. 12 shows how these can be addressed by, for example, changing the energy, spacing, and/or spiral or areas according to fractal features. Positive Electrode 698 (cathode) and negative electrode 699 (anode) have a front side 700 facing the opposite electrode and rear side 701 so that positive electrode extensions 703 can be designed to pull in neutron, MI based materials on the front and on the rear use the same type characteristics to encouraged oxygen to oxygen bonding while the negative electrode extensions 702 target the fpix based hydrogens with 1, 3, 5 modeling and likewise encourage H2 bonding on the back so that one side can target disruption of bonding and the other side target encouraging bonding or one side can do both although they are separated here for clarity.

Exponential spacing, fpix spacing or MI spacing of multiple anodes and electrodes can be used to find the optimum relationship for different types of electrolysis.

Targeting hydrogen to hydrogen bonds involves fpix modeling. Targeting oxygen to hydrogen bonds involves fpix and MI modeling and oxygen to oxygen bonds involves fpix and MI but with more MI than the hydrogen oxygen bond which has one half operating with fpix (hydrogen) and the other operating with MI and balancing fpix (oxygen).

The shaping of the electrodes, e.g., round to match 2\hat{ }n, hexagonal to match fpix or MI transitions at different stages can be used.

There are multiple places to disconnect so designing for each, one is the hydrogen bonds which are remotely shared between oxygen, presumably a balance with a comparable “AuT-positron” equivalent coming off of the Oxygen atom; and the second is that sharing more directly with oxygen. This process can be effective at the surface of a catalyst.

Starting with an initial separation through introduction of one or more type of information followed by a change to a second frequency or concentration of information.

Another item is inserting an information state within the matrix and then exciting those inserted states.

Fractal modeling: Fractal modeling is a mathematical technique that can be used to describe the structure of complex systems. In the context of hydrogen separation, fractal modeling can be used to identify the specific bonds between oxygen and hydrogen that are most susceptible to disruption. This information can then be used to design a separation process that targets these bonds and produces high-purity hydrogen.

CT state targeting: CT states are a type of quantum state that is associated with the overlap of electron orbitals. In the context of hydrogen separation, CT state targeting can be used to strengthen the bonds between oxygen and hydrogen that are desired, while weakening the bonds that are not desired. This can be done by applying an electric field or a magnetic field to the water molecules.

O—O sharing and H—H sharing: O—O sharing and H—H sharing are two types of chemical bonds that can form between oxygen and hydrogen atoms. O—O sharing is a type of covalent bond, while H—H sharing is a type of hydrogen bond. Fractal modeling can be used to identify the specific conditions that are necessary for O—O sharing and H—H sharing to occur. This information can then be used to design a separation process that produces high-purity hydrogen.

The following are advantages:

Fractal modeling is a powerful tool that can be used to describe the structure of complex systems.

CT state targeting can be used to strengthen or weaken specific bonds between atoms.

O—O sharing and H—H sharing are two types of chemical bonds that can be used to produce high-purity hydrogen.

Overall, the hydrogen separation techniques can incorporate these KFE for separation whether in membranes, steam reforming steps or within atomic or molecular structures designed as catalysts to lower the energy necessary for high-purity hydrogen.

The use of fractal features in the design of the electrodes can help to improve the efficiency of the separation process. Fractals are self-similar patterns that repeat at different scales, and this property can be used to create electrodes with a high surface area to volume ratio. This increased surface area will allow for more efficient contact between the electrodes and the water molecules, which will lead to a more complete separation of the hydrogen and oxygen.

The use of different types of information can also be used to improve the efficiency of the separation process. For example, one type of information that could be used is the frequency of the light that is used to illuminate the water. The frequency of the light can be chosen to match the resonant frequency of the hydrogen bonds, which will make it easier to break these bonds and separate the hydrogen and oxygen.

Another way to improve the efficiency of the separation process is to insert an information state within the matrix. This can be done by using a laser to create a pattern of light within the water. The pattern of light can be used to create a specific energy state within the water, which will make it easier to separate the hydrogen and oxygen.

Hydrogen separation techniques involve the separation of hydrogen from other elements or compounds, such as water. In the figure mentioned, hydrogen is generated from water using a fractal model that targets the bonds between oxygen and hydrogen. Fractal modeling focuses on CT state sharing and disruption to separate hydrogen bonds and proton sharing.

Fractal patterns exist in the bonds and electron orbitals, which can be targeted to strengthen one type of hydrogen bond over the other. The fractal features change along fractal lines according to the fractal equations of AuT, allowing for efficient changes in the hydrogen bonds.

The location of where the charges are directed can be based on the MI, fpix, and 2\hat{ }n separation to mimic, replace, or disrupt different bonds based on their separation, information shared, base numbering, and other KFE.

Overall, hydrogen separation techniques involve targeting the bonds between hydrogen and other elements or compounds using fractal modeling to efficiently separate hydrogen bonds and proton sharing.

The separation of hydrogen from water using fractal modeling can be further optimized by manipulating the layout of the electrodes and the energy, spacing, and spiral or area according to fractal features. Electrodes with exponential or fractal spacing at different levels can be used to energize and separate hydrogen at different levels. The shaping of electrodes can also be used to match the transitions at different stages.

Multiple places to disconnect can be designed for, such as hydrogen bonds remotely shared between oxygen and sharing more directly with oxygen. This process can be effective at the surface of a catalyst.

To initiate separation, one or more types of information can be introduced followed by a change to a second frequency or concentration of information. Additionally, inserting an information state within the matrix and then exciting those inserted states can also be used.

Overall, manipulating the layout of electrodes and fractal features can optimize the separation of hydrogen from water and increase efficiency.

### Polymers

Composites are two dimensional layouts much like graphite and provide a good example of how to use large scale changes to manipulate atomic scale interactions. To counteract this, the goal is to create a flow of circulated resins by having the layup pushed into 3 dimensions (3 separate dimensions) as the resin sets, possibly through resin tubes going off in multiple dimensions, but more likely by the movement in three dimensions of the resin with plugs moving and likely screwing movement as with screw-shaped plugs during the cure or pulling plugs in the multiple dimensions where anti shear forces are desired.

Determined that shear strength failures resulted from the two-dimensional atomic features associated with polymer layups. Techniques like rotation fail to introduce sufficient three-dimensional interaction and modifications in static layups have also proved inadequate to date. To deal with these problems, the idea of fiber ropes and/or rotational inserts which may move into static buttons within a setting layup, a mold as the resin is beginning to set. These can be introduced utilizing forces pulling from within the mold using pullies outside or inside of the mold or pushing these from outside of the mold as shown in the two examples below. These may be throughout the mold or may be used as buttons or seals along key stress points.

### 2. Chemical Modeling:

A resin formulation that can be used on carbon fiber that can add 3-dimensional interactions to strengthen oven cured laminates and autoclave cured laminates is one claim. The goal is to have a vinyl ester bond with excellent shear properties to a carbon fiber at room temperature. This process has been applied already to resins which are modeled below.

There is a tested cross polymerization Example: Puncturing bags of cross polymerization resins with the plug, used in this example with cellulose within the weave, A portion of the layup above is a cellulose weave with puncturable bags of reactive interconnecting chemicals.

As shown in the example of cellulose weaves as an example, they can be interconnected utilizing hardened cellulose techniques by adding Si(OH)2CH3+B(OH)3+an acid catalyst, by way of example.

3. Dielectric Manipulation: AuT provides advanced electromagnetic modeling. Dielectric materials or insulators for different environments are documented, the advantage offered by AuT is the accurate understanding of how electric currents and magnetic fields interact.

The left drawing above is a view of electromechanics as modeled by AuT which is a different view of the results than the more traditional views shown to the right. The materials utilized may include internal high dielectric constant materials within the top, bottom and center layer(s) of the layup primarily to focus on any potential issues which arise from the three-dimensional transitions of the resulting composites.

Potential dielectrics involved include Polyimide and poly(ether imide); Fluorene polyester and cross-linked divinyltetramethyldisiloxane-bis(benzocyclobutene). Because of the unique ability to manipulate carbon atoms, aromatic films like Kapton PI which has commercial availability would be a good target.

One focus is to develop solutions to composite shear failure based on mechanical manipulation of mold layup. Secondary to this application are chemical solutions to the composite shear. In both of these cases, the process is to increase 3-dimensional cure matrices.

The physical strength of the hexagon-propane-hexagon core of resins is similar to the structure envisioned for atoms without neutron information sharing, at least not directly. Knowing more specifically how fractal strengths are developed with staggered compression and how the atomic components function across different atoms and molecules allows for incorporating cooperative structures and atomic links between the resins and mesh so that shear can be reduced.

The main difference between AuT and prior art lies in the ability to go further into the reaction process and to correctly model bonding ignoring the more traditional and largely inaccurate electron orbital model. Empirical knowledge in this area is decades ahead of theory and incorporating empirical knowledge into AuT science is a critical part of this.

Fiber ropes and/or rotational inserts which may move into static buttons “modeled” within a setting layup, a mold as the resin is beginning to set. It is important to look at these all as fibrous elements, although some solid pieces can be used. These can be introduced utilizing forces pulling from within the mold using pullies outside or inside of the mold or pushing these from outside of the mold as shown in the two examples below.

The inserts are filled with and made up of resin absorbing reinforcing material and are inserted during the layup process with unbroken walls which allow them to remain untreated as the rest of the layup is hardened. A flexible “loving” resin dispersal layer of the type shown in U.S. Pat. No. 6,508,974 (2003), and 6,203,749 (2001) is shown between the top and bottom layers and is positioned to disperse resin into the top and bottom unhardened insert. Also present are unbroken resin balls which serve the purpose of dispersing resin when broken to sections of the panel which are crushed, as shown below when the panel is shaped to the surface to be repaired so greatly that the loving resin dispersal layer cannot function for those portions of the panel, the crushing occurring as the panel is bent.

The opposite process is possible, using mechanical means to impart more dimension to chemical reactions. In this case adding fractally significant spiraling during the cure process in at least three dimensions within a primarily 2-dimensional polymer layup to extend three dimensional effects within the resulting part.

Atomic fractal design shows that lower CT states, including simple atoms, tend to bond in two dimensions and to eliminate the resulting shear in the third dimension, higher dimensional effects need to be integrated into these same molecular bonds in two dimensions to give them more dimensional effects.

Resins tend to bond in two dimensions which ensures that shear forces will be uneven. Through rotation gravitational effects can be minimized; but the nature of the polymers is two dimensional. While overlap and balance (about a mathematical fulcrum), folding and unfolding are ways to include three dimensional effects, if allowed to cure without some outside force, the shears will be present. Over a significant area, spiraling can be introduced to form a three-dimensional cure within the mold, what can be referred to as a fractally significant spiraling within the mold during the cure process.

This can be done by stirring the resin, weave, or both during the curing process with the greater the stir in multiple directions, the less the resulting shear although this must be balanced with allowing the resin to set as fully as possible. Minimizing the amount of resin disturbed is one way to minimize the defects possible in this process.

To enhance the dimensionality of polymer layups, various techniques involving mechanics and chemistry within the weave can be employed. One approach involves the use of cellulose porous plugs pushed into a mold during the curing process. These plugs can twist as they are inserted to impart vertical strength to the polymers. The objective is to induce two-dimensional polymer structures to buckle and fold into a third dimension, resembling fractal folding patterns.

A combination of vinyl resin and cellulose-based products allows for targeted manipulation of shear and electromagnetic properties through atomic modeling of AuT chemistry. By drawing and twisting these materials together during the curing process, while incorporating varying widths, threading, or layering, the desired balance, overlap, and folding effects can be achieved. Both dual and single resin activation approaches can be considered, utilizing different resins or reactants for bonding the main weave and the insert.

Additionally, hemp or rope made from cellulose materials can be employed as reinforcements to guide the cellulose weave through spirals within the primary layup. These concepts are supported mathematically in the proof, while the accompanying illustrations further exemplify the practical implementation of these techniques.

These methodologies provide a scientific basis for incorporating additional dimensions into polymer layups, allowing for the creation of intricate structures with enhanced properties. Adding dimension to polymers can be achieved through a combination of mechanics and chemistry within the layup weaves. This process involves utilizing the same or alternate materials, such as cellulose, in a three-dimensional configuration. During the curing stage, mechanical stirring is employed initially and then halted to create spirally relevant forms in three dimensions within the mold. This is accomplished by winding and unwinding lines around hardened areas in the weave, guiding them along desired pathways to leave the cured resin with the desired forms.

Alternatively, dimension can be added by extruding specific features necessary for significant spiraling (balance, overlap, and folding) within the curing structure. The width of the string is not a limiting factor, allowing for the use of sheets composed of absorbent threads pulled around pivots and to change the dimensional direction of travel. Magnetic particles within the matrix can also be utilized, replacing the lines and pivots by leveraging magnetism.

By incorporating the features of fractal construction in multiple dimensions, such as overlap, folding, and balance, along fractally relevant lines in the resin, the desired effect is maximized. This approach effectively reduces shear and optimizes the utilization of fractal extensions across different dimensions.

In addition to the methods mentioned earlier, there are other ways to incorporate multiple dimensional frameworks into polymer layups during the hardening process. Here are examples:

Fiber Alignment: By aligning fibers in specific orientations within the polymer matrix, multidimensional frameworks can be achieved. This can be accomplished by controlling the fiber direction during the layup or using techniques such as electrospinning to align fibers before embedding them in the polymer.

Mold Design: Utilizing molds with intricate geometries or patterns can introduce additional dimensions to the polymer layup. The design of the mold can be customized to create specific three-dimensional structures or complex surface features.

Layer-by-Layer Assembly: Employing a layer-by-layer assembly technique allows for the precise arrangement of materials with different properties and dimensions. By sequentially stacking and bonding layers, multidimensional frameworks can be constructed within the polymer structure.

Additive Manufacturing: Techniques like 3D printing or additive manufacturing enable the creation of complex structures with multiple dimensions. By selectively depositing and curing polymer materials layer by layer, intricate and customizable frameworks can be achieved.

Templating: Using sacrificial or removable templates within the polymer layup offers a way to introduce multiple dimensions. The template material can be dissolved or removed after the curing process, leaving behind voids or channels that contribute to the multidimensional framework.

Surface Modification: Modifying the surface of the polymer matrix during the curing process can create multidimensional structures. Techniques such as laser ablation or chemical etching can be employed to introduce patterns or textures, adding an extra dimension to the overall layup.

These approaches provide alternative methods to incorporate multidimensional frameworks into polymer layups, allowing for enhanced properties, functionality, and structural complexity in the final material.

This is preferably done throughout the mold, but particularly in the areas where shear is experienced and at different points and different angles to prevent weakness in one area and in one dimension.

The same process can be used to incorporate electronic features tied to fractal designs into the matrix of a curing polymer.

### Quantum Computing

To accomplish the goals, we compare how quantum processors approach probability modeling with fractal quantum change. In using pretime change, it need not be exclusive, but instead can be used to correct probability features of pretime computing to make the results more useful.

Time is stop-frame animation based on changes in location of fractal pretime information states.⁶ The model of information physics used to quantify QC processors is appropriate to information (programming) based inquiries. Electrons are made up of photons in this scenario and the rotation of photon elements, even smaller pretime states than photons, around those photons when viewed from the standpoint of time appear as waves. Quantum computers mimic pretime computing using these pretime locations, but instead of using specific pretime locations to do computing, quantum computers use approximations based on probability. The resulting calculations have errors which limit the utility. If pretime positions are used, accurate results would be reached. Probabilistic results can be moderated with approximations of pretime positions.

Not all calculated positions need be precise. Instead the pretime change analysis can be used to correct errors in probability modeling.

Qubit behavior can be predicted by analyzing pretime information state changes and information physics.

To achieve our objectives, we propose the following research activities:

By identifying the key factors that limit the performance of quantum computers and break them down into fractal components (or fractal equivalents which need to be substituted for those key factors) and using the mathematics of pretime dimensional change leading to post-time qubit and time related transitions, to reduce errors and get as close as possible to true “pretime” computing.

Noise is eliminated by coincidental or focused use of the concept of pretime computing to redefine our understanding of time and its role in quantum computing. Pretime information states, which are made up of photons and even smaller pretime states, can be used to predict the behavior of electrons.

Quantum computers perform pretime computing, but approximations based on probabilities are used instead of the more precise pretime locations.

Through a reverse process, we can use quantum fractal algorithms and then look at the quantum computing results to find corrections, basically using known fractal results to correct the quantum errors, or in this case to see how closely probability-based devices come to getting accurate results. ⁶ “There are fractal codes that contain the laws of creation and the more we understand these laws, the more we can apply the harmony that manifests in nature as a more powerful force in our own lives.” Jonathan Quintin https://sacredgeometryweb.com/jonathan-quintin-what-is-sacred-geometry/

This is an example of using a fractal framework for assessing the utility of quantum processors.

QC relies on true randomness. Qubits (electrons for example) are composites of fpix bits, fractal components changing according to set rules which occur in a pretime environment. Errors can be corrected by applying pretime analysis of the changing bits allowing the speed of quantum computing with a “correcting” factor tied to the pretime changes in position which are otherwise relegated to random approximation. A crude attempt at using Fibonacci spirals had positive results in Quantum Computing. These crude experiments highlight the difference between understanding cause and effect and just recognizing the effect; but it shows the science can be used to manipulate qubits.

### AI Modeling Using AuT

AuT provides fractal modeling (AuT) which can be used to define any features at any level of dimensional existence based on fractals effects within the matrix under consideration. All AI is a function of algorithms and at their base is the same math that gives rise to all other things, adapted as shown in the various applications here, for the particular use at the scales and rates of change that are applicable.

In addition, the modeling will “marginalize” what is viewed as junk data, data without clear fractal features. Since today's junk is tomorrow's treasure, non-fractal data must be categorized so that if its future value it is not lost. Blockchain features will be built in to allow marginalized and primary data corroboration and ranking, initially tied to commentary and citations using the data.

With AI, content is contextualized and personalized by focusing on key fractal elements and outgrowths from key elements.

The increasing demand for process automation and optimization, research, data analysis, data interpretation, and demands for increased efficiency in the industries served by this data is discussed below. Fractal modeling provides a clear system based on fractal mathematics and applies this to provide fractal cross-referencing and output based on the fractal origins and underpinnings of data being examined. The challenge is not to define the basic mathematics (AuT), but to stretch AuT to the more complex systems of atomic interactions, molecular biology and the large resulting structures. AI is recognizing patterns so that the flow of data can be managed and using these patterns to work with the data. The math developed allows for fractal patterns to be used as patterns, in many cases requiring non-fractal data to be categorized based on the fractals within it.

The method includes: 1) identifying the fractal patterns which should be associated with the data and (2) looking for fractal patterns at all levels (energy, pre-energy, structural, component, environment, etc. (3) processing at least by categorization the data based on fractal modeling. In chemistry fractal components and reactivity elements have been identified based on confirmed, but unrefined fractal mathematical features. These features are the baseline fractal structure which can tie everything together to avoid chaos.

In the software, Content will be contextualized and personalized by focusing on the existence and function of fractal elements. Blockchain features will be built in to allow marginalized and primary data corroboration and ranking.

The challenge is not to define the basic mathematics, but to stretch the mathematics already developed to the more complex systems of reactions, molecular biology and the large resulting structures and to refine the mathematics for other structures.

Specific factors will be (1) Management: Since all results fall into a category of fractal science (which defines space, time, force, the atom, molecules up to black holes) data can be categorized based on fractal structure and function;

(2) Organizing historical data around fractal mathematics at the core of dimensional features, including function;

(3) There is no “unstructured metadata” because all dimensional features of the universe fall into a category of fractal math; and fractal math necessarily pigeonholes data in one or more fractal categories and the features which join the data based dimensional features are themselves fractal.

(5) The ability to connect distributed environment by virtue of fractal changes at different levels means that the combination of different dimensional or compression (CT) states and the resulting changes (reactions to yield different matrix) can be categorized, it doesn't mean the process is simple.

(7) Heterogeneous means that the common fractal link has to be determined so that the heterogeneous features are broken down into fractal elements. A fractal system cross reference to non-fractal interpretations is a complex undertaking because non-fractal interpretations have to be reinterpreted to show their fractal elements.

All systems can be based on fractal elements common to all dimensional features and treated as static with change being separated into fuse changes which are not seen and transitions between compression (CT) states. Dynamic systems can be categorized based on initial fractal matrix, the interaction of fractal elements, pretime change, absorption and spew between the different matrix and resulting fractal matrix.

This allows energy, even time, to be viewed as just another fractal change within a quantum fractal system, manifestations of change including those that result in energy changes can be treated as fractal matrix changes reflecting average pretime change.

Facial, body recognition; but more importantly prediction tied to alignment and choices inherent in the dual, overlapping spirals at different levels of compression targeting fractal features according to the math of AuT. Computational programming is tied to algorithms. While applications are more varied and diverse rendering AuT irrelevant to most, when you focus on “basic research aimed at having the broadest possible impact, the development of computational methods should include an emphasis on theoretical underpinnings, on rigorous convergence analysis, and on establishing provable bounds for approximation methods” you are ultimately getting to a foundation which is defined by AuT in terms of practical applications as opposed to the equally important human applications. Practical applications, the movement and interaction of both large- and small-scale phenomena are AuT centric determinations.

This is a technique for improving natural language processing by incorporating principles of the quantum entanglement of a linear set of fractal solutions. The technique uses the KFE involved in fractals applied to:
Self-similarity: the pattern is replicated at different scales and is similar to itself in all scales.
Iteration: the pattern is created through a repetitive process.
Scaling: the pattern is scalable and can be enlarged or reduced without losing its essential shape or character.
Fractal dimension: the pattern has a non-integer dimension that is between its topological and Euclidean dimensions.
Complexity: the pattern is complex, and its complexity increases with each iteration.
Chaos: the pattern exhibits deterministic chaos, meaning it is highly sensitive to initial conditions and displays unpredictable behavior.
Non-linearity: the pattern is nonlinear and exhibits different properties from those of linear systems.
Symmetry: the pattern has multiple symmetries, including self-symmetry, rotational symmetry, and reflective symmetry.
Attractors: the pattern has strange attractors, which are points or sets of points in its phase space that the system is attracted to over time.
Multifractality: the pattern exhibits multifractal properties, meaning its fractal dimension varies with location or scale.
Self-organization: the pattern emerges from the interaction of its components, and the system self-organizes to reach a stable state.
Power law distribution: the pattern exhibits a power law distribution of its properties, meaning the frequency of an event or magnitude of a property is inversely proportional to its size or rank.

### Climate Change

Harnessing the earth's magnetic and electric cores can be done with very strong magnetic fields strategically placed at or near the surface, possibly using existing spatial fields in space or locating the same in space, as for example to steer the electrical currents to specific locations from which they can be tapped therefore allowing further effects on the magnetosphere and energy transitions in the earth to deal with titanic events such as earthquakes and volcanoes I the same basic fashion taught for electro-magnetic effects but at scale. While these are extreme applications which are theoretically possible, but requiring extraordinary resource allocation, they bear inclusion because the survival of humanity apparently rests with these technologies which can also be used to generate energy at levels exponentially greater than fusion.

In the magnetosphere information is exchanged and transferred to the plasma coming from the sun and the combination moves around the earth protecting us from the full effects. The movement of this transition point depends on the relative strength of the two fields (technically the pretime change within the two sets of CT states) and this is why we can affect the temperature of things from a remote location by changing the pretime change in one field or the other. For climate change this involves enormous changes, but for purposes of effecting fusion, these are relatively easily targeted changes.

The nature of absorption and spew of t12 is relatively simple to understand, but complex in terms of analysis. The earths ct5 core absorbs and spews information, much of it staying within the core and the other layers of the earth as heat, but enough escapes as ct4t11 states to create the electromagnetic shield that protects the earth from the solar winds. The electric component is absorbed, being the larger T12 component (ct4t12) but the much smaller magnetic ct4t11 component escapes in sufficient amounts to energize the magnetosphere. Because they are related, the t12 component can be focused, pulled in a direction where it can be extracted and used to control titanic events utilizing the accessible t11 states.

Folding has been determined from empirical observations to be the effect of the next higher state having overlapping f-series spirals around which the lower states. Spherical effects increase with net compressive effects, net folding, within the overall structure, but this is proof of the ability to take these steps.

As climate change becomes an increasing problem, the ability to use deterrence instead of force will change detracting resources from the development of new systems and degrading the effectiveness of systems that are used up, not to mention the demands of manpower. Controlling climate change is much more effective and if it can include generation of energy at scales heretofore unimaginable, then so much the better. The earth's core has a generator which is exponentially greater than fusion.

To control climate, we need a force exponentially higher than what we have on earth and AuT provides that using the ct5 (think black hole) core of the earth. In this case the ct5 force (which incidentally includes lower CT state changes at exponential levels even though incidental) is concentrated at the black hole core, but the effects stretch out and create the magnetosphere impossible without this core of extremely high folding and unfolding energy.

The earth is a fractal equivalent of an atom or galaxy and using observations of these more easily observed fractals, it is possible with great precision to map the fractal structure of the earth and its relationship to earthquakes and other titanic phenomena.

Model: It is counter-intuitive to think of the earth, a hard ball of varying molten layers with a coating of water, as an atomic equivalent or a galactic equivalent. Nevertheless, equivalence is required by fractal mathematics. Using equivalence and the ability to determine field key fractal elements a wire or its equivalent can be put in the earth's magnetosphere and if the charge is drawn off, giving energy, then it can have effects.

The earth's magnetic field shifts. Either 1) the ct5 core rotates or 2) the core reaches an inflection point reversing the absorption and spew of the ct5 engines or stopping it as it reverses. Even this is subject to control because the system between the earth and moon cores can be controlled, in theory using the same mechanisms on either body.

KFE can be used to focus on the entire solar system as a matrix with sub-matrices.

Channeling is done the same way, wires above the main axis would be repeated over and over again coming from the earth's backbone to the surface and would be curved due to the dimensional effects which originate at the backbone. A magnet reflects the underlying engine, having two sides which operate to net out the overlapping spiral backbones at the center. The core energizes the layers immediately above it which in turn transfer energy to other layers. The ct4t11 and ct412 type states are absorbed, the magnetosphere is evidence that significant energy radiates outward both along quasi electrical and magnetic fractal lines as shown in the detail of magnets and therefore along equivalent arms extending both electrically and magnetically (e− and M+) and perpendicular with both absorption and spew lines and in this case it appears most of the electrical is absorbed and dispersed within the earth, but much of the M+ continues outward until it interacts (magnetic repulsion creating space as taught herein) when it comes into contact with the same features radiating from the sun which in turn exchange information to give angular momentum to the solar wind so that it moves around the earth. There is no reason why this phenomenon cannot be concentrated at the surface of the earth to energize electrons there to create currents or tapped along conducting existing, or supplemented by modification, features of the earth as to the primary or secondarily charged lines of electric (e−) or magnetic (M+) movement.

The method without fractal modeling is changing the actual or effective rotation of the earth as by creating additional rotation at the poles or other points with EM effects can be used to increase the exchange of pretime exchange between the earth and the sun. Absorption and spew between the earth and moon can be addressed in the same way.

Another KFE is the balance of absorption and spew at different points, while the AuT engines are a clear point, the other areas where absorption or spew occurs can also be targeted. KFE includes concentrations along flattening lines and expansion along a disc of 2-dimensions to drain energy from volcanoes and earthquakes is possible.

For the earth, running wires from one engine to the next getting as close as possible to the engine itself with the conductors. uncovering or changing to a less absorbing layer between the internal source and where it needs to be focused is a methodology for capturing trapped electrical charge as well as harnessing the magnetic portion for energy and climate control.

Ct5 is fusion energy, to tap ct6 energy you would have to capture a black hole. AuT suggests that we have that blackhole, multiple black holes, under our feet. If the earth has a ct5 core, there would be around it ct5tx (1-32) states. The PTE is only the first 2 of these (ct5t1 and ct5t2), the others being unstable except under the concentrations seen inside the earth and perhaps at ct5t16 or ct5t30, the compression in the earth stops; but the process set out for extracting energy from its folding and unfolding remains.

The energy still flows, but it does not generate a moving current although in the atmosphere and in deep space it interacts with the atmosphere and solar wind charges flow to impart energy and this effect is what needs to be repeated at the surface of the earth to draw out the energy or more specifically to get it directed so that it can be used.

A transformer can be used to change voltage and current, a rectifier circuit can be used to take ac current and transform it into dc current, a process which needs to be reversed here. An inverter converts dc to ac. This process can be used in conjunction with the dc flow of the earth's black hole core to create a useful current and to change the pattern of flow of some part of the magnetic power to change the output in a tangible way weatherwise, possibly moving the point at which it interacts with the solar wind or atmosphere or both. If the aurora is the solar wind getting through to the air, then you have an interplay of multiple parts, the earth where we can draw off current, the atmosphere where the solar winds initially impact, the place in space where the direct current of the earth interacts with the solar wind . . . is that part of an inverter? These are all places where these effects can be manipulated. This would allow not only better energy derivation from the earth as a battery/magnet; but also, to drain energy where it is dangerous such as pressure built at fault lines and at volcanos.

Competing reasons of (1) drawing off pretime change as electricity and (2) increasing or decreasing or reshaping the magnetosphere to get desired effects, primarily heating and cooling, but also other operations including drawing off information considered dangerous or concentrating information at various points within the earth. It is worth noting that the concept of climate control (vs energy generation) might include charging the coils from external sources.

### 2. Categorization

Using fractals with data science to modify AI software to maximize the accessibility, organization, and value of empirical data. Dealing with the complex variations such as what appears essentially infinite variation in life requires AI and AI requires a base structure, underlying algorithms which can tie everything together to avoid chaos. We can cross-characterize existing, empirical non-fractal data within sub-atomic, atomic, chemical, and biological data with fractal data required by the “AuT” Fractal modeling used to define any features at any level of dimensional existence based on fractals effects within the matrices under consideration.

Categorization is a broad way of describing design by breaking into fractal elements one or more of the following: chemistry (reaction; fuel; carbon capture; rare earth combinations) based on fractal chemical and molecular structure of the at least one matrix; biology (DNA, ATP, biological design, and function); physics including titanic event prediction and control; quantum gravity and time, black holes, dark energy, wave particle duality, etc.); Energy capture, generation, and storage; fission; Quantum Computing using the non-pretime change and pretime changing with non-pretime changing or amount of pretime change to deliver or withhold information; Concentrating pretime elements; qubit function and design utilizing key fractal elements (KFE).

CT states are an example of KFE as is quantum change which with the CT states creates pretime change for relative work.

Fractal modeling “integrate(s) multidisciplinary” information because the same fractal model applies to all dimensional features of the universe and even pre-dimensional features which energize the universe.

AuT includes treating AuT fpix changes as plus and minus. There are multiple ways in the electronic spectrum where the plus or minus result can be sampled or applied with fuse length, compression vs decompression; abs vs spew; pretime and post time change.

The absolute definition of change at ct1 is a quantum count common to all points in the universe, “absolute time” or more accurately “absolute change” or “AuT dimensional change.”

This allows energy, even time, to be viewed as just another fractal change within a quantum fractal system. Despite exponential complexity at different compression levels, fractal means there are equivalents at every level Larger atoms mimic galaxies in structure a concept which is the result of a mathematical analysis, not theory.

All of the different manifestations of change and those that result in energy changes can be treated as fractal matrix changes reflecting average pretime change.

The work includes: 1) identifying the fractal patterns which should be associated with the data and (2) looking for fractal patterns at all levels (energy, pre-energy, structural, component, environment, etc.) (3) processing at least by categorization the data based on fractal modeling. In chemistry fractal components and reactivity elements have been identified based on confirmed, but unrefined fractal mathematical features. These features are the baseline fractal structure which can tie everything together to avoid chaos.

### In the Software,

### Communication and Time

AuT involves using the relationships of CT state exchange along KFE features of information capture and release to more efficiently interpret results and increase post time speeds by concentrating on pretime CT states which allow for more instantaneous communication and computing.

Quantum measurements include replacing Planck length with quantum CT state changes at at least one CT state compression level. The method includes tracking 1) absolute time and 2) utilizing the rate of absolute change for time keeping, reconciling electromagnetic time (EMT) with absolute time, using quantum change to determine pretime wave characteristics, and converting existing clock models to quantum change some of which affect time and some of which are pretime.

Perceived Time is stop-frame animation effects, changes are in fractal building blocks of electrons, ct412 states which, in turn, are made up of 10 ct4t11 states, typically viewed as photons. The electron ct4t12 states and lower components (ct4t11 states) are in multiple locations and states for any measurable time and appear relativistic but only from a perspective of time. By dimensional analysis they remain Newtonian. Focusing on CT state pretime locations, quantum computing is done without or with at least less “probability” which suggests a “false randomness” which is replaced with specific “pretime locational or net AuT state change where AuT state changes are the positive or negative states in which ct1 states find themselves which are reflected in fractally significant positive or negative states in higher compression states. Pretime locations are locations where a particle exists from a pretime environment as seen from the perspective of time. Since these can be significant for any period of time (practical measurements of 10\hat{ }44 of these changes per second) pretime change computing is exponentially higher than with other computers modes.

### Time Dilation

1) Time Dilation: The ratio of pre-ct4t13 states passing within a ct4-ct5 transitional state to pre-ct4t13 states (PTS) changing outside of the ct4-ct5 transitional state is a time dilation ratio. This ratio of PTS into PTS moving in and out is the source of velocity time dilation. The movement of PTS within alter the arrangement of the wave states captured between the proton and the electron altering the history of points within the transitional state and the comparison of one collection of points to subsequent arrangements of the same points creates history.

### 2) Chapter 71 Relativity

To understand this, we start with fuse length and folding. At the edges of the universe the fuse length for newly created particles is 2 counts, then 4, the 6, and so on. At the center of the galaxy, the fuse lengths are older, on a scale of 10\hat{ }50 counts. Things at the center of the universe look amazingly stagnant, things at the edges change so fast they are pretime.

Two things happen with acceleration. 1) time slows down and 2) length increases. Time is change at the ct4t10-13 range, everything before that is pretime change that gives rise to time. More pretime change, less time since the pretime change is just more movement relative to post-time change. Acceleration is the change rate between time and pretime changes. Everything changing faster is invisible, everything changing slower is visible from the standpoint of time. All points exist frozen between quantum counts, having neither time nor speed. But as the quantum count increases certain points change and up to the ct4t12 state, these changes are acceleration, whether they are compressive or decompressive.

If you take two Matrix A (Still 1) and B (Still) which are identical and place them equal distance from the center of the universe we can see how this works. As change occurs within the matrix at the pretime level relative to the center (in this case accelerating away) there is more pretime change so the time within the point begins to change slower.

Likewise, the more solid part of the matrix begins to expand from the standpoint of time because each point that is changing in a pretime environment appears to be more places at once, hence appearing to assume more space, the item lengthens.

Acceleration has two components. 1) unfolding or folding change relative to a universal center and 2) pretime change compared to post-time change. Whether you accelerate towards or away from the center, the amount of ct1 change increases so that more of the change is pretime slowing down time while increasing the change within the system.

Gravity can be seen as pulling CT states within the matrix represented by the larger circle. Put another way, gravity is a net increase in folding. The more change occurs in compressed matrix (ct4t12 and above) the more the change is visible from the standpoint of time. With sufficient folding, since more of the ct1 states are part of CT states that are higher than ct4t12, the slower time goes for a given set of point because less of them are changing pretime creating more frames.

The changes expanding and contracting the size of the matrix appear as work and this is why we see energy as energy.

Dimension is also affected. As an increasing number of the ct2s straighten the entire matrix begins to flatten out. At an inflection point ct4 breaks down first from neutrons to partially filled states (e.g., electrons and protons) and then into energy.

### Conclusion:

Key Fractal Elements (KFE) can be used to improve the performance of a variety of technologies. KFE are a type of mathematical structure that can be found in nature, and they have been shown to have a number of beneficial properties. For example, KFE can be used to improve the efficiency of energy conversion, the accuracy of signal processing, and the stability of quantum systems. KFE enables increased energy efficiency, improved energy capture and dispersal, and enhanced energy management techniques, enhances reaction kinetics, allows for a way for CT states to be interpreted in a manner that increases reaction selectivity; optimize fission in a manner that improves product yields; allows for the design of molecules and polymer structure formation and improves polymer properties. KFE can be used to interpret or categorize matrices of CT states; for categorization, prediction, manipulation, and designing radiation matrices for various applications including electronics, solar, thermal, fusion, and radioactive energy use, capture, and dispersal; to modify frequency-based systems, thereby increasing the efficiency of energy generation, transmission, utilization, and storage, while enhancing overall accuracy of results; to leverage base transitions as KFE elements to govern matrix composition and interaction, encompassing spatial, energy, atomic, chemical, electrical, biological, and large structures, applicable across a range of CT states; to efficiently compress or decompress CT states and form matrices of CT states, facilitating desired dimensional variations; to design matrices based on KFE concepts, considering time as CT state dimensional change, energy as CT state change, and the structural aspects of neutron backbones, proton cores, and electron clouds, enabling control over energy release, redirection, and absorption; and design includes the design and manipulation of molecules, including the expansive or contractive features and orientation of atomic or molecular matrices throughout reactions as CT state matrix change occurs; to control the absorption and spew of CT state exchanges within matrices by strategically shaping reaction chamber parts, adjusting injector jets, and exhaust systems; to react chemicals, including fuels, for enhanced matrix changes, maximizing the release of pretime informational change, and optimizing energy utilization and to target AuT plasma, stepped transitions, categorization, chemistry, biology, and other relevant features, thereby improving processes in any undertaking.

The remarkable potential of key fractal elements (KFE) to revolutionize various industries cannot be overlooked. Through the integration of KFE in fusion, fission, chemistry, polymer chemistry, hydrogen extraction, electronics, batteries, quantum computing, and AI, we can achieve unprecedented advancements and reshape the landscape of these fields. The presented argument for the issuance of a patent for the application of KFE highlights the transformative impact it can have on energy generation, materials science, computational systems, and more. Embracing KFE unlocks a new era of innovation, sustainability, and efficiency, shaping a future where the boundaries of what is possible are pushed ever further.

1. A method for improving processes in any undertaking utilizing Key Fractal Elements (KFE), from the group comprising (1) Applying KFE to affect change in CT states and interpret or categorize matrices of CT states (2) Utilizing KFE elements for categorization, prediction, manipulation, and designing radiation matrices for various applications including electronics, solar, thermal, fusion, and radioactive energy use, capture, and dispersal (3) Employing KFE to modify frequency-based systems, thereby increasing the efficiency of energy generation, transmission, utilization, and storage, while enhancing overall accuracy of results (4) Leveraging base transitions as KFE elements to govern matrix composition and interaction, encompassing spatial, energy, atomic, chemical, electrical, biological, and large structures, applicable across a range of CT states (5) Utilizing KFE to compress or decompress CT states and form matrices of CT states, facilitating desired dimensional variations (6) Designing matrices based on KFE concepts, considering time as CT state dimensional change, energy as CT state change, and the structural aspects of neutron backbones, proton cores, and electron clouds, enabling control over energy release, redirection, and absorption (7) Incorporating KFE to facilitate the design and manipulation of molecules, including the expansive or contractive features and orientation of atomic or molecular matrices throughout reactions as CT state matrix change occurs (8) Controlling the absorption and spew of CT state exchanges within matrices by strategically shaping reaction chamber parts, adjusting injector jets, and exhaust systems (9) Applying KFE to react chemicals, including fuels, for enhanced matrix changes, maximizing the release of pretime informational change, and optimizing energy utilization (10) Employing KFE to target AuT plasma, stepped transitions, categorization, chemistry, biology, and other relevant features, enabling advancements in various fields (11) Leveraging KFE to achieve balance within matrices, utilizing plasma fulcrums, absorption, and spew mechanisms, and leveraging fractal alignment patterns for efficient CT state interactions (12) Utilizing KFE to design, control, and optimize the proximity of states, the nature of intervening matrices, and the mixing of CT states for desired matrix results.

2. The method of claim 1 further comprising improving fusion processes using Key Fractal Elements (KFE), comprising utilizing KFE to affect change in CT states, interpret matrices of CT states, and design radiation matrices for fusion energy production, wherein the application of KFE enables enhanced fusion reactions, efficient energy release, and improved control over the fusion process.

3. The method of claim 1 further comprising improving fission processes using Key Fractal Elements (KFE), comprising utilizing KFE to affect change in CT states, interpret matrices of CT states, and optimize fission reactions, wherein the application of KFE enables efficient and controlled fission reactions, enhanced energy generation, and improved safety measures.

4. The method of claim 1 further comprising improving chemistry processes using Key Fractal Elements (KFE), comprising utilizing KFE to affect change in CT states, interpret matrices of CT states, and optimize chemical reactions, wherein the application of KFE enables enhanced reaction kinetics, increased reaction selectivity, and improved product yields.

5. The method of claim 1 further comprising using Key Fractal Elements (KFE), comprising utilizing KFE to affect change in CT states, interpret matrices of CT states, and optimize polymerization reactions, wherein the application of KFE enables controlled polymer structure formation, improved polymer properties, and enhanced polymer processing.

6. The method of claim 1 further comprising processes using Key Fractal Elements (KFE), comprising utilizing KFE to affect change in CT states, interpret matrices of CT states, and optimize hydrogen extraction reactions, wherein the application of KFE enables efficient hydrogen release, enhanced hydrogen storage capacity, and improved hydrogen production methods.

7. The method of claim 1 further comprising utilizing KFE to affect change in CT states, interpret matrices of CT states, and design electronic devices and circuits, wherein the application of KFE enables enhanced electronic functionality, improved signal processing, and increased energy efficiency, and optimize battery reactions and materials, wherein the application of KFE enables increased battery capacity, improved charging and discharging rates, and enhanced battery performance.

8. The method of claim 1 further comprising utilizing KFE to affect change in CT states, interpret matrices of CT states, and design quantum computing systems, wherein the application of KFE enables improved quantum information processing, enhanced qubit stability, and increased computational power and optimize AI algorithms and models, wherein the application of KFE enables enhanced AI learning and reasoning capabilities, improved pattern recognition, and advanced decision-making processes.

9. The method of claim 1 further comprising utilizing KFE to affect change in CT states, interpret matrices of CT states, and optimize energy conversion and storage systems, wherein the application of KFE enables increased energy efficiency, improved energy capture and dispersal, and enhanced energy management techniques.

10. The method of claim 1 further comprising a plasma confinement device, a magnetic field generator, and at least one fractal-pattern generating device (e.g., a shaped electrode configured to optimize particle transport and confinement within the plasma) to generate fusion based on KFE.

11. The method of claim 1 further comprising at least one fractal-shaped electrode into the plasma and adjusting the fractal dimensions of the electrode to optimize particle transport and confinement.

12. The method of claim 1 further comprising configuring the plasma confinement device with a fractal-shaped chamber and adjusting the fractal dimensions of the chamber to achieve optimal plasma confinement and stability, resulting in improved fusion reaction rates and energy output.

13. The method of claim 1 further comprising optimizing energy transmission within an electrical power grid of electrical, comprising using KFE in the design of the grid electrical components.

14. The method of claim 1 further comprising the steps of identifying a pretime state of a qubit, applying a pretime operation to the qubit, and measuring the pretime state of the qubit to obtain a pretime result; a pretime computing module configured to identify pretime states of the qubits and apply pretime operations to the qubits, and a pretime measurement module configured to measure pretime states of the qubits to obtain pretime results.

15. The method of claim 1 further comprising a method for evaluating the change in pretime states of a plurality of qubits in a quantum computer, comprising the steps of identifying a pretime state of each qubit, applying a pretime operation to each qubit, measuring the pretime state of each qubit to obtain a pretime result, and analyzing the pretime results to evaluate the change in pretime states of the plurality of qubits to use the pretime results to approximate the change in pretime states of the qubits and wherein the method comprises a quantum computer, a classical computer, and a deep learning neural network used to perform pretime computations, which are then passed to the classical computer for evaluation by the neural network, resulting in faster and more efficient processing.

16. The method of claim 1 further comprising identifying potential drug targets using fractal analysis, comprising obtaining biological data related to a disease or disorder; identifying key fractal elements within the biological data; applying fractal analysis to the identified key fractal elements; and identifying one or more potential drug targets based on the results of the fractal analysis and enhancing the performance of biological systems, comprising: a fractal-based neural network for processing biological data; and a feedback loop for adjusting the fractal-based neural network based on the results of the processing, wherein the feedback loop improves the performance of the biological system by optimizing the fractal-based neural network.

17. A system for controlling compression and decompression of CT states within at least one AuT matrix or between multiple AuT matrices, comprising key fractal elements (KFE) including: (a) stepped AuT fractal transitions governing CT state changes; (b) fractal balance of at least two higher compression CT states about at least one AuT fulcrum comprised of lower compression states; (c) AuT fulcrums defined as lower compression CT states at the overlap of compression of at least two higher compression CT states; (d) absorption and spew of CT states towards compression and decompression within at least one AuT matrix or between multiple AuT matrices; (e) f-series spirals of CT states in and out of alignment for absorption and spew; (f) pairing of higher compression CT states along f-series linear spirals about shared lower compression CT states; (g) folding and unfolding along fractal linear spirals of CT states about at least one AuT fulcrum to achieve compression and decompression; (h) net compression or decompression as a force when observed from the standpoint of time; (i) CT states defined as stepped (golden ratio) fractal dimensional states from common iterated equations; (j) force defined as the result of net winding or unwinding of CT states as viewed from post time CT state perspectives; (k) time defined as stop frame animation resulting from changes in pretime CT states; (1) using AuT as “base logic” of at least one AuT matrix; (m) shifting between higher and lower compression of CT states within the AuT fulcrum; (n) fusion length as a fractal element of CT state transition from compression to decompression; (o) net AuT compression as manifested at different CT states; (p) CT state exchange between at least two AuT matrices in place of collision or field modeling; (q) categorization of AuT matrices based on CT state content, amount of CT states, relative dimensional size, locational area from the perspective of time, AuT plasmas, and fulcrum locations; (r) basing thermodynamic effects of at least one AuT matrix based on categorized CT states within the at least one AuT matrix; (s) treating exchange of lower CT states between at least two higher CT state AuT matrices as the source of interaction; (t) proton positron atomic links for holding electrons; (u) collisions as the exchange of information between at least two AuT matrices; (v) post-collision effects reflecting the net change of CT state and pretime change in each matrix of the at least two matrices; (w) targeting fulcrums and stepped transitions; (x) quantum fractal dimensional change resulting in quantum time; (y) fulcrums as shared CT states between higher CT states; and (z) curvature defined by a solution to fpix for pi with definitive limitations generating the sequential amounts of dimension and curvature in response to net CT state compression.

18. The invention of claim 18 wherein key fractal elements (KFE) are utilized in the creation of new materials and substances by controlling the interaction and fusion of CT states within at least one AuT matrix, thereby enabling the design and production of materials with unique properties and characteristics.

19. The process of claim 19 wherein key fractal elements (KFE) are utilized to model and simulate complex systems and phenomena by controlling the interaction and fusion of CT states within at least one AuT matrix, thereby providing a new method for simulating and predicting the behavior of complex systems.
